(19) United States
(12) Patent Application Publication
(10) Pub. No.: US 2007/0162131 A1
(43) Pub. Date: Jul. 12, 2007

(54) Repair of spinal annular defects

(76) Inventors: Craig D. Friedman, Westport, CT (US); Arindam Datta, Hillsborough, NJ (US); John S. Pedlick, Butler, NJ (US); Yong Song, Sunnyvale, CA (US)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 11/475,444

(22) Filed: Jun. 26, 2006

Related U.S. Application Data
(63) Continuation-in-part of application No. PCT/US04/43455, filed on Dec. 23, 2004.

(51) Int. Cl. A61F 2/44

(52) U.S. Cl. 623/17.11

(57) ABSTRACT

The invention relates to the repair of spinal annular defects. An apparatus comprises a scaffold comprised of a biodurable, resiliently compressible, elastomeric reticulated composition to obliterate spinal/vertabral connective tissue defects, to obliterate spinal-annular nuclear tissue defects, and for spinal annulo-nucleoplasty regeneration. The implant comprises an at least partially cylindrical member.

### CROSS-REFERENCE TO RELATED APPLICATIONS

This application is based upon co-pending, commonly assigned U.S. patent application Ser. No. 10/746,563, filed Dec. 24, 2003, and is a continuation-in-part of PCT patent application Serial No. PCT/US04/43455, filed Dec. 23, 2004, each of which is incorporated herein by reference in its entirety.

### FIELD OF THE INVENTION

This invention relates to the repair of spinal annular defects. More particularly, this invention relates to a method and composition for the repair of spinal annular defects and annulo-nucleoplasty reconstruction.

### BACKGROUND OF THE INVENTION

Back pain is one of the most common and often debilitating conditions affecting millions of people. Some forms of back pain are muscular in nature and may be simply treated by rest, posture adjustments and painkillers. For example, lower back pain (LBP) is a very common condition that may be caused by unusual exertion or injury. Unusual exertion such as heavy lifting or strenuous exercise may result in back pain due to a pulled muscle, a sprained muscle, a sprained ligament, a muscle spasm, or a combination thereof. An injury caused by falling down or a blow to the back may cause bruising. These forms of back pain are typically non-chronic and may be self-treated and cured in a few days or weeks.

Other types of non-chronic back pain may be treated by improvements in physical condition, posture and/or work conditions. Being pregnant or otherwise being significantly overweight may cause LBP. A mattress that does not provide adequate support may cause back pain in the morning. Working in an environment lacking good ergonomic design may also cause back pain. In these instances, the back pain may be cured by eliminating the underlying cause. Whether it is excess body weight, a bad mattress, or a bad office chair, these forms of back pain are readily treated.

It is estimated that over ten million people in the United States alone suffer from persistent back pain. Approximately half of those suffering from persistent back pain are afflicted with chronic disabling pain, which seriously compromises a person's quality of life and is the second most common cause of worker absenteeism. Further, the cost of treating chronic back pain is very high, even though the majority of sufferers do not receive treatment due to health risks, limited treatment options, and/or inadequate therapeutic results. Thus, chronic back pain has a significantly adverse effect on a person's quality of life, on industrial productivity, and on heath care expenditures.

Some forms of back pain are the result of disorders directly related to the spinal column, which disorders are not readily treated. While some pain-causing spinal disorders may be due to facet joint degradation or degradation of individual vertebral masses, disorders associated with the intervertebral discs are predominantly affiliated with chronic back pain (referred to as disc-related pain). The exact origin of disc related pain is often uncertain, and although some episodes of disc related pain may be eased with conservative treatments such as bed-rest and physical therapy, future episodes of disc related pain are likely to occur periodically.

There are a number of suspected causes of disc-related pain, and, in any given patient, one or more of these causes may be present. However, the ability to accurately diagnose a specific cause or locus of pain is currently difficult. Because of this uncertainty, many of the causes of disc-related pain are often lumped together and referred to as degenerative disc disease (DDD).

A commonly suspected source of disc-related pain is physical impingement of the nerve roots emanating from the spinal cord. Such nerve root impingement may have a number of different underlying causes, but nerve root impingement generally results from either a disc protrusion or a narrowing of the intervertebral foramina (which surround the nerve roots).

As a person ages, their intervertebral discs become progressively dehydrated and malnourished. Due to the combination of aging and continued stressing, the discs begin to degenerate. With continued degeneration, or an excessive stressing event, or both, the annulus fibrosus of a disc may tear, forming one or more fissures (also referred to as fractures). Such fissures may progress to larger tears, which allow the gelatinous material of the nucleus pulposus to flow out of the nucleus and into the outer aspects of the annulus. The flow of the nucleus pulposus to the outer aspects of the annulus may cause a localized bulge or herniation.

When herniation of the nucleus/annulus occurs in the posterior portions of the disc, nerve roots may be directly and physically impinged by the bulge. In more extreme or progressed instances of annular tears, the nuclear material may escape, additionally causing chemical irritation of the nerve roots. Dependent upon the cause and nature of the disc protrusion, the condition may be referred to as a disc stenosis, a disc bulge, a herniated disc, a prolapsed disc, a ruptured disc, or, if the protrusion separates from the disc, a sequestered disc.

Dehydration and progressive degeneration of a disc also leads to thinning of the disc. As the thickness of the disc reduces, the intervertebral foraminae become narrow. Because the nerve roots pass through the intervertebral foraminae, such narrowing may mechanically entrap the nerve roots. This entrapment can cause direct mechanical compression or it may tether the roots, causing excessive tension to the roots during body movement.

Nerve root impingement most often occurs in the lumbar region of the spinal column since the lumbar discs bear significant vertical loads relative to discs in other regions of the spine. In addition, disc protrusions in the lumbar region typically occur posteriorly because the annulus fibrosus is radially thinner on the posterior side than on the anterior side and because normal posture places more compression on the posterior side. Posterior protrusions are particularly problematic since the nerve roots are posteriorly positioned relative to the intervertebral discs. Lower back pain due to nerve root irritation not only results in strong pain in the region of the back adjacent the disc, but may also cause sciatica, or pain radiating down one or both legs. Such pain may also be aggravated by such subtle movements as coughing, bending over, or remaining in a sitting position for an extended period of time.

Another suspected source of disc-related back pain is damage and irritation to the small nerve endings which lie in close proximity to or just within the outer aspects of the annulus of the discs. Again, as the disc degenerates and is subjected to stressing events, the annulus fibrosus may be damaged and form fissures. While these fissures can lead to pain via the mechanisms described above, they may also lead to pain emanating from the small nerve endings in or near the annulus, due to mechanical or chemical irritation at the sites of the fissures. The fissures may continue to irritate the small nerve endings, as their presence causes the disc to become structurally weaker, allowing for more localized straining around the fissures. This results in more relative motion of edges of the fissures, increasing mechanical irritation. Because it is believed that these fissures have only limited healing ability once formed, such irritation may only become progressively worse.

A common treatment for a disc herniation is a discectomy, a procedure wherein the protruding portion of the degenerated disc is surgically removed. However, discectomy procedures have an inherent risk since the portion of the disc to be removed is immediately adjacent the nerve root, and any damage to the nerve root is clearly undesirable. Furthermore, discectomy procedures are not always successful long term because scar tissue may form and/or additional disc material may subsequently protrude or reherniate from the disc space as the disc deteriorates further. The recurrence of a disc herniation may necessitate a repeat discectomy procedure, along with its inherent clinical risks and less than perfect long term success rate. Thus, a discectomy procedure, at least as a stand-alone procedure, is clearly not an optimal solution.

Discectomy is also not a viable solution for DDD when no disc/nuclear herniation is involved. As mentioned above, DDD causes the entire disc to degenerate, narrowing the intervertebral space and shifting the load to the facet joints. If the facet joints carry a substantial load, the joints may degrade over time and be a different cause of back pain. Furthermore, the narrowed disc space can result in the intervertebral foramina surrounding the nerve roots directly impinging on one or more nerve roots. Such nerve impingement is very painful and cannot be corrected by a discectomy procedure. Furthermore, a discectomy does not always address pain caused by annular fissures or post-surgical defects, which may cause direct mechanical irritation to the small nerve endings near or just within the outer aspect of the annulus of a damaged disc.

As a result of the limitations of a discetomy, spinal fusion, particularly with the assistance of interbody fusion cages, has become a preferred secondary procedure, and in some instances, a preferred primary procedure. Spinal fusion involves permanently fusing or fixing adjacent vertebrae. Hardware in the form of bars, plates, screws, and cages may be utilized in combination with bone graft material to fuse adjacent vertebrae. Spinal fusion may be performed as a stand-alone procedure, or it may be performed in combination with a discectomy procedure. By placement of the adjacent vertebrae in their normal position and fixing them in place, relative movement there between may be significantly reduced and the disc space may be restored to its normal condition. Thus, theoretically, aggravation caused by relative movement between adjacent vertebrae may be reduced if not eliminated.

The success rate of spinal fusion procedures is certainly less than perfect for a number of different reasons, none of which are well understood. In addition, even if spinal fusion procedures are initially successful, they may cause accelerated degeneration of adjacent discs since the adjacent discs must accommodate a greater degree of motion. The degeneration of adjacent discs simply leads to the same problem at a different anatomical location, which is clearly not an optimal solution. Furthermore, spinal fusion procedures are invasive to the disc, risk nerve damage, and, dependent upon the procedural approach, are technically complicated (endoscopic anterior approach), invasive to the bowel (surgical anterior approach), and/or invasive to the musculature of the back (surgical posterior approach).

Another procedure that has limited clinical success or has been less than clinically totally successful is total disc replacement with a prosthetic disc. This procedure is also very invasive to the disc, and, dependent upon the procedural approach, either invasive to the bowel (surgical anterior approach) or invasive to the musculature of the back (surgical posterior approach). In addition, the procedure may actually complicate matters by creating instability in the spine, and the long-term mechanical reliability of prosthetic discs has yet to be demonstrated.

Many other medical procedures have been proposed to solve the problems associated with degenerative discs or disc protrusions. However, many of the proposed procedures have not been clinically proven, and some of the allegedly beneficial procedures have controversial clinical data. There is a substantial need for improvements in the treatment of spinal disorders, particularly in the treatment of disc related pain associated with a damaged or otherwise unhealthy disc, specifically the repair or reconstruction of disc defects or annulo-nucleoplasty reconstruction. This can potentially can be more beneficial if the medical procedures are conducted preferably during the early stages of treatment of DDD and possibly in conjunction with discetomy.

### OBJECTS OF THE INVENTION

It is an object of the invention to provide a method and apparatus for the repair of spinal annular defects.

It is also an object of the invention to provide a method and apparatus for annulo-nucleoplasty reconstruction.

It is a further object of the invention to provide a method and composition for annulo-nucleoplasty regeneration.

It is a yet further object of the invention to provide a method of repairing spinal annular defects where a polymeric or metallic substantially cylindrical member is inserted into the spinal annulus.

It is a yet further object of the invention where a polymeric or metallic substantially cylindrical member is inserted into the spinal annulus to promote annulo-nucleoplasty reconstruction.

It is a yet further aspect of the invention to provide an implant for spinal annular repair, which comprises:

a base member and

a retention component coupled to the base member and adapted for implantation and fixation into spinal annular tissue,

wherein the retention component is resistive to expulsion from the spinal annular tissue.

These and other objects of the invention will become more apparent from the discussion below.

### SUMMARY OF THE INVENTION

The invention described and claimed below relates to the repair of spinal annular defects. According to the invention, a substantially cylindrical member is inserted through an opening in the spinal annulus to the extent that the distal portion of the substantially cylindrical member extends into the spinal nuclear defect. The substantially cylindrical member is comprised of a biodurable reticulated elastomeric material that expands to seal the opening or obliterate the defect and provides the retention member being resistive or a component being resistive to expulsion from the spinal annular tissue. Optionally the cylindrical member can comprise one or more metal or polymer retention components to assist in maintaining the sealing ability of the substantially cylindrical member to resist its expulsion from the spinal annular tissue. These metal or polymer components may engage the annular tissue, the annular inner wall, the nuclear space, or the nucleo-annular interface, or any combination thereof.

The present invention addresses repairing spinal annular defects by providing improved devices and methods for the treatment of spinal disorders. The improved devices and methods of the present invention specifically address disc-related pain, progressive disc degeneration, and/or reherniation of nuclear material, particularly in the lumbar region, but may have other significant applications not specifically mentioned herein. For purposes of illustration only, and without limitation, the present invention is discussed in detail with reference to the treatment of damaged discs in the lumbar region of the adult human spinal column. Optionally, the device may be used for damaged discs in the thoracic and cervical region of the adult human spinal column or in damaged discs in vertebrate animals.

As will become apparent from the detailed description below, the improved devices and methods of the present invention reduce, if not eliminate, back pain while maintaining near normal anatomical motion. Specifically, the present invention provides an annular repair, reconstruction of the surgically created or existing annular tear, and/or annulo-nucleoplasty regeneration or reconstruction mechanism, while permitting relative movement of the vertebrae adjacent the damaged disc. The present invention provides for reinforcement of the surgically created or existing annular tear. The devices of the present invention are particularly well suited for minimally invasive methods of implantation.

The devices of the present invention provide three distinct functions. First, the reinforcement devices mechanically stabilize and strengthen the annular portion of the spinal disc to minimize, if not eliminate, chronic irritation of local nerve roots and nerve endings adjacent to the periphery of the disc annulus. Second, the devices radially and/or circumferentially conform to the surgically created or enlarged tear and/or pathologic present fissures, fractures, and tears of the disc, thereby preventing the prolapse of extra spinal disc tissue such as nerves and muscle, thereby potentially facilitating healing. And third, the devices may be used to stabilize the nuclear portion of the disc after a discectomy procedure to reduce the need for a subsequent operation or treatment due to rehemiation.

In an exemplary embodiment, the present invention provides disc reinforcement in which a device of the invention is implanted into the annulus of an intervertebral disc. The implantation method may be performed by an open surgical procedure or by a minimally invasive surgical procedure or by the use of sheath, trocar, or cannula, optionally with visualization through an endoscope, or through an endoscopic instrument or endoscope such as an arthroscope, laproscope, or cystoscope. The present invention provides a number or tools to facilitate percutaneous implantation. One or more reinforcement members may be implanted, for example, posteriorly, anteriorly, and/or laterally, and may be oriented circumferentially or radially. As such, the reinforcement members may be used to stabilize the annulus and/or a portion of the annulus so as to reduce recurrent bulges and/or obliterate annular tracts.

The implant device may be sized to pass through a sheath, trocar, cannula, or endoscope and/or may have a tubular cross-section to facilitate advancement over a stylet. The implant device preferably includes a body portion sized to fit in an opening in the annulus and a retention component for engaging the annulus or the nuclear space or the nucleo-annular interface and limiting relative movement there between. Both the body portion and the retention component can provide resistive force to prevent expulsion from the spinal annular tissue. The retention component, sometimes referred to as an anchor, may be disposed at the distal portions of the implant body, or may extend over the entire length of the body. The anchor or retention component to engage the annulus tissue may comprise a portion of the cylinder or can be shaped as an expanded cylinder or as a spherical, mushroom-shaped, etc., shape or the anchor or retention component may comprise fixation elements or members such as threads, wings, clips, loops, barbs, etc., which may have a variable pitch or angle to facilitate compression of the anchor or retention component annulus during implantation. The biodurable reticulated elastomeric material that comprises the implant device will allow for tissue ingrowth and proliferation and bio-integrate the implant device to the annular defect. The tissue ingrowth and proliferation is expected to provide resistive force to prevent expulsion from the spinal annular tissue. The biodurable reticulated elastomeric material that comprises the implant device allows for tissue ingrowth from the annulus and from the surrounding tissue and will seal the annular defect and in one embodiment provide a permanent sealing of the aperture. The implant device may incorporate chemical and/or biological agents. The implant device may comprise a biocompatible metal such as stainless steel or a super elastic (nickel titanium) alloy. Alternatively, the implant device may comprise a polymer or a reinforced polymeric structure. As a further alternative, the implant device may comprise a bioabsorbable material.

According to one embodiment of the invention, an apparatus comprises a scaffold comprised of a biodurable, resiliently compressible, elastomeric reticulated composition to repair and/or regenerate spinal/vertebral connective tissue defects.

According to another embodiment of the invention, an apparatus comprises a scaffold comprised of a biodurable, resiliently compressible, elastomeric reticulated composition to repair and/or reconstruct and/or regenerate spinal-annular nuclear tissue defects.

According to another embodiment of the invention, an apparatus comprises a tissue scaffold comprised of a biodurable, resiliently compressible, elastomeric reticulated composition for spinal annulo-nucleoplasty repair.

According to another embodiment of the invention, an apparatus comprises an at least partially cylindrical member.

According to another embodiment of the invention, the elastomeric composition is partially hydrophobic.

According to another embodiment of the invention, the elastomeric composition comprises polyurethane.

According to another embodiment of the invention, the elastomeric composition comprises a polycarbonate polyurethane or a polycarbonate polyurethane-urea.

According to another embodiment of the invention, the elastomeric composition comprises a reticulated elastomeric matrix comprising a plurality of pores, the pores having an average diameter or other largest transverse dimension of at least about 20 μm.

According to another embodiment of the invention, the pores have an average diameter or other largest transverse dimension of from about 20 μm to about 150 μm.

According to another embodiment of the invention, the pores have an average diameter or other largest transverse dimension of from about 150 μm to about 250 μm.

According to another embodiment of the invention, the pores have an average diameter or other largest transverse dimension of from about 250 μm to about 500 μm.

According to another embodiment of the invention, the reticulated elastomeric matrix is configured to permit cellular ingrowth and proliferation into the elastomeric matrix.

According to another embodiment of the invention, the reticulated elastomeric matrix is endoporously coated with a coating material selected to encourage cellular ingrowth and proliferation.

According to another embodiment of the invention, the coating material comprises a foamed coating of a biodegradable material, the biodegradable material comprising collagen, fibronectin, elastin, hyaluronic acid or mixtures thereof.

According to another embodiment of the invention, the implantable device comprises a plurality of elastomeric matrices.

According to another embodiment of the invention, the apparatus comprises a structural or retention component adapted to maintain the scaffold in a desired location.

According to another embodiment of the invention, the structural or retention component comprises a compressible element at least partially within the scaffold that compresses during delivery and expands or releases upon delivery to engage tissue.

According to another embodiment of the invention, the structural or retention component comprises a longitudinal shaft member with fixation elements comprising umbrella-like spokes.

According to another embodiment of the invention, the structural or retention component comprises one or more arrangements of fixation elements comprising radial projections.

According to another embodiment of the invention, the apparatus can be rotated in one direction to engage tissue and in another direction to disengage tissue.

According to another embodiment of the invention, a system for treating a spinal annular defect comprises an implantable apparatus and a delivery means.

According to another embodiment of the invention, the delivery means is a cannula, trocar, catheter, or endoscope.

According to another embodiment of the invention, a method of treating spinal annular defects comprises:

(a) inserting an implantable apparatus into the lumen of a delivery means;

(b) advancing the distal tip of the delivery means into an opening in an annulus;

(c) advancing the apparatus through the lumen into the opening; and

(d) withdrawing the delivery means, whereby the apparatus expands into the opening.

According to another embodiment of the invention, the delivery means is a trocar, cannula, or catheter, with visual assistance through an endoscopic instrument.

According to another embodiment of the invention, an implant for spinal annular repair comprises:

a base member and

a retention member integral with or coupled to the base member and adapted for implantation and fixation into spinal annular tissue,

wherein the retention member is resistive to expulsion from the spinal annular tissue.

According to another embodiment of the invention, the retention member has an implantation configuration and a fixation configuration.

According to another embodiment of the invention, the implantation configuration and the fixation configuration of the retention commember comprise respective configurations that are substantially similar.

According to another embodiment of the invention, the retention component comprises at least one fixation element for resistance to expulsion.

According to another embodiment of the invention, the at least one fixation element comprises at least two fixation elements.

According to another embodiment of the invention, the base member comprises a distal portion and at least one fixation element is disposed at least in part at the distal portion of the base member.

According to another embodiment of the invention, the at least one fixation element is disposed substantially along a major dimension of the implant.

According to another embodiment of the invention, the resistance to expulsion in the fixation configuration is greater than the resistance to implantation in the spinal annular tissue when in the implantation configuration.

According to another embodiment of the invention, the retention component is at least partially contained in the base member.

According to another embodiment of the invention, the at least one fixation element comprises at least one material selected from at least one of the group consisting of a biocompatible metal, a polymer, a reinforced polymer, a reticulated material and a bioabsorbable material.

According to another embodiment of the invention, the spinal annular tissue comprises a defect, the implant is deployed into the defect, and when the implant is in the defect, the base member of the implant substantially seals or obliterates the defect.

According to another embodiment of the invention, the base member comprises a material selected from the group consisting of a biodurable material, a biodurable reticulated resilient elastomeric material, a resiliently compressible material, an elastomeric material, a reticulated material, and a material comprising two or more of the properties of the foregoing materials.

According to another embodiment of the invention, the base member comprises a reticulated resilient elastomeric material.

According to another embodiment of the invention, the elastomeric material comprises a biodurable material.

According to another embodiment of the invention, the elastomeric material is selected from the group consisting of polycarbonate polyurethane urea, polycarbonate polyurea urethane, polycarbonate polyurethane, polycarbonate polysiloxane polyurethanes, polycarbonatepolysiloxane polyurethane ureas, polysiloxane polyurethanes, polysiloxane polyurethaneureas, polycarbonate hydrocarbon polyurethanes, polycarbonate hydrocarbon polyurethane ureas, and mixtures of two or more thereof.

According to another embodiment of the invention, the reticulation comprises a plurality of pores having a largest transverse dimension of from about 20 pm to about 500 pm.

According to another embodiment of the invention, the elastomeric material has an elongation to break of at least about 46%.

According to another embodiment of the invention, the elastomeric material has an elongation to break of at least about 125%.

According to another embodiment of the invention, the elastomeric material has an elongation to break of at least about 194%.

According to another embodiment of the invention, the elastomeric material has an elongation to break of at least about 215%.

According to another embodiment of the invention, the elastomeric material has an ultimate tensile elongation of at least about 25%.

According to another embodiment of the invention, at least a portion of the retention component has a resilient compressibility that allows the implantable device to be compressed from a first relaxed configuration to a second configuration during implantation and to expand to a third working configuration when in the fixation position.

According to another embodiment of the invention, at least one portion of the retention component recovers from the second configuration to a size selected from the group consisting of at least 50%, at least 60%, and at least 90%, of the size of the relaxed configuration.

According to another embodiment of the invention, the retention component is at least partially contained in a base member.

According to another embodiment of the invention, the base member comprises a biodurable reticulated resilient elastomeric material.

According to another embodiment of the invention, an implant is adapted for use in repairing a spinal annulo-nuclear defect.

According to another embodiment of the invention, the annulo-nuclear defect comprises an interface between the nucleus and the defect, and the retention component has at least a portion for seating at the interface between the nucleus and the defect.

According to another embodiment of the invention, an implant comprises a retention component to resist an expulsion force.

According to another embodiment of the invention, an implant comprises at least one fixation element.

According to another embodiment of the invention, the base member comprises interconnected networks of voids and/or pores for encouraging cellular ingrowth.

According to another embodiment of the invention, the retention component and the base member are integral to the implant.

According to another embodiment of the invention, the reticulated elastomeric material of the body is treated with a substance that encourages tissue ingrowth.

According to another embodiment of the invention, the implant is adapted to mechanically stabilize and strengthen the annular portion of the spinal annular tissue and reduce chronic irritation of local nerve roots and nerve endings adjacent to the periphery of the disc annulus.

According to another embodiment of the invention, the implant radially and/or circumferentially conforms to a surgical and/or pathologic present fissure, fracture or tear of the spinal annular tissue, thereby facilitating healing.

According to another embodiment of the invention, the implant stabilizes the nuclear portion of the spinal annular tissue after discectomy and reduces the need for subsequent surgery or treatment due to reherniation.

According to another embodiment of the invention, the retention component has a bias structure, in which a first energy is stored when in an implantation configuration, and has a resistance to expulsion when in a fixation configuration; and the retention component has a second stored energy component when in a fixation configuration.

According to another embodiment of the invention, the retention component comprises a proximal end and a distal end, and the retention component comprises at least one projection located in the vicinity of the distal end.

According to another embodiment of the invention, the at least one projection has a respective major axis having a directional component that is oriented towards the proximal end.

According to another embodiment of the invention, the retention component comprises one or more fixation elements that project into the spinal annular tissue when the retention component is in a fixation configuration.

According to another embodiment of the invention, the fixation elements are at least partially compressed when the retention component is in the implantation configuration and the compression is at least partially released when the retention component is in the fixation configuration.

According to another embodiment of the invention, the fixation elements are at least partially collapsed when in the retention component is in the implantation configuration and at least partially expanded when the retention component is in the fixation configuration.

According to another embodiment of the invention, the one or more fixation elements do not project beyond the surface of the body when in the implantation configuration.

According to another embodiment of the invention, the retention component comprises a longitudinal member.

According to another embodiment of the invention, a method for securing a medical apparatus, the apparatus comprising a retention component adapted for deployment into a spinal annular tissue defect, the retention component having a implanation configuration and a fixation configuration, and being resistive to expulsion in the fixation configuration, comprises:

(a) positioning the apparatus with respect to the spinal annular tissue defect with a delivery device;

(b) deploying the apparatus; and

(c) at least partially fixating the retention component.

According to another embodiment of the invention, a method for treating a spinal annular defect with an apparatus comprising a body having a proximal cylindrical portion and a distal portion, comprises:

(a) inserting the apparatus into the lumen of a delivery device;

(b) advancing the distal tip of the delivery device into the defect in an annulus;

(c) advancing the apparatus from the delivery device to the defect; and

(d) fixating the apparatus in the defect.

According to another embodiment of the invention, an apparatus for securing a medical apparatus directed to spinal annular repair, comprises a retention component coupled to the apparatus and adapted for positioning in a spinal annular tissue, the retention component comprising a main portion and a radial component for retaining the apparatus.

According to another embodiment of the invention, the retention component is formed integral to the implant.

According to another embodiment of the invention, the retention component comprises two or more at least partially radially extending projections.

According to another embodiment of the invention, the retention component comprises a cylindrical shape.

According to another embodiment of the invention, the retention component comprises a portion of a substantially frusto conical surface.

According to another embodiment of the invention, the retention component comprises a longitudinal member and a radially extending projection coupled to the longitudinal member.

According to another embodiment of the invention, the retention component comprises a substantially cylindrical portion.

According to another embodiment of the invention, the retention component comprises a coil portion.

According to another embodiment of the invention, an apparatus for spinal annular repair comprises a member comprising resilient elastomeric material adapted for retaining the implant in an annular defect, the annular defect having an annular defect wall, wherein the member has an implantation configuration and a fixation configuration, wherein the member is adapted to be in a first state prior to being placed in the implantation configuration, and in a second state when in the fixation configuration, and wherein the member forms a seal with the annular wall when in the second state.

According to another embodiment of the invention, the first state comprises a state of compression in at least one dimension, and the second state comprises a state of at least partial reexpansion.

According to another embodiment of the invention, the seal comprises a frictional seal.

According to another embodiment of the invention, the member of resilient elastomeric material comprises a reticulated material.

According to another embodiment of the invention, the member of resilient elastomeric material comprises a biodurable material.

According to another embodiment of the invention, the annular defect further comprises an annular nuclear opening, wherein the member protrudes through the annular defect beyond the annular nuclear opening when in the fixation position, wherein the implant further comprises a portion coupled to the member adapted to protrude beyond the annular nuclear opening when the member is in the fixation configuration, and wherein the portion protruding beyond the annular nuclear opening is expanded further than the member when in the second reexpanded position.

According to another embodiment of the invention, the portion protruding beyond the annular nuclear opening has a cross-sectional shape that differs from the cross-sectional shape of the base member.

According to another embodiment of the invention, a retention component is coupled to the base member and is adapted for implantation and fixation into a spinal annular tissue.

According to another embodiment of the invention, the fixation is into the annular defect wall.

According to another embodiment of the invention, a retention component is coupled to the base member and is adapted for implantation and fixation into a spinal annular tissue.

According to another embodiment of the invention, the fixation is into an annular tissue at the annular nuclear opening.

According to another embodiment of the invention, an implant, for use in treating a defect in spinal annular tissue, comprises a material having a composition and structure adapted for application to the defect and for biointegration into the spinal annular tissue when applied to the defect.

According to another embodiment of the invention, the structure comprises a scaffold.

According to another embodiment of the invention, the scaffold comprises a reticulated structure.

According to another embodiment of the invention, the reticulated structure is resiliently compressible.

According to another embodiment of the invention, the resiliently compressible reticulated structure comprises an elastomeric material.

According to another embodiment of the invention, the elastomeric material comprises a biodurable material.

According to another embodiment of the invention, application to the defect comprises insertion into the defect.

According to another embodiment of the invention, an implant further comprises a retention component for securing the implant with respect to the defect.

According to another embodiment of the invention, the implant is secured with respect to the defect to facilitate biointegration of the implant with respect to the defect.

According to another embodiment of the invention, the implant, when inserted into the defect, is dimensioned with respect to the defect to at least partially resist expulsion from the defect.

According to another embodiment of the invention, the retention component comprises a radial component.

According to another embodiment of the invention, the radial component comprises a portion of increased diameter when inserted in the defect.

According to another embodiment of the invention, the radial component comprises at least one radially projecting element.

According to another embodiment of the invention, an implant comprises a fixation element for fixing the implant with respect to the defect.

According to another embodiment of the invention, an implant comprises a fixation element for fixing the implant with respect to the defect into which it is inserted.

According to another embodiment of the invention, the structure of the implant comprises interconnected networks of voids and/or pores encouraging cellular ingrowth of spinal annular tissue.

According to another embodiment of the invention, biointegration of the implant with the spinal annular tissue due to cellular ingrowth presents resistance to migration of the implant.

According to another embodiment of the invention, an implant for spinal annular repair comprises:

a retention component to be integral with or coupled to a base member and adapted for implantation and fixation into a spinal annular tissue,

wherein the retention component has an implantation configuration and a fixation configuration, the retention component being resistive to expulsion from the spinal annular tissue when in the fixation configuration.

### BRIEF DESCRIPTION OF THE DRAWINGS

FIG. 1 illustrates a superior (top) cross-sectional view of a healthy disc;

FIGS. 2 and 3 each illustrate a superior (top) cross-sectional view of a degenerated disc;

FIG. 4 is a partially cross-sectional view of an embodiment of an at least partially cylindrical implant according to the invention;

FIG. 5 is a partially cross-sectional view of another embodiment of an at least partially cylindrical implant according to the invention;

FIG. 6 is a partially cross-sectional view of a further embodiment of an at least partially cylindrical implant according to the invention;

FIG. 7 is a cross-sectional view across the line 7-7 of the embodiment of the invention shown in FIG. 6;

FIG. 8 is a partially cross-sectional view of another embodiment of the invention in position in an opening in an annulus;

FIG. 9 is a partially cross-sectional view of a variation of the embodiment shown in FIG. 8;

FIG. 10 is a lateral view of an embodiment of an implant of the invention having radial projections;

FIG. 11 is a cross-sectional view along the line 11-11 in FIG. 10;

FIGS. 12 to 14 represent cross-sectional views of delivery of the embodiment of the invention set forth in FIGS. 10 and 11;

FIG. 15 is a lateral view of a cylindrical base member useful in an implant according to the invention, and FIG. 16 is an oblique view of that same member;

FIG. 17 is a lateral view of another cylindrical base member useful in an implant according to the invention, and FIG. 18 is an oblique view of that same member;

FIGS. 19 to 22 are each a view of a fixation element useful in an implant according to the invention;

FIGS. 23 and 24 A and 24 B are views of an embodiment of an implant of the invention;

FIGS. 25 to 30 are partially cross-section views of implants according to the invention and their delivery;

FIGS. 31A to 31N represent views of fixation elements useful according to the invention;

FIGS. 32 to 34 are views of the structure and delivery of an implant according to the invention;

FIG. 35 is a partially cross-sectional view of another embodiment of the invention;

FIGS. 36 to 45 represent partially cross-sectional views of additional embodiments of the invention and their delivery;

FIG. 46 is a partially cross-section view of another embodiment of the invention;

FIGS. 47 to 56 represent partially cross-section views of additional embodiments of the invention and their delivery;

FIG. 57 is a partially cross-sectional view of another embodiment of the invention;

FIGS. 58 to 60 represent partially cross-sectional views of another embodiment of the invention and its delivery;

FIGS. 61 and 62 are each a partially cross-sectional view of an embodiment of the invention;

FIGS. 63 and 64 are each views of a fixational element useful in an implant according to the invention;

FIG. 65 is a partially cross-sectional view of an implant useful according to the invention;

FIGS. 66 and 67 are each a micrograph of material prepared according to Example 1;

FIGS. 68 and 69 are each a micrograph of material prepared according to Example 2;

FIG. 70 is a micrograph of an embodiment of the invention four weeks after placement;

FIG. 71 is a detailed view of a section of the micrograph in FIG. 19;

FIG. 72 is the histology of the annulus in mini-swine spine in which device was implanted and the animal sacrificed after 6 weeks;

FIG. 73 is the histology of the annulus in mini-swine spine in which device was implanted and the animal sacrificed after 6 weeks;

FIG. 74 shows unextruded device flush with external annulus wall harvested from a sheep spine 4 weeks after implantation of device in sheep spine; and

FIG. 75 is the histology of the annulus harvested from a sheep spine 4 weeks after implantation of device in sheep spine.

### DETAILED DESCRIPTION OF THE INVENTION

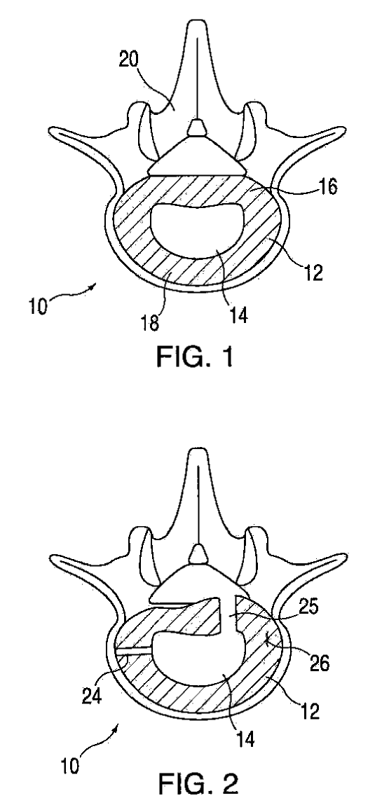

The invention can perhaps be better appreciated from the drawings. FIG. 1 is a simplified representation of a cross-section of a spinal disc 10 that comprises an annulus fibrosis or annulus 12 surrounding a nucleus pulposus or nucleus 14. The posterior annulus 16 is generally thinner than the anterior annulus 18, which may account for the higher incidence of posterior disc protrusions.

A common theory is that each intervertebral disc 10 forms one support point and the facet joints of the spinal column (not shown) form two support points of what may be characterized as a three-point support structure between adjacent vertebrae 20. However, in the lumbar region, the facet joints are substantially vertical, leaving the disc 10 to carry the vast majority of the load. As between the annulus 12 and the nucleus 14 of the disc 10, it is commonly believed that the nucleus 14 bears the majority of the load. This belief is based on the theory that the disc 10 behaves much like a balloon or tire and the nucleus 14 bears somewhat of the majority of the load wherein the annulus 12 merely serves to contain the pressurized nucleus 14 and supports a somewhat smaller proportion of the total load. The annulus 12 comprises 60% of the total disc 10 cross-sectional area and is made of 40-60% organized collagen in the form of a laminated structure. By contrast, the nucleus 14 only comprises 40% of the total disc 10 cross-section and is made of 18-30% collagen in the form of a relatively homogenous gel. In reality, both the nucleus 14 and annulus 12 play important and critical roles in the load-bearing mechanism of the disc 10.

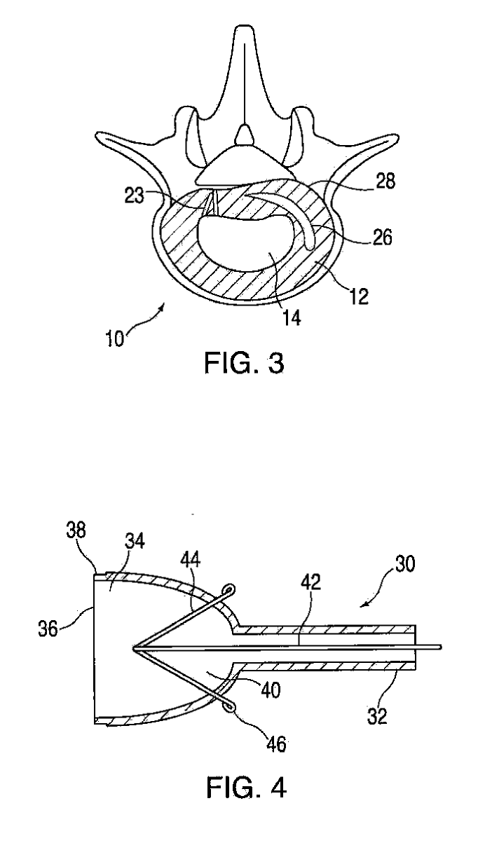

Intervertebral disc 10 becomes progressively dehydrated and malnourished with age, as shown in FIGS. 2 and 3. In combination with continued stressing from load-bearing and/or resisting outward pressure from nucleus 14, disc 10 begins to degenerate. With continued degeneration, or an excessive stressing event, annulus 12 of disc 10 may tear, forming one or more radial fissures 23 or tracts 24 or circumferential fissures 26, which may progress to larger tears. Larger tears may allow the gelatinous material of nucleus 14 to flow out of nucleus 14 through a fissure 24 and into the outer aspects of annulus 12. Nuclear material that escapes through an advanced tear may cause further mechanical irritation and additionally cause chemical irritation of a nerve root.

The flow of nucleus 14 to the outer aspects of annulus 12 may cause a localized bulge 28. A posterior bulge 28 may result in direct impingement of a nerve root (not shown).

A nerve root may also be compressed or tethered by a narrowing of the intervertebral foraminae, resulting from a loss in disc height caused by sustained degeneration of disc 10. Small nerve endings (not shown) in or near the perimeter of annulus 12 may also be mechanically or chemically irritated at the sites of fissures 24, 26. In all cases, degeneration of the disc eventually leads to disc-related pain of some origin.

Lumbar discetomy is one of the most common spine procedures. There are many methods available for the surgeon to accomplish removal of disc material. In most of these procedures a pathway through the annular fibrosus to the nucleus pulposus is either present pathologically as an annular tear 24 or an aperture or tear 25 is created via an annulotomy during the surgical procedure. Clinically since this annular defect or annular aperature never heals properly or does not heal completely by itself, possibly due to avascular environment, causing potential disc rehemiation, repairing an annular tear or the defect in the anulotomy has been suggested as a potentially valuable method to improve discectomy outcomes. Optimal healing or physical repair of the annular fibrosus with approximation and reinforcement of the anulotomy could be beneficial in improving overall patient outcome and ultimately reducing the need for repeat surgery at the same disc level. This can be achieved by sealing annular defect, repairing the annular defect, reconstructing the annular defect or obliterating the annular defect or a combination thereof by placing or positioning an implant in the defect. As a result, sealing and/or obliteration of the annular defect or aperture leads to reinforcing and stabilization of the annulus tissue. At least part of the implant is placed or positioned at or within the defect, and in a preferred mode, the part of the implant that is placed or positioned at or within the defect is placed in a conformal fashion with the contour of the defect. In another preferred embodiment, the part of the implant that is placed or positioned at or within the defect is in conformity with the various surfaces of the defect in contact with the implant. The implantation site of the device can potentially be the site of herneation or in close proximity to the site of herneation in case of treatment of herniated disc or otherwise damaged or attenuated parts of the disc. In one case, the site of herneation and associated discetomy can be the posterior-lateral side of the intervertebral disc.

In an embodiment of the invention shown in FIG. 4, a partially cylindrical device 30 comprises a cylindrical portion 32 and an attached expanded, at least partially spherical portion 34. Portion 34 may be entirely spherical or it may optionally have a substantially flat surface 36 bordered by edge 38. Optionally, the attached expanded portion 34 may be entirely cylindrical. In one embodiment, the attached expanded portion 34 may be any other suitable shape that has at least one transverse dimension or diameter larger than the diameter of the cylindrical portion 32. Optionally, portion 34 can act as a retention member, the retention member being resistive to expulsion from the spinal annular tissue. Portions 32 and 34 are both solid, although optionally each may contain a longitudinal lumen (not shown) to facilitate threading member 30 over a wire or stylet (not shown). Portions 32 and 34 are both solid in that they have a distinct shape and boundary but can be made from porous, filled, homogeneous or non-homogeneous materials. In a preferred mode, portions 32 and 34 can be made from biodurable reticulated elastomeric resilient matrix. Optionally, portion 34 can act as a retention member or component, the retention member or component being resistive to expulsion from the spinal annular tissue. Optionally, portion 32, being preferably larger in diameter or having a transverse section larger than those of the annular defect, can act as a retention member or component, the retention member or component being resistive to expulsion from the spinal annular tissue. Also, device 30 may optionally contain a retention or fixation member 40, comprising a longitudinal member or shaft 42 and collapsible/expandable spokes/radial members in the fixation elements, 44. Optionally, fixation elements 44 can deform slightly during delivery or not deform at all during delivery. Preferably the proximal end of each fixation elements 44 has a tissue fixation member 46 that contacts the inner portion of the annulus when fixation element 44 expand, to hold or fix device 30 in position within the annular defect that is being repaired or reconstructed.

Retention or fixation member 40 has one, two, three, or four, preferably two or three, members 44 and a central shaft 42 as shown in FIG. 4. Optionally retention or fixation member 40 can be shaped as an umbrella. The fixation element 44 can be partially collapsed or deform within a sheath, trocar, cannula, or endoscope during delivery and contact the inner portion of the annulus when they expand after delivery to hold or fix elastomeric reticulated device 30 or device 48 in position. Alternatively, fixation element 44 deform slightly or do not deform at all during delivery and in one embodiment are able to push and/or outwardly stretch the annular tissue surrounding the annulotomy hole without damage as retention or fixation member 40 is placed in the spinal annular tissue. In one embodiment, fixation element 44 can substantially recover to their original shape after delivery and placement. In another embodiment, members 44 retain shape and/or size substantially similar to their original shape and/or size after delivery and placement. When pushed and/or outwardly stretched during delivery, the annular tissue surrounding the annulotomy hole recovers to its original shape and integrity after delivery of device 30.

Retention or fixation member 40 can have a range of dimensions depending on specific applications. The range of dimensions of the different parts are as follows: the angle between central shaft 42 and spokes or fixation element 44 comprises from about 15° to about 60°, when the spokes are fully opened. The length of each spoke 44 ranges from about 3 mm to about 10 mm, preferably from about 4 mm to about 7 mm. The cross-section of spokes 44 can be cylindrical, elliptical, square, rectangular, or any other polygonal shape. The diameter of spokes' 44 cross-section or one side of the spoke 44 cross-section ranges from about 0.5 mm to about 5 mm. The end-to-end distance of the spokes 44 when the spokes 44 are fully opened or spread out or is at its maximum distance ranges from about 4 mm to about 15 mm. The cross-section of central shaft 42 can be cylindrical, elliptical, square, rectangular, or any other polygonal shape with the diameter of the central shaft 42 cross-section or one side of the of the central shaft cross-section ranging from about 0.5 mm to about 5 mm. The overall length of central shaft 42 of the umbrella shaped retention or fixation member (including the head and the stem) can range from about 4 mm to about 15 mm.

Spokes or fixation element 44 can be regularly spaced from each other or they could be “paired” as cross-pieces. For example, adjacent spokes 44 could be separated by 60° and 120° to form an “X” pattern. In another example, adjacent spokes 44 could be separated by 30° or 45°. Also, in another embodiment, shaft 42 could extend in the direction from spokes 44 opposite to the direction shown in FIG. 4. In yet another embodiment spokes 44 may be arcuate, pointing in the proximal direction, rather than straight as shown in FIG. 4.

Retention or fixation member 40 is comprised of a physiologically acceptable metal such as nitinol or stainless steel and, after compression, expands to form an umbrella-like shape. In another embodiment, retention or fixation member 40 preferably is comprised of a degradable or non-degradable polymer and, after compression, expands to form an umbrella-like shape. In another embodiment, retention or fixation member 40 preferably is comprised of a degradable or non-degradable polymer and does not change its shape during delivery. In another embodiment, retention or fixation member 40 has substantially similar size and shape prior to delivery, during delivery, and after placement in the annular defect.

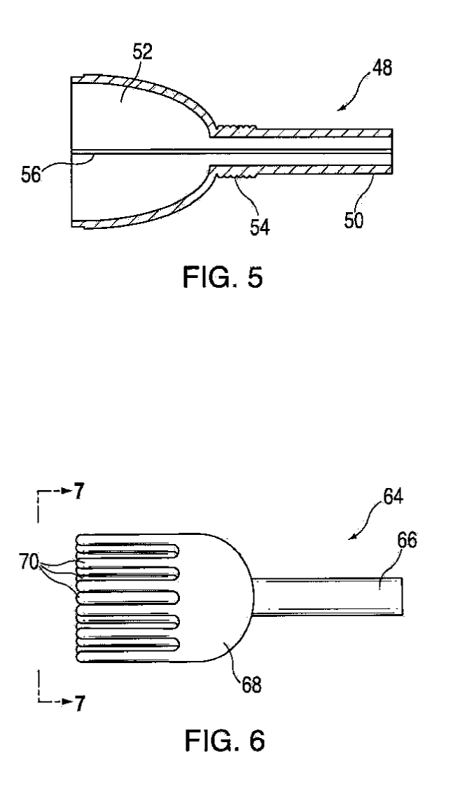

In the embodiment of the invention shown in FIG. 5, a partially cylindrical device 48 comprises a cylindrical portion 50 and a goblet- or mushroom-shaped distal portion 52. In one embodiment, the mushroom-shaped distal portion 52. can also be cylindrical in shape. In another embodiment, the mushroom-shaped distal portion 52 can also be partially spherical in shape or any other suitable shape that has at least one transverse dimension larger than the diameter of the cylindrical portion 50. In general, the diameter or the largest transverse dimensions of the distal portion 52 is greater than the diameter of the cylindrical portion 50. Optionally, portion 52 can act as a retention member or component, the retention member or component being resistive to expulsion from the spinal annular tissue. Optionally, cylindrical portion 50 has ridges or projections 54 that aid in fixedly positioning device 48 in an annular fissure, especially at the inner portion of the fissure. Optionally device 48 has a lumen 56 to facilitate positioning device 48 over a stylet or wire (not shown).

The dimensions of the shaped and sized devices made from the elastomeric matrix can vary depending on the application. In one embodiment, major dimensions of a device, such as device 30 or device 48, prior to being compressed and delivered, are from about 5 mm to about 30 mm in one direction and from about 5 mm to about 30 mm in another direction. In another embodiment, major dimensions of a device, such as device 30 or device 48, prior to being compressed and delivered are from about 8 mm to about 25 mm in one direction and from about 8 mm to about 25 mm in another direction. The length of a cylindrical portion of a device, such as device 30 or device 48, according to the invention is expected to be from about 6 mm to about 14 mm, since that is approximately the typical radial thickness of a patient's annulus. The diameter or the largest transverse dimension of the cylindrical portion of a device, such as cylindrical part 32 or cylindrical part 50, according to the invention is expected to be from about 5 mm to about 30 mm, preferably from about 8 mm to about 20 mm. The diameter or the largest transverse dimension of the partial cylindrical or partial spherical portion of a device, such as expanded portion 34 or mushroom-shape distal portion 52, according to the invention is expected to be from about 8 mm to about 40 mm, preferably from about 10 mm to about 30 mm. The elastomeric matrix can exhibit compression set upon being compressed and transported through a delivery-device, e.g., a trocar, cannula, or catheter, with assisted visualization. In another embodiment, compression set and its standard deviation are taken into consideration when designing the pre-compression dimensions of the device.

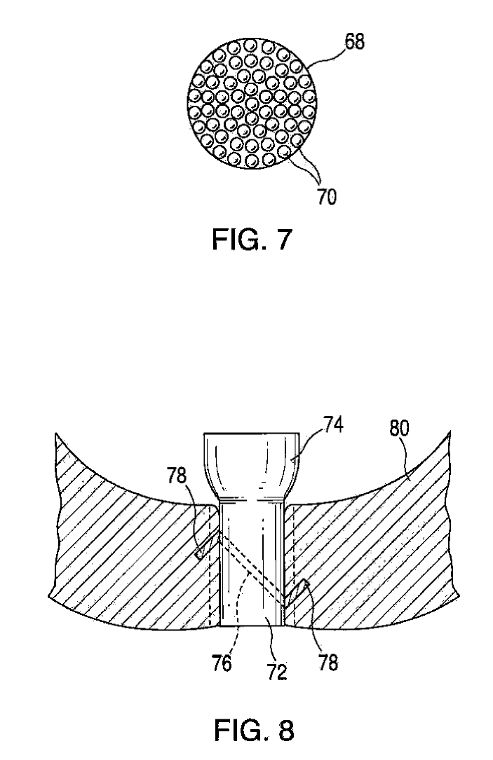

The embodiment of the invention shown in FIGS. 6 and 7 is an at least partially cylindrical member 64 that comprises a cylindrical member 66 and a distal semi-spherical portion 68 that comprises distally extending projections 70. Preferably projections 70 comprise spaghetti-like shapes suitable for cell propagation.

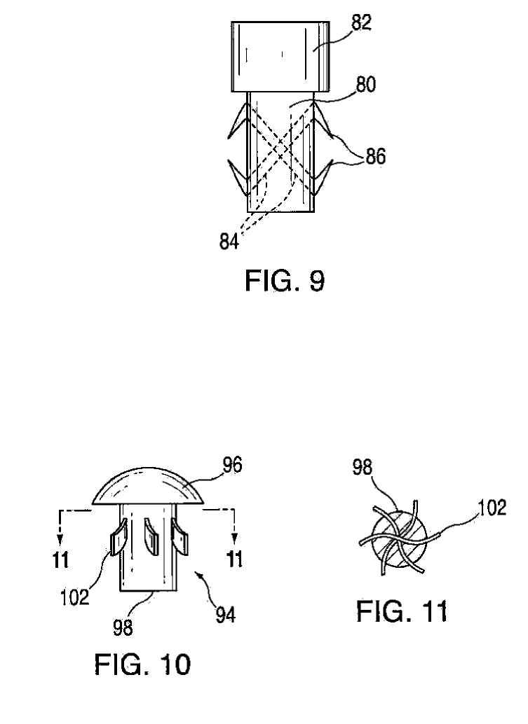

FIG. 8 represents an embodiment of the invention where base member 74 has one or more crossmembers 76 that have projections 78, intended to engage annular tissue 80. Crossmember 76 can have integral projections 78, so that the crossmember 76 and a projection 78 are inserted, preferably at an angle, into base member 74 prior to delivery, where preferably projection 78 collapses slightly to permit insertion, preferably through a sheath, trocar, cannula, or endoscope. Alternatively, projections 78 are attached by glue, “fit”, or other suitable fixtation after crossmember 76 is positioned within base member 74. As is shown in the uncompressed base member 82 depicted in FIG. 9, there could be two or more sets of crossmembers 84 and projections 86.

In FIG. 10 an implant 94 is shown in uncompressed condition with a mushroom-shaped distal tip portion 96 and a cylindrical portion 98. Cylindrical portion 98 has radially-extending projections or prongs 102. As shown in the cross-sectional view of FIG. 11, implant 94 has six projections 102. However, there could be from 2 to as many as 16 or more projections 102, preferably from about 4 to 12. Optionally there could be projections 102 on more than one plane of cylindrical portion 98, preferably 2 or 3 planes altogether, such as proximal neck and/or mid-shaft and/or distal shaft.

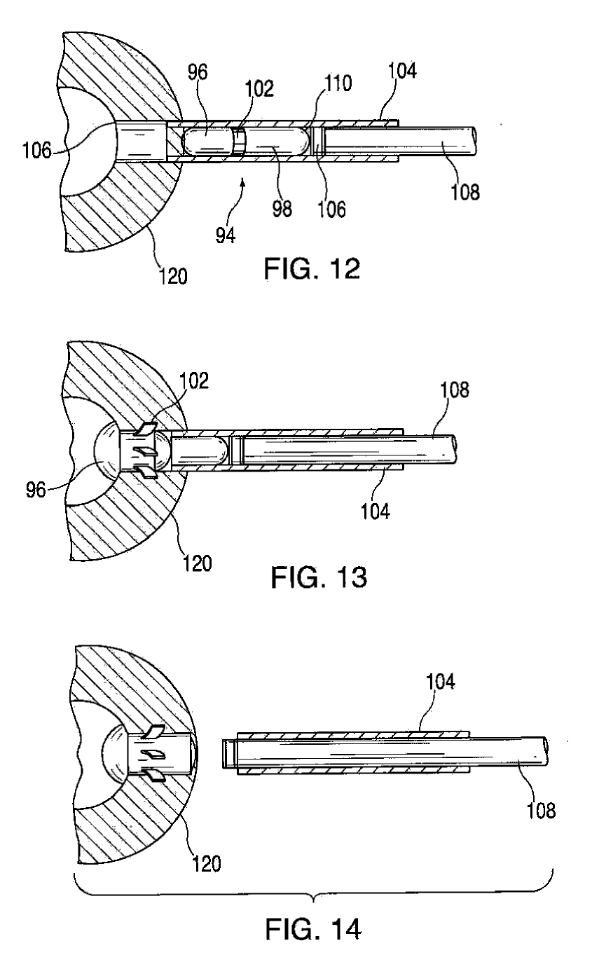

Delivery of implant 94 is shown in FIGS. 12 to 14. Implant 94 in a compressed state is preloaded into a rigid or substantially rigid tubular member 104. Projections 102 fold around cylindrical portion 98, and the distal portion 106 of a pushing rod or member 108 is positioned adjacent to the proximal surface 110 of cylindrical portion 98. The distal tip 114 of tubular member 104 is positioned in or adjacent to an opening 116 in annulus 120.

As shown in FIGS. 13 and 14, pushing member 108 pushes retention or fixation member 94 distally so that retention or fixation member 94 fills and engages opening 116. Retention or fixation member distal portion 96 expends into the cavity 122 of annulus 120 and seals opening 116. Projections 102 are designed so that tubular member 104 can be rotated or twisted to cause projections 102 to expand into or engage the tissue of annulus 120 to secure retention or fixation member 94 in position. When tubular member 104 is withdrawn from opening 116, the bottom portion 124 of cylindrical member 98 fills out the remainder of opening 116. It is within the scope of the invention that when retention or fixation member 92 is twisted or rotated in the opposite direction, projections 102 will disengage so that retention or fixation member 92 could be revived or repositioned. It is also within the scope of the invention that an retention or fixation member can be held, maintained, or retained in position by other retaining means, such as sutures, staples, clips, or the like.

In one aspect of the invention, an implant or device to be implanted or positioned in an annular fissure or surgically created annular tear can comprise an implant system having (1) a substantially cylindrical base member and (2) a separate retention member that is positioned within or on the outer surface of the base member, the retention member being resistive or component being resistive to expulsion from the spinal annular tissue. In one embodiment, base member can be shapes other than cylindrical or partially cylindrical. The retention member can be integral to or separate from the base member. Optionally the retention member can reside in the nuclear space 14, at the surgically created annular defect, at the surgically created annular tear or at the nucleo-annular interface or at the site of the fissures 24 or 25 located in annulus 12 after delivery or placement. In a preferred mode, the retention member will substantially reside in the nuclear space 14. Optionally the retention member can reside in the nuclear space 14 or at the site of the fissures 24 or 25 located in annulus 12 the after delivery or placement. In a preferred mode, the retention member will reside in the nuclear space 14. To facilitate delivery through a delivery member such as a sheath, trocar, cannula, or endoscope, the base member or the base device is compressed to fit within the sheath, trocor, cannula, or endoscope to be delivered but upon ejection from the sheath, trocar, cannula, or endoscope, resumes its unstressed shape or shape substantially similar to shape prior to compression. To facilitate delivery through a delivery member such as a sheath, trocar, cannula, or endoscope, the retention member must be capable of being compressed, rotated, flattened, or otherwise configured to fit within the sheath, trocar, cannula, or endoscope to be delivered but to then resume its unstressed shape upon ejection from the sheath, trocar, cannula, or endoscope. In another embodiment, the delivery through a delivery member such as a sheath, trocar, cannula, or endoscope, the retention member deforms slightly or does not deform at all during delivery. In some cases when the retention member deforms slightly or does not deform at all during delivery, and it is able to push and/or outwardly stretch the annular tissue surrounding the annulotomy hole without damage as it is placed in the spinal annular tissue. In one embodiment, the retention member substantially recovers to its original shape after delivery and placement. In another embodiment, the retention member retains a shape and/or size substantially similar to their original shape and/or size after delivery and placement. In another embodiment, the retention member does not recover substantially to its original size and/or shape but still provides resistive force to prevent expulsion from the spinal annular tissue. When pushed and/or outwardly stretched during delivery of the implant, the annular tissue surrounding the annulotomy defect, made surgically or naturally, recovers substantially to it original shape and integrity after delivery of retention member and the base member.

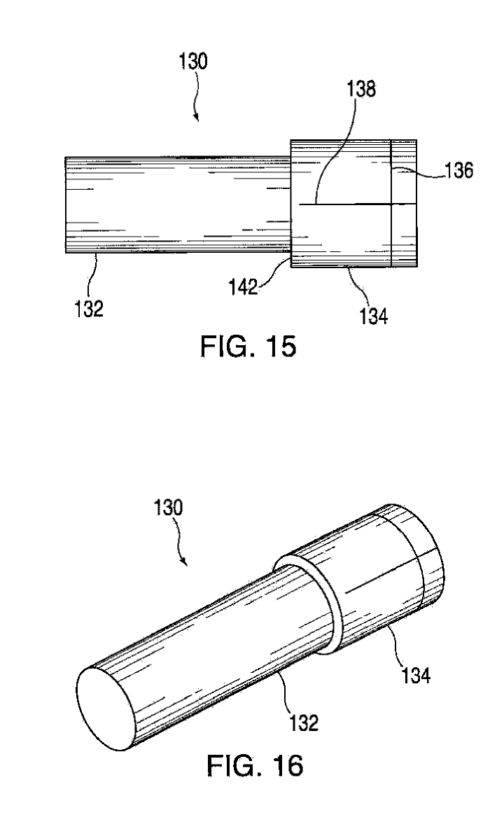

Two embodiments of a base member for an implant according to the invention are shown in FIGS. 15 to 18. In FIG. 15, a base member 130 comprises a proximal cylindrical section 132 and a distal mushroom section 134. Optionally, a proximal cylindrical section 132 and a distal mushroom section 134 may have non-circular cross-sections such as squares, rectangles, triangle, etc. or irregular cross sections. Mushroom section 134 may optionally have a circular slit cut 136 or a slit cut 138. The outer diameter of cylindrical section 132 could be from about 3 to about 12 mm, and its length could be from about 6 to about 20 mm. The outer diameter of mushroom section 134 could be from about 5 to about 15 mm, and its length could be from about 4 to about 15 mm. Optionally the mushroom section 134 can act as a retention member and the retention member being resistive to expulsion from the spinal annular tissue. In one embodiment, the diameter of the proximal cylindrical section 132 and diameter of the distal mushroom section 134 section can be same or similar. In this case, when placed in the annular fissure with a diameter less than the diameter of base member, the part of the base member that is not constrained by the annular fissure, the will effectively serve as a mushroom, i.e., act as a retention member or component, the retention member or component being resistive to expulsion from the spinal annular tissue. Optionally, portion 132, being preferably larger in diameter or having a transverse section larger than the diameter or the largest transverse section of the annular defect, can act as a retention member or component, the retention member or component being resistive to expulsion from the spinal annular tissue. Circular slit cut 136 from about 6 to about 15 mm. Circular slit cut 136 can be positioned from about 5 to about 10 mm from the proximal surface 142 of mushroom section 134, and the depth of circular slit cut 136 will be from about 0.25 to about 2.0 mm. Slit cut 138 will end from about 0.25 to about 3 mm from surface 142.

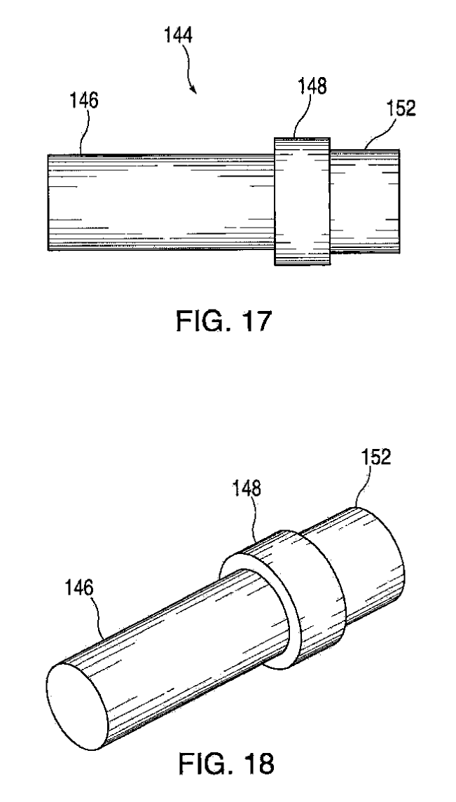

In FIG. 17, a base member 144 comprises a proximal cylindrical section 146, an expanded middle cylindrical or mushroom section 148, and a distal cylindrical or top hat section 152. The outer diameter of proximal cylindrical section 146 is from about 3.0 to about 12.0 mm, and its length is from about 6.0 to about 20.0 mm. The outer diameter of middle cylindrical section 148 is from about 5.0 to about 15.0 mm, and its length is from about 4.0 to about 15 mm. The outer diameter of distal cylindrical section is from about 3.0 to about 12.0 mm, and its length is from about 3.0 to about 5.0 mm. Optionally the diameter of middle cylindrical section 148 can act as a retention member and the retention member being resistive to expulsion from the spinal annular tissue. Optionally, the diameter of proximal cylinder 146 being preferably larger in diameter or having a transverse section larger than those of the annular defect, can act as a retention member or component, the retention member or component being resistive to expulsion from the spinal annular tissue. In one embodiment, the diameter of the proximal cylindrical section 146 and diameter of middle cylindrical section 148 section can be same or similar. Optionally, a proximal section 146 and a middle section 148 may have non-circular cross-sections such as squares, rectangles, triangle, etc. or irregular cross sections. In one embodiment, the retention members are in at least partial conformity with the defect or aperture or fissure after they are placed or positioned at or within the defect or aperture or fissure. The conformity of the implanted device with the defect or aperture is due to the resilient and elastomeric nature of the retention member.

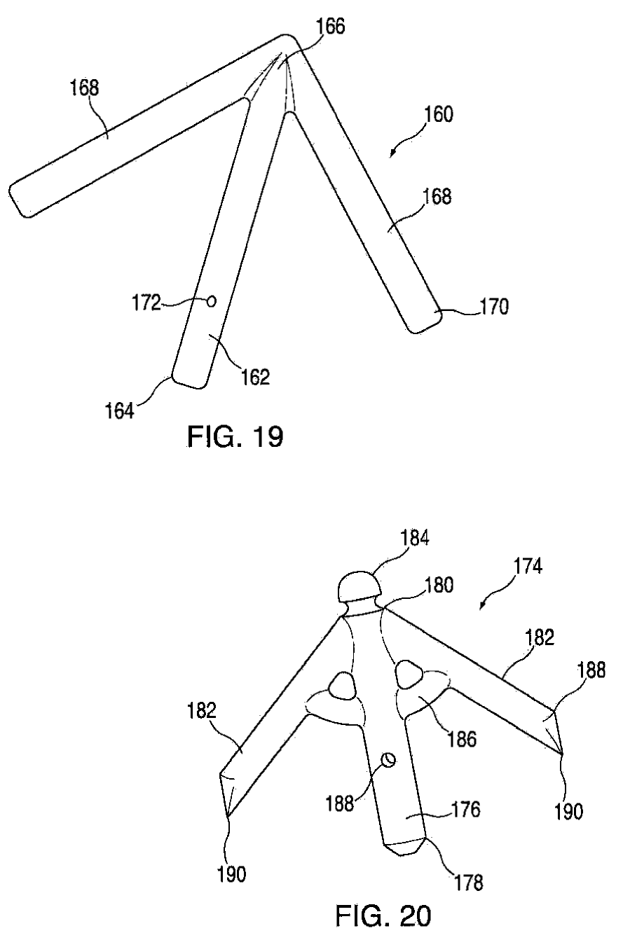

In an aspect of the invention shown in FIG. 19, a fixation member 160 comprises a longitudinal base member 162 having a proximal end 164 and a distal end 166. At distal end 164 there are two or more angularly extending arms 168. Implant or fixation member 160 can acts as a retention member or component being resistive to expulsion from the spinal annular tissue and in one embodiment will be separate from base member. Longitudinal base member 162 optionally has at least one opening 172 to engage a suture string (not shown). The distal tips 170 of arms 168 are shown here to be cylindrical. In one embodiment, the exemplary cross-section of longitudinal base member 162 and arms 168 can be square, rectangle, circular, ellipse, triangle, etc. Cross-section of base member 162 and arms 168 and gusset 184 need not necessarily be uniform. Fixation member 160 can have a range of dimensions depending on specific applications. The range of dimensions of the different parts are as follows: the angle between base member 162 and extending arms 168 is from about 15° to about 75°. The length of each extending arms 168 ranges from about 2 mm to about 10 mm, preferably from about 4 mm to about 7 mm. The cross-section of extending arms 168 can be cylindrical, elliptical, square, rectangular, or any other polygonal shape and its diameter range from about 0.05 mm to about 5 mm. The cross-section of base member 162 can be cylindrical, elliptical, square, rectangular, or any other polygonal shape and its diameter range from about 0.05 mm to about 5 mm. The overall length of base member 162 can range from about 4 mm to about 15 mm.

A variation of fixation member 160 is shown in FIG. 20, where an implant 174 comprises a longitudinal base member 176 having a proximal end 178 and a distal end 180. Implant or fixation member or 174 can acts as a retention member or component being resistive to expulsion from the spinal annular tissue and in one embodiment will be separate from base member. Two or more arms 182 extend angularly from distal end 180, and a protrusion 184 protrudes from distal end 180. Each arm 182 is supported or connected by a gusset 186 that extends from longitudinal base member 174 to each arm 182. Each arm 182 has a proximal end 188 that tapers to a proximally extending point 190. Longitudinal base member 176 optimally has at least one opening 180 to engage a suture string (not shown). In one embodiment, the exemplary cross-section of longitudinal base member 176 and arms 182 and gusset 186 can be square, rectangle, circular, ellipse, triangle, etc. Cross-section of base member 176 and arms 182 and gusset 184 need not necessarily be uniform. Fixation member 174 can have a range of dimensions depending on specific applications. The range of dimensions of the different parts are as follows: the angle between base member 176 and extending arms 182 is from about 15° to about 60°. The length of each extending arms 182 ranges from about 2 mm to about 10 mm, preferably from about 4 mm to about 7 mm. The cross-section of extending arms 182 can be cylindrical, elliptical, square, rectangular, or any other polygonal shape and its diameter range from about 0.05 mm to about 5 mm. The cross-section of base member 176 can be cylindrical, elliptical, square, rectangular, or any other polygonal shape and its diameter range from about 0.05 mm to about 5 mm. The overall length of base member 176 can range from about 4 mm to about 15 mm.

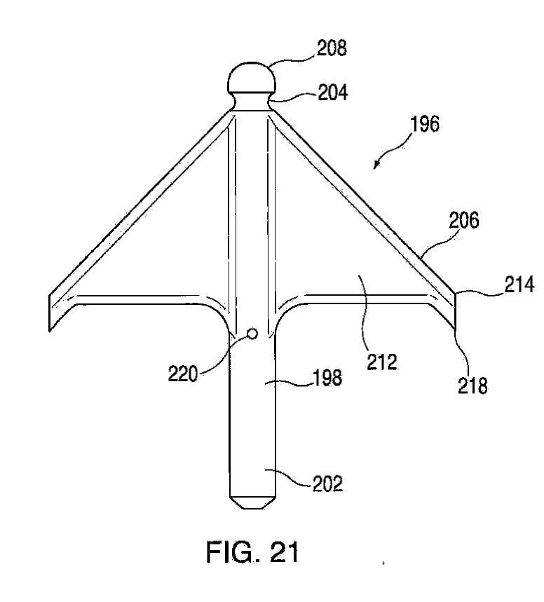

FIG. 21 represents an aspect of the invention where a fixation element 196 comprises a longitudinal member 198 with a proximal end 202 and a distal end 204. Two or more arms 206 extend angularly from distal end 204, and a protrusion 208 extends distally from distal end 204. Implant or fixation member 196 can acts as a retention member or component being resistive to expulsion from the spinal annular tissue and in one embodiment will be separate from base member. A solid support member 212 is positioned between each arm 206 and longitudinal member 198. Each arm 206 has a proximal end 214 that tapers to a proximally extending point 218. Longitudinal member 198 optionally has an opening 220 to engage a suture string (not shown). Fixation member 196 can have a range of dimensions depending on specific applications. The range of dimensions of the different parts are as follows: the angle between longitudinal member 198 and extending arms 206 is from about 15° to about 60°. The length of each extending arms 206 ranges from about 3 mm to about 10 mm, preferably from about 4 mm to about 7 mm. The cross-section of extending arms 206 can be partially be cylindrical, elliptical, square, rectangular, or any other polygonal shape and its diameter range from about 0.05 mm to about 5 mm. The cross-section of longitudinal member 198 can partially be cylindrical, elliptical, square, rectangular, or any other polygonal shape and its diameter range from about 0.05 mm to about 5 mm. The overall length of base member 198 can range from about 4 mm to about 15 mm. Solid support member 212 can optionally have a rectangular or square cross-section.

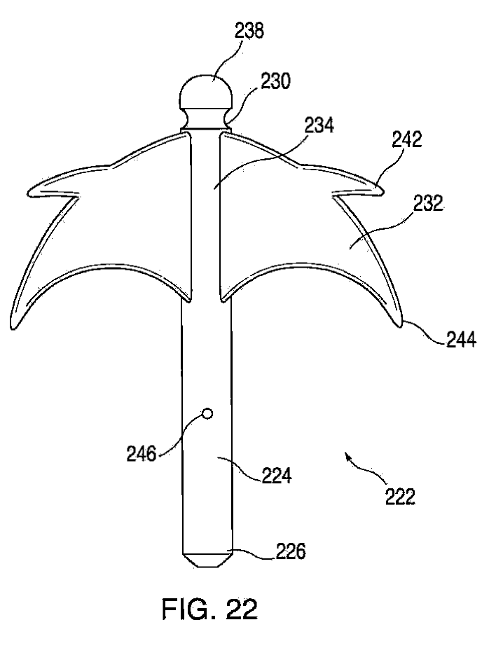

In FIG. 22, a fixation element 222 comprises a longitudinal member 224 with a proximal end 226 and a distal end 230. Implant or fixation member 222 can acts as a retention member or component being resistive to expulsion from the spinal annular tissue and in one embodiment will be separate from base member. Two or more arcuate arm members 232 extend radially from the distal portion 234 of longitudinal member 224, and a protrusion 238 extends distally from distal end 230. Each arcuate arm 232 has optionally one or more barbs 242, and each arcuate arm 232 tapers to a substantially proximally extending point 244. Longitudinal member 224 optionally has at least one opening 246 to engage a suture string (not shown).

In FIGS. 19 to 21, the arms and support members or gussets 184, 212 are shown as straight elements. It is within the scope of the invention that one or more of these elements can optionally be curved, such as concave or convex. Also, fixation elements 160, 174, 196, and 222 are each shown essentially in a planar configuration. It is within the scope of the invention that there can be one or more arms, preferably from 2 to 4, where the arms would be regularly spaced apart. In each of these cases shown on FIGS. 19 to 22, the distances between the distal tips 170 of arms 168, between the proximally extending point 190 of arm 182, proximally extending point 218 of arm 206 and proximally extending point 244 of arm 232 are larger than the maximum dimension, i.e., the diameter or the largest transverse section of the annular defect or annular aperture or annular fissure. When implanted or positioned in an annular defect or fissure, each of the distal tips or extending points 170, 190, 218, and 244 acts optionally as the retention component for implants such as 160, 174, 196, and 222, respectively. The diameter or the largest transverse dimension of the surgically created annular defect, surgically created annular tear, or annular fissure such as 24 or 25 can range from about 1 mm to about 12 mm, preferably from about 3 mm to about 8 mm. The distances between the distal tips 170 of arms 168, between the proximally extending point 190, proximally extending point 218 and proximally extending point 244, can range from about 2 mm to about 14 mm. In another embodiment, the diameter or the largest transverse dimension of the annular fissure or annular defect or annular aperture such as 24 or 25 can range from about 2 mm to about 12 mm but preferably from about 4 mm to about 8 mm. The distances between the distal tips 170 of arms 168, between the proximally extending point 190, proximally extending point 218 and proximally extending point 244 can range from about 3 mm to about 14 mm. The retention members resist expulsion from the spinal annular tissue and preferably reside in the nuclear space 14 after delivery or placement. Optionally, distal tips 170 of arms 168, proximally extending point 190, proximally extending point 218 can be partially engaged or partially embedded in the in the annulus when they extend from the and/or from within the nuclear space 14.

The retention members resisting expulsion can reside in the nuclear space 14, at the surgically created annular defect, at the surgically created annular tear or at the nucleo-annular interface or at the site of the fissures located in annulus 12 the after delivery or placement. In another embodiment, the retention member will substantially reside in the nuclear space 14. In another embodiment, the retention member will at least partially reside in the nuclear space 14. Optionally, distal tips 170, proximally extending point 190, proximally extending point 218 and proximally extending point 244 can be partially engaged or partially embedded in the annulus after placement in the defect. In another embodiment, distal tips 170, proximally extending point 190, proximally extending point 218 and proximally extending point 244 span or are larger in distance than the diameter or the largest transverse dimension of the annular defect, annular tear or annular aperture or annular fissure after placement in the defect. The annular defect, annular tear, annular aperture or annular fissure can be surgically created or present pathologically. Optionally, distal tips 170, proximally extending point 190, proximally extending point 218 and proximally extending point 244 can extend from the and/or within the nuclear space 14. In one embodiment, the retention member substantially recovers to its original shape after delivery and placement. In another embodiment, the retention member retains a shape and/or size substantially similar to their original shape and/or size after delivery and placement. In another embodiment, the retention member does not recover substantially to its original size and/or shape but still provides resistive force to prevent expulsion from the spinal annular tissue.

In all these cases the retention member can be integral or separate from the base member for the implant. In another embodiment, at least part of the base member of the implant is placed or positioned at or within the defect and in a preferred mode, this part of the implant that is placed or positioned at or within the defect is placed in a conformal fashion with the contour of the defect or aperture or fissure. In another preferred embodiment, the part of the base member of the implant that is placed or positioned at or within the defect or aperture or fissure is in conformity with the various surfaces of the defect in contact with this part of the implant. The conformity of the implanted device with the defect or aperture can be ascribed to or happens owing to the resilient and elastomeric nature of the base member.

FIG. 23 shows an exemplary attachment of external or separate fixation or retention member to a substantially cylindrical base member. The attachment can be made with braided fiber, suture, glue, and sealant. The external or separate fixation member such as 174, 196 or 222 can be directly attached to the base member 144 by braided fiber, suture, glue, and sealant or placed in slits in the mushroom head and then attached by braided fiber, suture, glue, and sealant. FIG. 24A and FIG. 24B shows two exemplary positioning of the device containing base member and attached external or separate fixation or retention member placed in an annular defect. In fig. both FIGS. 24A and 24B, the retention member, consists of two parts, one being the external or separate fixation or retention member and the other part being integral to the base member and together they provide forces or functionality necessary for resisting expulsion. In FIG. 24A, the retention member partially reside in the nuclear space and is partially embedded in the annulus after delivery or placement. In FIG. 24B, the retention member partially reside in the nuclear space and is partially engaged in the annulus or just touching the annulus without being embedded into it after delivery or placement.

In one embodiment, the implant can be positioned within annular tract. In another embodiment, the implant can be placed subannularly, i.e., in the nuclear space, as well as within the annular tract. In yet another embodiment, the proximal part of the implant or base member of the implant can be trimmed after delivery and implantation so it is positioned completely within the annular tract. Although not bound by any theory, it is believed that the trimming of the proximal part allows for outward expansion of the device without impinging the nerves or nerve roots.

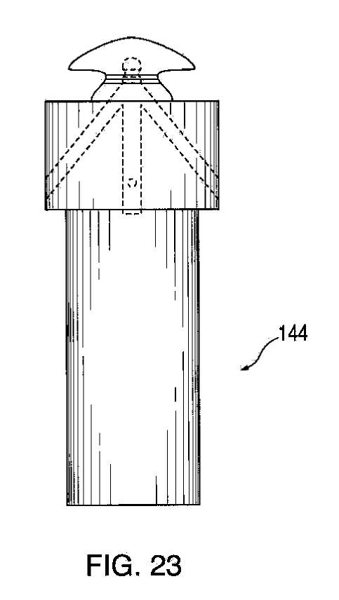

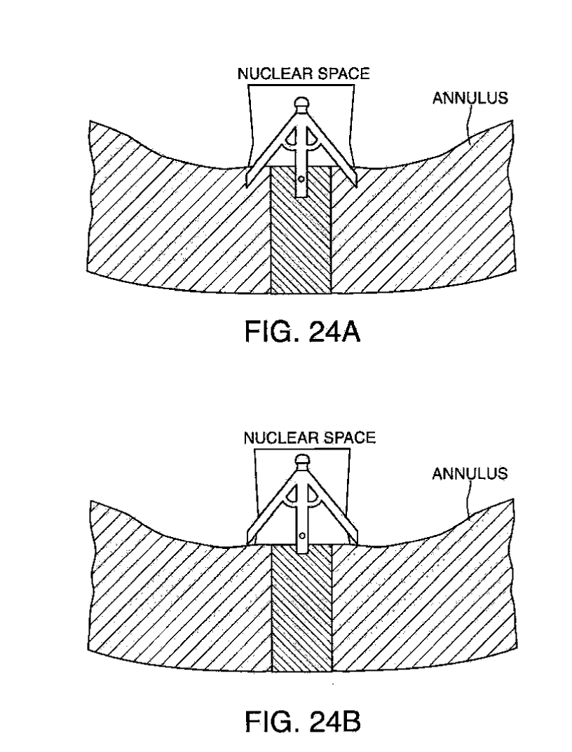

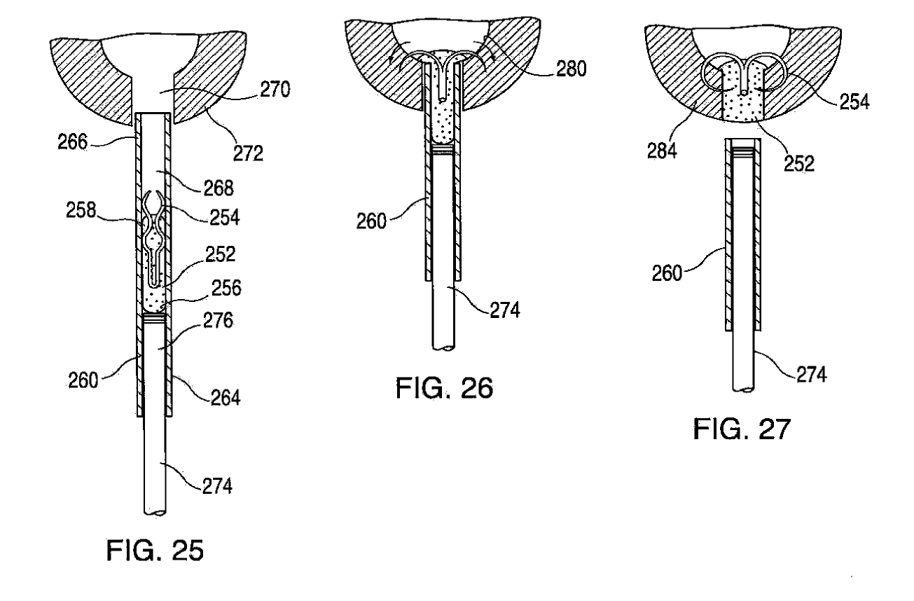

In the embodiment of the invention shown in FIGS. 25 to 27, an implant 252 has two or more loop members 254, a proximal end 256, and a distal end 258. In FIG. 25 implant 252 is shown in a delivery position in a trocar 260 that has a proximal end 264, a distal end 266, and a lumen 268, where loop members 254 are stretched. Trocar distal end 266 is inserted into an opening 270 in an annulus 272. A pusher member 274 having a distal end 276 is inserted into trocar proximal end 264 so that pusher member distal end 276 is adjacent to implant proximal end 256.

When implant 252 is within lumen 268, loop members 254 are constrained by the wall of lumen 268. However, when trocar 260 is advanced distally into annulus opening 270 and then pusher member 274 is advanced distally, as shown in FIG. 26, loop members 254 are released and, due to rotational memory force, engage annulus 242 through annulus inner surface 250, and even penetrate through annulus 272 to implant 252.

As shown in FIG. 27, implant 252 is inserted sufficiently far that loop members 254 engage annulus 272 and implant 252, and implant distal end 258 expands slightly on annulus inner surface 280. Proximal end 256 of implant 252 will be substantially planar with the outer surface 284 of annulus 272.

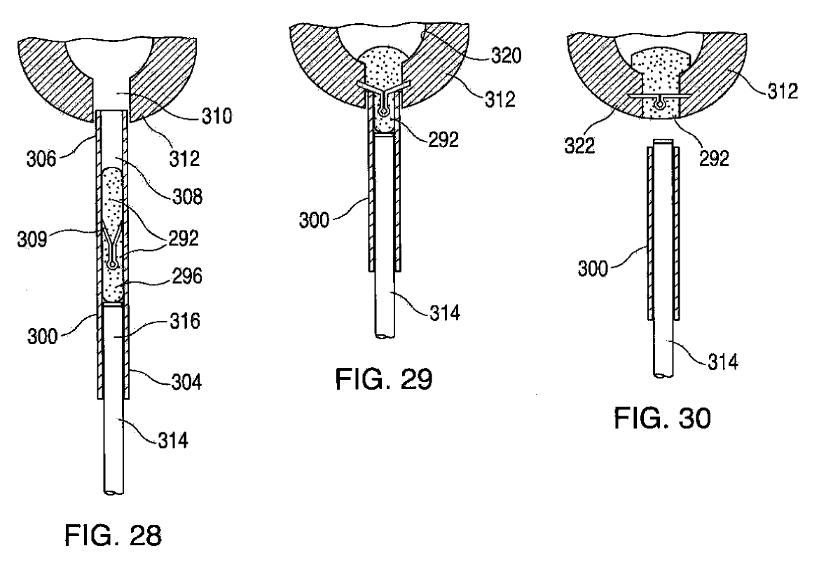

In the embodiment of the invention shown in FIGS. 28 to 30, an implant 292 has two or more staple members 294, a proximal end 296, and a distal end 298. In FIG. 28 implant 292 is shown in a delivery position in a trocar 300 that has a proximal end 304, a distal end 306, and a lumen 308. Trocar distal end 306 is inserted into an opening 310 in an annulus 312. A pusher member 314 having a distal end 316 is inserted into trocar proximal end 304 so that pusher member distal end 316 is adjacent to implant proximal end 296.

When implant 292 is within lumen 308, staple members 294 are constrained by the wall of lumen 308. However, when trocar 300 is advanced distally into annulus opening 310 and then pusher member 314 is advanced distally, as shown in FIG. 29, staple members 294 are released and engage annulus 312.

As shown in FIG. 30, implant 292 is inserted sufficiently far that staple members 294 engage annulus 312 and implant distal end 298 expands slightly on annulus inner surface 320. Proximal end 296 of implant 262 will be substantially planar with the outer surface 324 of annulus 312.

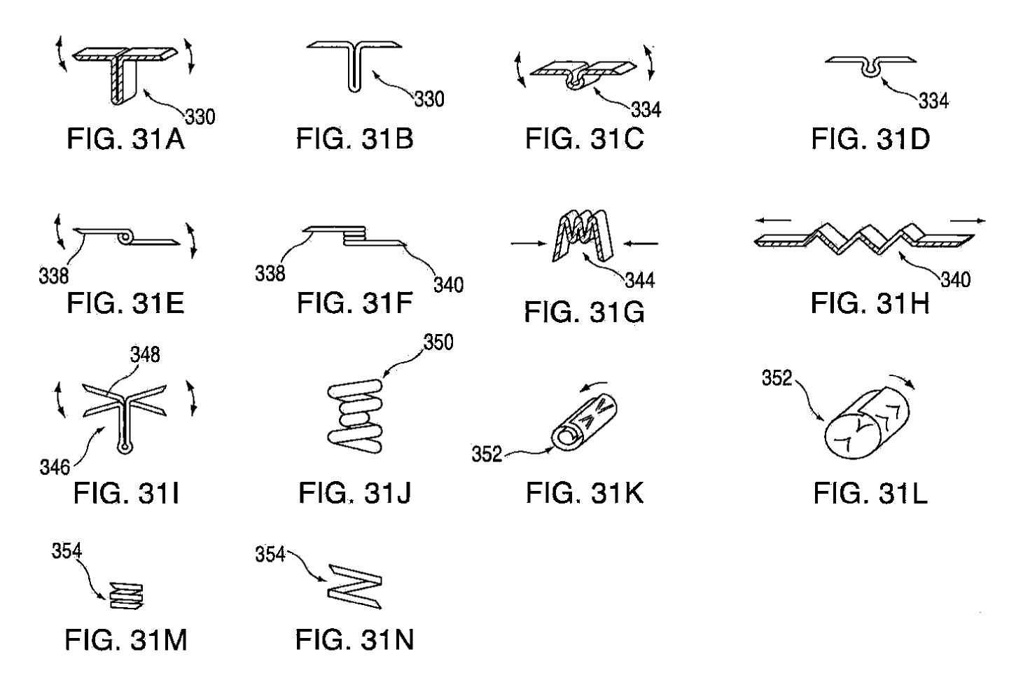

The implants 252 and 292 in FIGS. 25 to 30 each have a fixation or structural element that is released to engage the annulus, similar in function to the fixation elements of FIGS. 19 to 22. It is within the scope of the invention that the implant may comprise other structural elements that expand or reconfigure upon release from the trocar to engage the annulus. Representative examples of such structural elements are set forth in FIGS. 31A to 31 N. In FIG. 31A, a t-shaped structural element 330 would form a y-shaped element in a trocar and then resume its t-shape upon release to engage an annulus. FIG. 31B is a lateral view of t-shaped structural element 330. The wing-shaped structural element 334 of FIG. 31C would form a v-shaped element in a trocar and then resume its wing-shape upon release. FIG. 31D is a lateral view of wing-shaped element 334.

The spring coil structural element 338 shown in lateral and top views in FIGS. 31E and 31F would assume a v-shaped element when compressed in a trocar and assume its normal shape upon release, wherein pointed tips 340 would engage an annulus. The w-shaped structural element 344 shown in FIGS. 31G and 31H would assume the compressed shape of FIG. 31G when compressed in a trocar and then resume the w-shape of FIG. 31H to engage an annulus upon release.

The structural element 346 of FIG. 311 has upper members 348 that are positioned at different levels. Structural element 346 will have an essentially y-shaped configuration upon placement in a trocar. Upon release from the trocar, pointed tips 348 will engage an annulus.

The structural element of FIG. 31J is a spiral coil 350 that can be wound tightly to fit within a trocar. There, upon release from the trocar, coil 350 will resume its unwound shape with a larger diameter, to engage an annulus. The structural elements 352 and 354 shown in FIGS. 31K to 31N work in similar fashion.

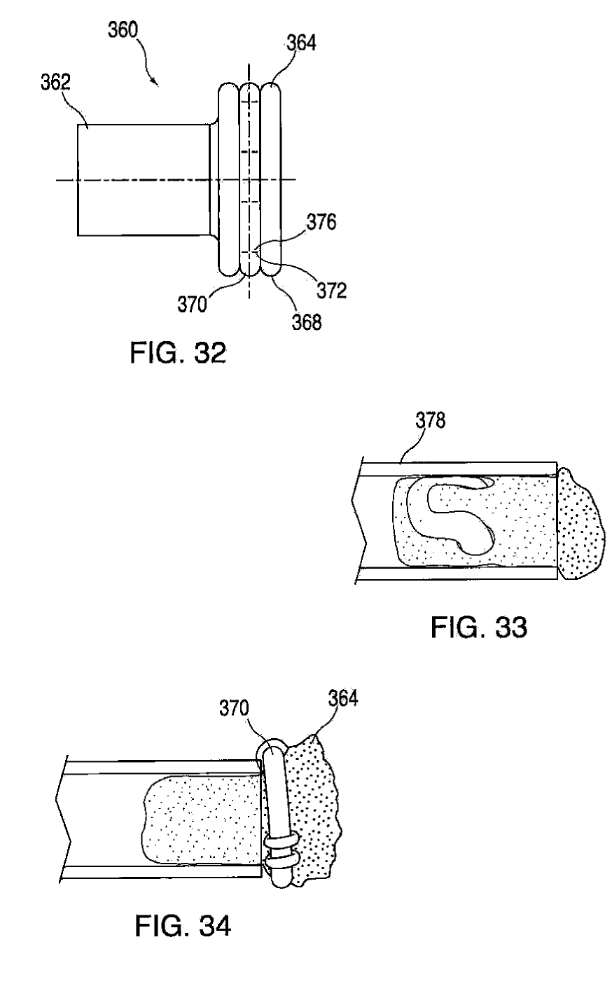

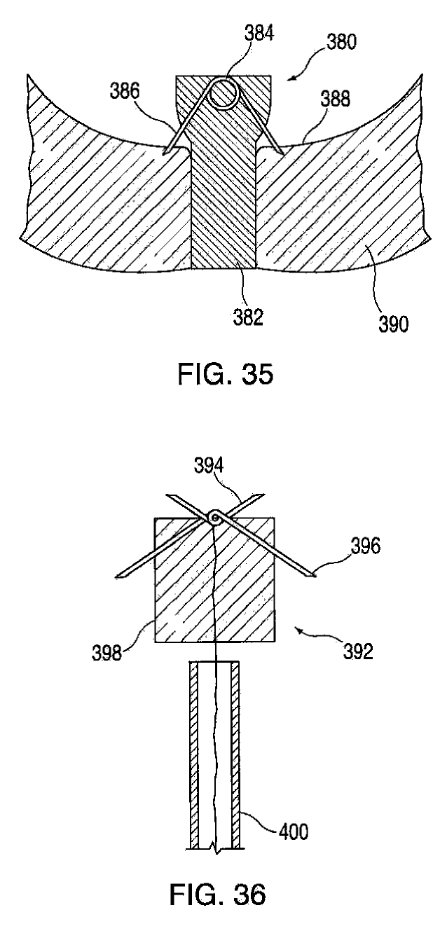

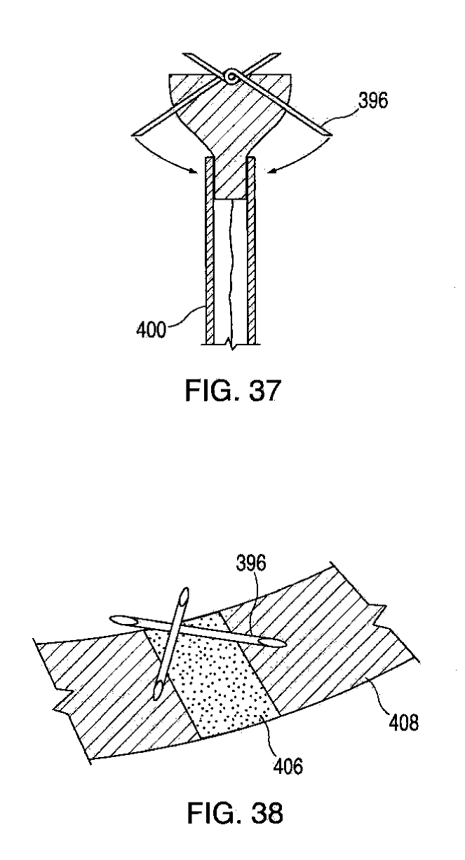

In another embodiment of the invention shown in the lateral view of FIG. 32, an implant 360 has a cylindrical base 362 and a disk-shaped distal member 364. Distal member 364 has an outer surface 368 in which a ring member 370 is positioned. Ring member 370 is attached to surface 368 by sutures 372 or other means that either loop around ring member 370 or pass through holes 376 in ring member 370.

Ring member 370 comprises a material such as rubber that is less compressible than the foam of base 362 and distal member 364. When implant 360 is delivered through a sheath 378, base 362 and distal member 364 will compress, as shown in FIG. 33. As implant 360 is advanced distally, as shown in FIG. 34, disk-shaped member 364 expands to fill out ring member 370, which would engage the inner surface of an annulus (not shown).

An embodiment of the invention known as the “pigtail” is shown in partial cross-section in FIG. 35, wherein an implant 380 has a cylindrical base member 382 similar in shape to cylindrical base member 130. A coil spring 384 is positioned within base member 382. Implant 380 is advanced through a delivery sheath (not shown), whereupon the arms 386 of spring 384 open to engage the inner surface 388 of annulus 390.

The implant 392 of FIG. 36 has crossed barbs 394 that have tips 396 that extend beyond the outer lateral surface 398 of implant 392 in an uncompressed state. When implant 392 is positioned in a delivery sheath, trocar, cannula, or endoscope 400, as shown in FIG. 37, barb tips 396 are rotated proximally to fit delivery sheath 400. After delivery sheath 400 is positioned in an annulus 404 and delivery sheath 400 is withdrawn proximally, as shown in FIG. 40, barb tips 396 engage the wall 406 of annulus 408.

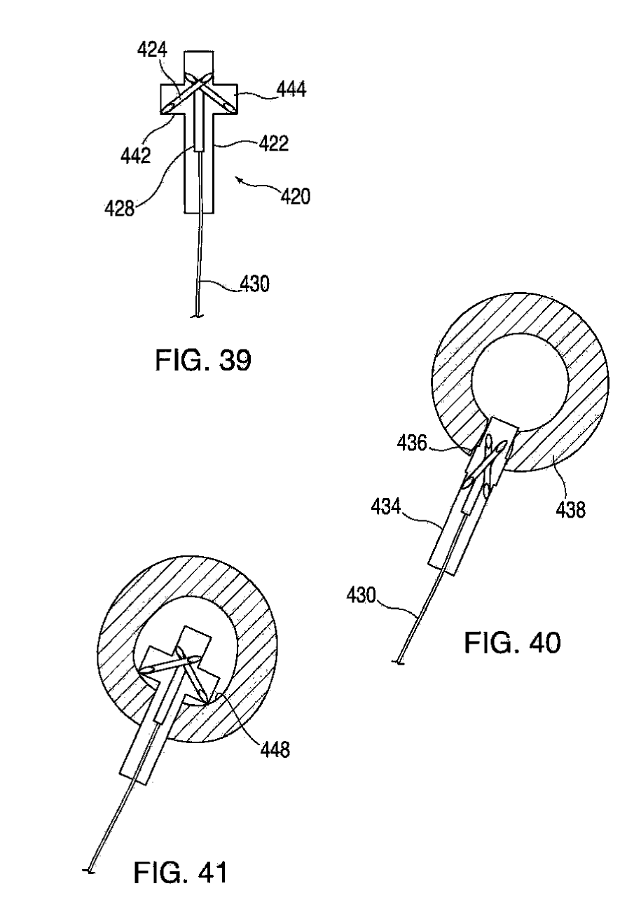

Another embodiment of the invention is shown in FIG. 39, where an implant 420 comprises a base member 422, comparable to base member 144, that has a cross-hinged member 424 that is substantially internal to implant 420. Member 424 comprises a proximally extending stem 428 that optionally has a suture 430 affixed thereto. Implant 420 is shown in FIG. 40 in a compressed state within a delivery sheath, trocar, cannula, or endoscope 434 that has been positioned within an opening 436 of an annulus 438. When delivery sheath 434 is withdrawn proximally, implant 420 expands so that the proximal surface 442 of a cylindrical member 444 engages the inner surface 448 of annulus 438, as shown in FIG. 41.

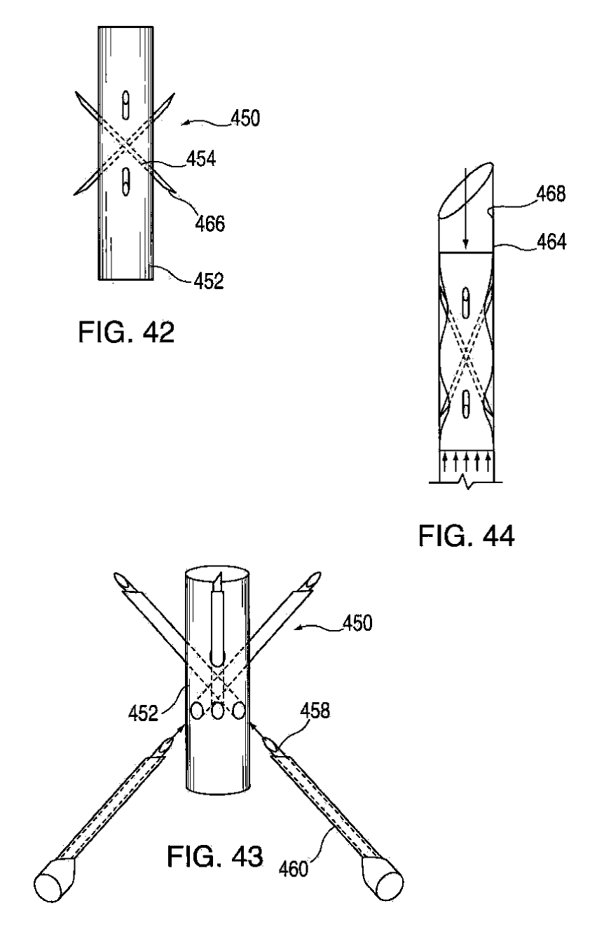

In another embodiment of the invention shown in FIG. 42, an implant 450 comprises a cylindrical member 452 with a multitude of barb elements 454, where the insertion angle can be adjusted. Implant 450 could be fabricated, for example, as shown in FIG. 43, where needles 458 are inserted through IV cannula 460 at a desired angle into the polymeric cylindrical base 452 of implant 450.

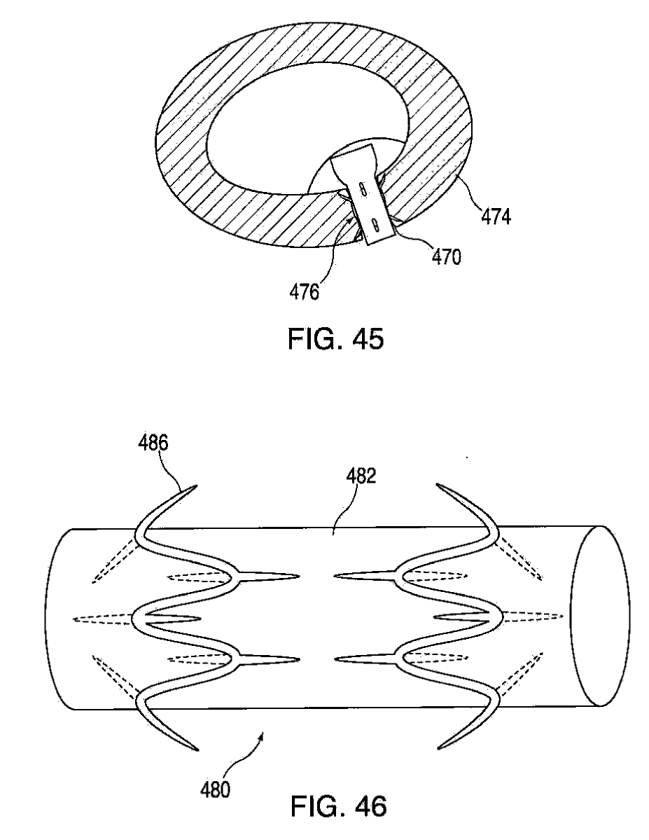

In FIG. 44, a section of a delivery sheath, trocar, cannula, or endoscope 464 comprises implant 450 where the distal tips 466 of the barb elements 454 are constrained by an inner surface 468. When, as shown in FIG. 45, delivery sheath 464 is positioned within an opening 470 of an annulus 474 and then removed, distal tips 466 engage the inner wall 476 of annulus 476.

In another embodiment of the invention shown in FIG. 46, an implant 480 comprises a cylindrical polymeric base 482 and one or more, preferably 2, annular barb-containing, stent-like members 486. Implant 480 can be compressed into a sheath, trocar, cannula, or endoscope (not shown) for delivery and then positioned to engage an annular wall (not shown).

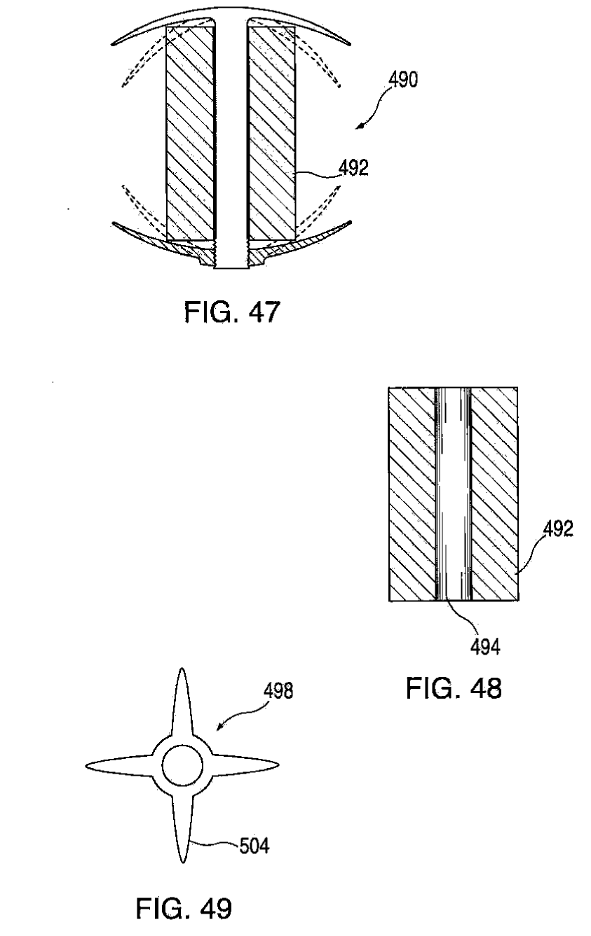

A dual anchor embodiment of the invention is shown in FIGS. 47 to 52, where an implant 490 comprises a cylindrical member 492 having a lumen 494, through which a rod member 496 extends. Rod member 496 has a flexible anchor member 498 at each end 500. Each anchor member 498 has at least 2, preferably from 2 to 4, projections 504. Each anchor member 498 can each be snapped or threaded to a rod member end 420, or one anchor member 498 could be fixedly attached to rod member 496 where the other anchor member 498 is removably attached.

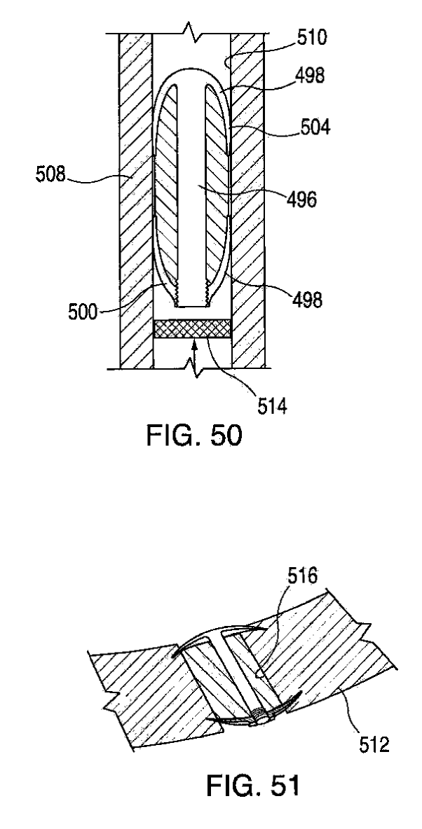

Implant 490 is shown in FIG. 50 within a delivery sheath trocar, cannula, or endoscope 508, where projections 504 are compressed against the inner surface 510 of delivery sheath 508. After delivery sheath 508 is positioned in an annulus 512 and pusher member 514 pushes implant 490 distally, projections 504 engage the wall 516 of annulus 512, as shown in FIG. 51.

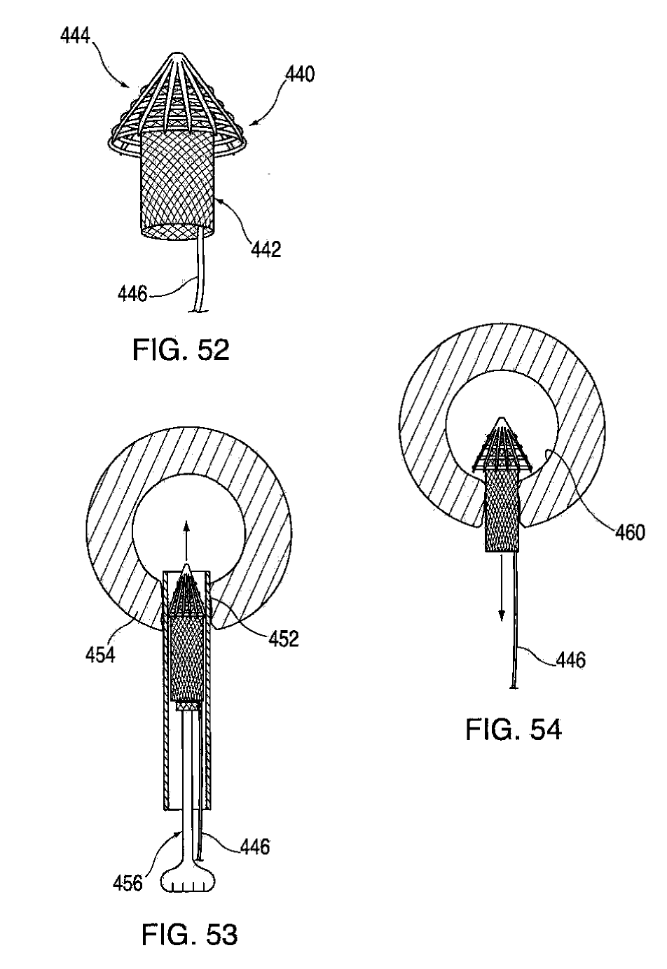

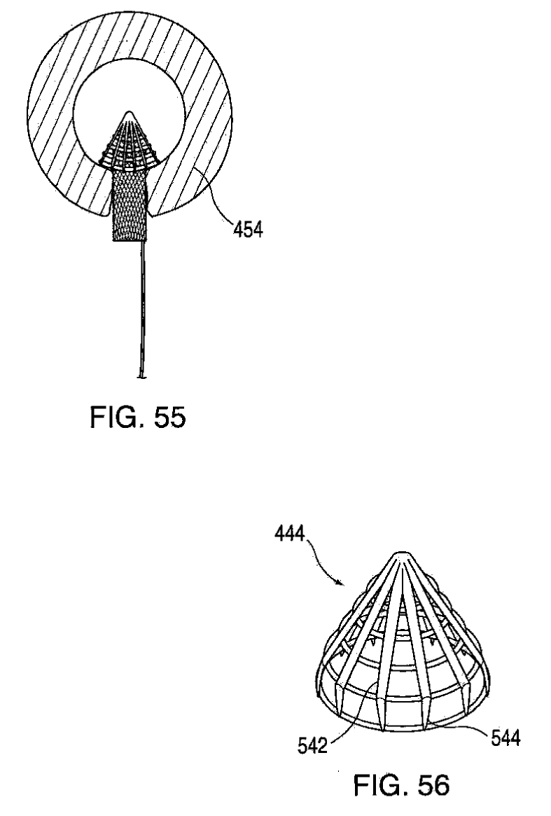

In the embodiment of the invention shown in FIG. 52, an implant 520 comprises a cylindrical elastomeric member 522, a distal conical member 524, and optionally a suture 526. Conical member 524 preferably comprises a polymeric mesh that can be compressed or closed when implant 520 is positioned within a delivery sheath, trocar, cannula, endoscope 528, as shown in FIG. 53. The distal end 532 of delivery sheath 528 is positioned within an annulus 534, and then pusher 536 advances implant 520 distally. After conical member 524 expands, suture 526 can be pulled proximally so that conical member 524 engages the inner surface 540 of annulus 534, as shown in FIGS. 54 and 55.

In the detail of conical member 444 shown in FIG. 56, it can be seen that conical member 524 may optionally have ribs 542 with soft and flexible tips 544.

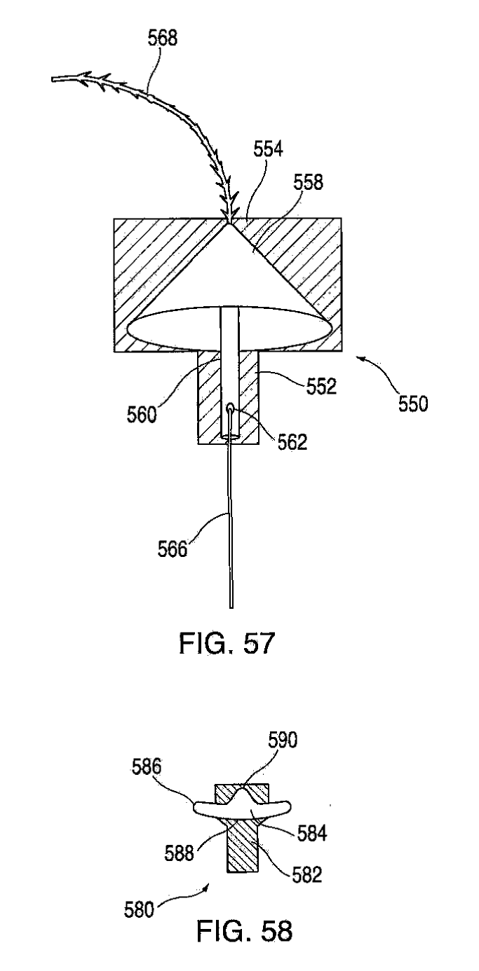

In another embodiment of the invention shown in FIG. 57, an implant 550 comprises a cylindrical base member 552 and a fixation structure 554. Fixation structure 554 comprises a conical member 558 with a proximally extending stem member 560 with a hole 562 to engage a control suture 566. Extending distally from conical member 558 is a barbed anchor suture 568 such as is available, for example, as APTOS® threads from Prollenium Medical Technologies, Ontario, Canada.

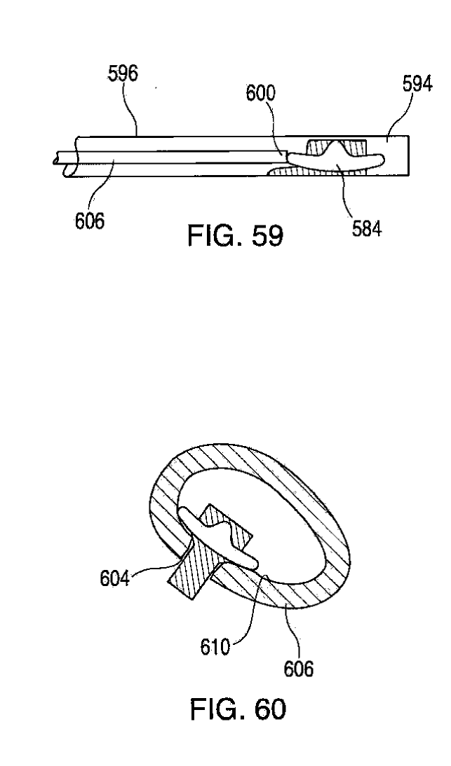

In another embodiment of the invention shown in FIG. 60, an implant 580 comprises a foam structure 582 having a crescent-shape resorbable anchor 584. Typically anchor member 584 will measure from about 12 to about 18 mm from tip 586 to tip 586 and have a dimension from its curved surface 588 to its top 590 of from about 4 to about 6 mm. Implant 580 is shown in a delivery configuration in FIG. 59, where anchor member 584 has been rotated 908 so that it can fit within the lumen 594 of a delivery sheath, trocar, cannula, or endoscope 596. The distal portion 600 of push rod 606 pushes a tip 586 of anchor member 584 to advance implant 580 distally.

As shown in FIG. 60, where implant 584 has been positioned in opening 604 in an annulus 606, the curved surface 588 and tips 586 of anchor member 584 contact the inner wall 610 of annulus 606.

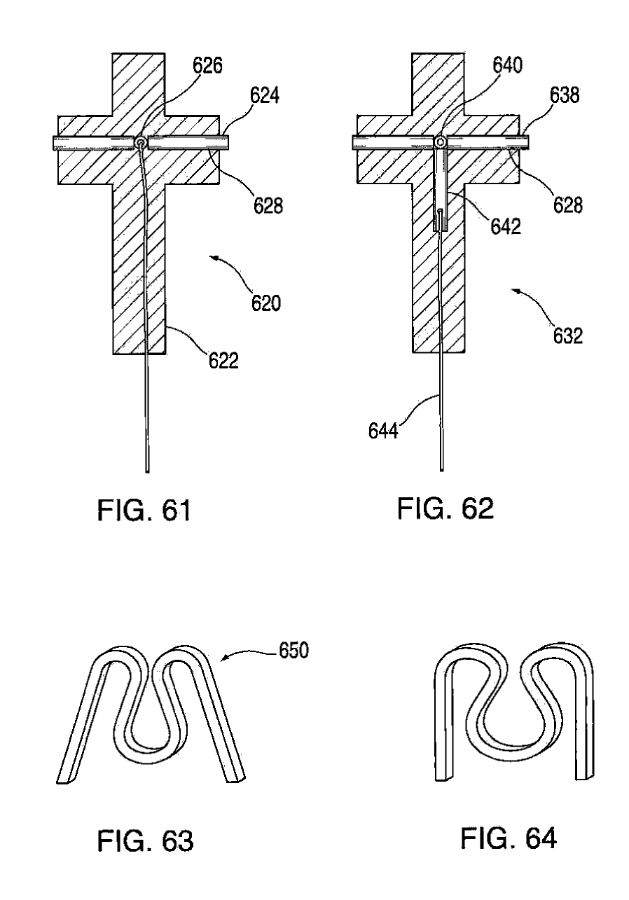

In the embodiment of the invention shown in FIG. 61, an implant 620 comprises a cylindrical base member 622 and a rod 624 having a hinge 626. A suture member 628 extends proximally from hinge 626, and consistent with delivery techniques for other embodiments, the side members 628 of rod 624 would be rotated in the proximal direction to enable implant 620 to be inserted into a delivery sheath, trocar, cannula, or endoscope (not shown). Then, implant 620 would be pushed out the distal end of the sheath, trocar or cannula to position implant 620 in an opening in an annulus (not shown).

In the embodiment of the invention shown in FIG. 62, an implant 632 comprises a cylindrical base member 634 and a rod 638 having a hinge 640 and a proximally extending stem member 642. Stem member 642 has at least one hole to engage a suture 644. Delivery and positioning in an opening in an annulus for implant 632 would be essentially the same as for implant 620.

In the aspect of the invention of FIG. 63, a wavy fixation structure 650 is shown, which structure is intended to be positioned within the mushroom portion of a cylindrical base member such as base member 130. Structure 650 can be compressed as shown in FIG. 64 for delivery within an implant, and then, once the implant is ejected from the distal portion of a delivery sheath, trocar, cannula, or endoscope, structure 650 will expand to assume its deployed or uncompressed configuration.

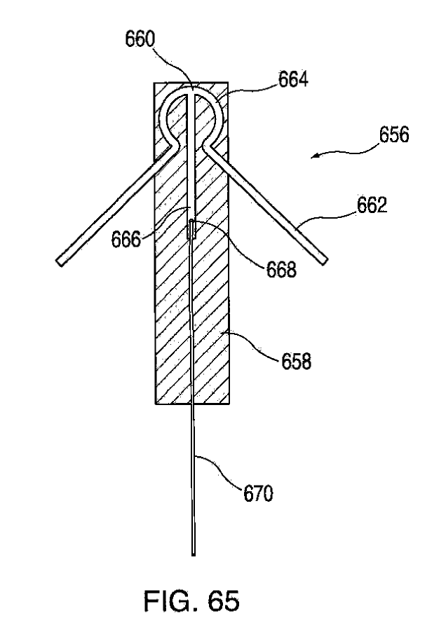

In the embodiment of the invention shown in FIG. 65, an implant 656 comprises a cylindrical base member 658 and a ribbon design fixation structure 660 having outwardly extending arms 662 and a central C-shaped section 664. Extending proximally from section 664 is a stem member 666, which optimally has at least one hold 668 to engage a suture 670.

As with other embodiments of the invention, arms 662 would be rotated in the proximal direction to enable implant 656 to be inserted into a delivery sheath, trocar, cannula, or endoscope (not shown). Then, upon delivery to the intended site in an annulus, the arm members would extend outward and be pulled back to engage the inner wall of the annulus. Implant 174 can act as a retention member or component being resistive to expulsion from the spinal annular tissue and in one embodiment will be separate from base member.

The inventive implantable device is reticulated, i.e., comprises an interconnected network of pores and channels and voids that provides fluid permeability throughout the implantable device and permits cellular and tissue ingrowth and proliferation into the interior of the implantable device. In one embodiment, the reticulated structure allows for ingrowth for such tissues as fibrous tissue and/or natural fibrous tissues. In another embodiment, the reticulated structure allows for ingrowth for such tissues as fibrovascular tissues, fibroblasts, fibrocartilage cells, endothelial tissues, etc. The tissue ingrowth can be from autologous or heterologous tissue ingrowth. In another embodiment, the tissue ingrowth and proliferation into the interior of the implantable device allows for bio-integration of the device to the site where the device is placed. In another embodiment, the tissue ingrowth and proliferation into the interior of the implantable device allows for at least partial regeneration of the device to the site where the device is placed. The inventive implantable device is reticulated, i.e., comprises an interconnected and/or inter-communicating network of pores and channels and voids that provides fluid permeability throughout the implantable device and permits cellular and tissue ingrowth and proliferation into the interior of the implantable device. The inventive implantable device is reticulated, i.e., comprises an interconnected and/or inter-communicating network of pores and/or voids and/or channels that provides fluid permeability throughout the implantable device and permits cellular and tissue ingrowth and proliferation into the interior of the implantable device. The biodurable elastomeric matrix or material is considered to be reticulated because its microstructure or the interior structure comprises inter-connected and inter-communicating pores and/or voids bounded by configuration of the struts and intersections that constitute the solid structure. The continuous interconnected void phase is the principle feature of a reticulated structure.

In aspect of the invention, the implantable device comprises substantially of a biodurable reticulated elastomeric matrix. In another aspect of the invention, the base member of the implantable device comprises substantially of a biodurable reticulated elastomeric matrix. In one embodiment, the implantable device substantially comprises of two or more reticulated elastomeric matrices having different properties. In another embodiment, the base member of the implantable device substantially comprises of two or more reticulated elastomeric matrices having different properties.

Preferred scaffold materials for the implants have a reticulated structure with sufficient and required liquid permeability and thus selected to permit blood, or other appropriate bodily fluid, and cells and tissues to access interior surfaces of the implants. This happens due to the presence of inter-connected and inter-communicating, reticulated open pores and/or voids and/or channels that form fluid passageways or fluid permeability providing fluid access all through. Over time, the tissue ingrowth and proliferation into the interior of the implantable device placed at the defect site leads to regeneration and or bio-integration of the device to the site where the device is placed. The biodurable reticulated elastomeric material that comprises the implant device will allow for tissue ingrowth and proliferation and bio-integrate the implant device to the annular defect. The tissue ingrowth and proliferation is expected to provide resistive force to prevent expulsion from the spinal annular tissue. The biodurable reticulated elastomeric material that comprises the implant device allows for tissue ingrowth from the annulus and from the surrounding tissue and will seal the annular defect and in one embodiment provide a permanent sealing of the aperture.

Preferred materials are at least partially hydrophobic reticulated, elastomeric polymeric matrix for fabricating implants according to the invention are flexible and resilient in recovery, so that the implants are also compressible materials enabling the implants to be compressed and, once the compressive force is released, to then recover to, or toward, substantially their original size and shape. For example, an implant can be compressed from a relaxed configuration or a size and shape to a compressed size and shape under ambient conditions, e.g., at 25° C. to fit into the introducer instrument for insertion into the annular defect or aperature or fissure. Alternatively, an implant may be supplied to the medical practitioner performing the implantation operation, in a compressed configuration, for example, contained in a package, preferably a sterile package. The resiliency of the elastomeric matrix that is used to fabricate the implant causes it to recover to a working size and configuration in situ, at the implantation site, after being released from its compressed state within the introducer instrument. The working size and shape or configuration can be substantially similar to original size and shape after the in situ recovery.

Preferred scaffolds are reticulated elastomeric polymeric materials having sufficient structural integrity and durability to endure the intended biological environment, for the intended period of implantation. For structure and durability, at least partially hydrophobic polymeric scaffold materials are preferred although other materials may be employed if they meet the requirements described herein. Useful materials are preferably elastomeric in that they can be compressed and can resiliently recover to substantially the pre-compression state. Alternative reticulated polymeric materials with interconnected pores or networks of pores that permit biological fluids to have ready access throughout the interior of an implant may be employed, for example, woven or nonwoven fabrics or networked composites of microstructural elements of various forms.

A partially hydrophobic scaffold is preferably constructed of a material selected to be sufficiently biodurable, for the intended period of implantation that the implant will not lose its structural integrity during the implantation time in a biological environment. The biodurable elastomeric matrices forming the scaffold do not exhibit significant symptoms of breakdown, degradation, erosion or significant deterioration of mechanical properties relevant to their use when exposed to biological environments and/or bodily stresses for periods of time commensurate with the use of the implantable device. In one embodiment, the desired period of exposure is to be understood to be at least 29 days, preferably several weeks and most preferably 2 to 5 years or more. This measure is intended to avoid scaffold materials that may decompose or degrade into fragments, for example, fragments that could have undesirable effects such as causing an unwanted tissue response.

The void phase, preferably continuous and interconnected, of the reticulated polymeric matrix that is used to fabricate the implant of this invention may comprise as little as 50% by volume of the elastomeric matrix, referring to the volume provided by the interstitial spaces of elastomeric matrix before any optional interior pore surface coating or layering is applied. In one embodiment, the volume of void phase as just defined, is from about 70% to about 99% of the volume of elastomeric matrix. In another embodiment, the volume of void phase is from about 80% to about 98% of the volume of elastomeric matrix. In another embodiment, the volume of void phase is from about 90% to about 98% of the volume of elastomeric matrix.

As used herein, when a pore is spherical or substantially spherical, its largest transverse dimension is equivalent to the diameter of the pore. When a pore is non-spherical, for example, ellipsoidal or tetrahedral, its largest transverse dimension is equivalent to the greatest distance within the pore from one pore surface to another, e.g., the major axis length for an ellipsoidal pore or the length of the longest side for a tetrahedral pore. Scanning electron micrograph (SEM) images of the reticulated elastomeric matrix demonstrated, e.g., the network of cells interconnected via the open pores therein. The average pore diameter or other largest transverse dimension of the pores of the reticulated elastomeric matrix and can be determined from SEM observations. For those skilled in the art, one can routinely estimate the pore frequency per unit length and further estimate the average pore diameter in microns. When using optical microscopy technique, the average cell diameter or other largest transverse dimension of the reticulated elastomeric matrix is determined and the cell diameter is a more a measure of the 3 dimensional superstructure are interconnected via the open pores.

In one embodiment relating to orthopedic and spinal implant applications and the like, to encourage cellular ingrowth and proliferation and to provide adequate fluid permeability, the average diameter or other largest transverse dimension of pores is at least about 20 μm. In another embodiment, the average diameter or other largest transverse dimension of pores is at least about 50 μm. In another embodiment, the average diameter or other largest transverse dimension of pores is at least about 100 μm. In another embodiment, the average diameter or other largest transverse dimension of pores is at least about 150 μm. In another embodiment, the average diameter or other largest transverse dimension of pores is at least about 250 μm. In another embodiment, the average diameter or other largest transverse dimension of pores is greater than about 250 μm. In another embodiment, the average diameter or other largest transverse dimension of pores is greater than 250 μm. In another embodiment, the average diameter or other largest transverse dimension of pores is at least about 275 μm. In another embodiment, the average diameter or other largest transverse dimension of pores is greater than about 275 μm. In another embodiment, the average diameter or other largest transverse dimension of pores is greater than 275 μm. In another embodiment, the average diameter or other largest transverse dimension of pores is at least about 300 μm. In another embodiment, the average diameter or other largest transverse dimension of pores is greater than about 300 μm. In another embodiment, the average diameter or other largest transverse dimension of pores is greater than 300 μm.

In another embodiment relating to orthopedic and spinal implant applications and the like, the average diameter or other largest transverse dimension of pores is not greater than about 900 μm. In another embodiment, the average diameter or other largest transverse dimension of pores is not greater than about 750 μm. In another embodiment, the average diameter or other largest transverse dimension of pores is not greater than about 500 μm. In another embodiment, the average diameter or other largest transverse dimension of pores is not greater than about 400 μm. In another embodiment, the average diameter or other largest transverse dimension of pores is not greater than about 300 μm. In another embodiment, the average diameter or other largest transverse dimension of pores is not greater than about 200 μm. In another embodiment, the average diameter or other largest transverse dimension of pores is not greater than about 100 μm.

In one embodiment relating to orthopedic and spinal implant applications and the like, to encourage cellular ingrowth and proliferation and to provide adequate fluid permeability, the average diameter or other largest transverse dimension of the cell is at least about 75 μm. In another embodiment, the average diameter or other largest transverse dimension of cells is at least about 150 μm. In another embodiment, the average diameter or other largest transverse dimension of cells is at least about 250 μm. In another embodiment, the average diameter or other largest transverse dimension of cells is at least about 350 μm. In another embodiment, the average diameter or other largest transverse dimension of cells is at least about 500 μm. In another embodiment, the average diameter or other largest transverse dimension of cells is at least about 700 μm. In another embodiment, the average diameter or other largest transverse dimension of cells is at least about 1000 μm. In another embodiment, the average diameter or other largest transverse dimension of cells range from about 75 to 1000 μm. In another embodiment, the average diameter or other largest transverse dimension of cells range from about 100 to 500 μm. In another embodiment, the average diameter or other largest transverse dimension of cells range from about 150 to 300 μm.

In one embodiment, the invention comprises an implantable device having sufficient resilient compressibility to be delivered by a “delivery device”, i.e., a device with a chamber for containing an reticulated elastomeric biodurable reticulated implantable device while it is delivered to the desired site then released at the site, e.g., using a trocar, cannula, or through an endoscopic instrument such as an arthroscope, laproscope, or cystoscope. In another embodiment, the thus-delivered elastomeric biodurable reticulated implantable device substantially regains its shape after delivery to a biological site and has adequate biodurability and biocompatibility characteristics to be suitable for long-term implantation.

One embodiment for use in the practice of the invention is a reticulated elastomeric implant which is sufficiently flexible and resilient, i.e., resiliently-compressible, to enable it to be initially compressed under ambient conditions, e.g., at 25° C., from a relaxed configuration to a first, compact configuration for delivery via a delivery-device, e.g., an endoscopic instrument such as an arthroscope, laproscope, cystoscope, or endoscope, or other suitable introducer instrument such as syringe, trocar, etc., for delivery in vitro and, thereafter, to expand to a second, working configuration in situ. In another embodiment, reticulated elastomeric implant is delivered in an uncompressed state via a delivery-device. Furthermore, in another embodiment, an reticulated elastomeric matrix has the herein described resilient-compressibility after being compressed about 5-95% of an original dimension (e.g., compressed about 19/20th- 1/20th of an original dimension). In another embodiment, an reticulated elastomeric matrix has the herein described resilient-compressibility after being compressed about 10-90% of an original dimension (e.g., compressed about 9/10th- 1/10th of an original dimension). As used herein, reticulated elastomeric implant has “resilient-compressibility”, i.e., is “resiliently-compressible”, when the second, working configuration, in vitro, is at least about 30% of the size of the relaxed configuration in at least one dimension. As used herein, reticulated elastomeric implant has “resilient-compressibility”, i.e., is “resiliently-compressible”, when the second, working configuration, in vitro, is at least about 50% of the size of the relaxed configuration in at least one dimension. In another embodiment, the resilient-compressibility of reticulated elastomeric implant is such that the second, working configuration, in vitro, is at least about 80% of the size of the relaxed configuration in at least one dimension. In another embodiment, the resilient-compressibility of reticulated elastomeric implant is such that the second, working configuration, in vitro, is at least about 90% of the size of the relaxed configuration in at least one dimension. In another embodiment, the resilient-compressibility of reticulated elastomeric implant is such that the second, working configuration, in vitro, is at least about 97% of the size of the relaxed configuration in at least one dimension.

In another embodiment, a reticulated elastomeric matrix has the herein described resilient-compressibility after being compressed about 5-95% of its original volume (e.g., compressed about 19/20th- 1/20th of its original volume). In another embodiment, an reticulated elastomeric matrix has the herein described resilient-compressibility after being compressed about 10-90% of its original volume (e.g., compressed about 9/10th- 1/10th of its original volume). As used herein, “volume” is the volume swept-out by the outermost three-dimensional contour of the reticulated elastomeric matrix. In another embodiment, the resilient-compressibility of reticulated elastomeric implant is such that the second, working configuration, in vivo, is at least about 30% of the volume occupied by the relaxed configuration. In another embodiment, the resilient-compressibility of reticulated elastomeric implant is such that the second, working configuration, in vivo, is at least about 50% of the volume occupied by the relaxed configuration. In another embodiment, the resilient-compressibility of reticulated elastomeric implant is such that the second, working configuration, in vivo, is at least about 80% of the volume occupied by the relaxed configuration. In another embodiment, the resilient-compressibility of reticulated elastomeric implant is such that the second, working configuration, in vivo, is at least about 90% of the volume occupied by the relaxed configuration. In another embodiment, the resilient-compressibility of reticulated elastomeric implant is such that the second, working configuration, in vivo, occupies at least about 97% of the volume occupied by the reticulated elastomeric matrix in its relaxed configuration.

In another embodiment, a reticulated elastomeric matrix has the herein described resilient-compressibility is delivered to the target orthopedic or spinal implant but is not compressed during delivery to the target defect site. In another embodiment, after being delivered in an uncompressed state, the resilient-compressibility of reticulated elastomeric implant is such that the second working configuration, in vivo, occupies at least about 25% to at least about 40% of the volume occupied by the reticulated elastomeric matrix in its relaxed configuration. In another embodiment, after being delivered in an uncompressed state, the resilient-compressibility of reticulated elastomeric implant is such that the second working configuration, in vivo, occupies at least about 40% to at least about 80% of the volume occupied by the reticulated elastomeric matrix in its relaxed configuration. In another embodiment, after being delivered in an uncompressed state, the resilient-compressibility of reticulated elastomeric implant is such that the second working configuration, in vivo, occupies at least about 80% to at least about 95% of the volume occupied by the reticulated elastomeric matrix in its relaxed configuration. In another embodiment, after being delivered in an uncompressed state, the resilient-compressibility of reticulated elastomeric implant is such that the second working configuration, in vivo, occupies at least about 95% to at least about 98% of the volume occupied by the reticulated elastomeric matrix in its relaxed configuration. In another embodiment, after being delivered in an uncompressed state, the resilient-compressibility of reticulated elastomeric implant is such that the second working configuration, in vivo, occupies the entire volume occupied by the reticulated elastomeric matrix in its relaxed configuration.

It is contemplated, in another embodiment, that upon implantation, before their pores become filled with biological fluids, bodily fluids and/or tissue, such implantable devices for orthopedic applications and the like do not entirely fill, cover or span the biological site in which they reside and that an individual implanted reticulated elastomeric matrix will, in many cases although not necessarily, have at least one dimension of no more than 75% of the biological site within the entrance thereto or over 75% of the damaged tissue that is being repaired or replaced. In another embodiment, an individual implanted reticulated elastomeric matrix as described above will have at least one dimension of no more than 95% of the biological site within the entrance thereto or over 95% of the damaged tissue that is being repaired or replaced.

In another embodiment, that upon implantation, before their pores become filled with biological fluids, bodily fluids and/or tissue, such implantable devices for orthopedic applications and the like substantially fill, cover or span the biological site in which they reside and an individual implanted reticulated elastomeric matrix will, in many cases, although not necessarily, have at least one dimension of no more than about 98% of the biological site within the entrance thereto or cover 98% of the damaged tissue that is being repaired or replaced. In another embodiment, an individual implanted reticulated elastomeric matrix as described above will have at least one dimension of no more than about 100% of the biological site within the entrance thereto or cover 100% of the damaged tissue that is being repaired or replaced. In another embodiment, an individual implanted reticulated elastomeric matrix as described above will have at least one dimension of no more than about 102% of the biological site within the entrance thereto or cover 102% of the damaged tissue that is being repaired or replaced.

In another embodiment, that upon implantation, before their pores become filled with biological fluids, bodily fluids and/or tissue, such implantable devices for orthopedic applications and the like overfill, cover or span the biological site in which they reside and an individual implanted reticulated elastomeric matrix will, in many cases, although not necessarily, have at least one dimension of more than about 125% of the biological site within the entrance thereto or cover 125% of the damaged tissue that is being repaired or replaced. In another embodiment, an individual implanted reticulated elastomeric matrix as described above will have at least one dimension of more than about 200% of the biological site within the entrance thereto or cover 200% of the damaged tissue that is being repaired or replaced. In another embodiment, an individual implanted reticulated elastomeric matrix as described above will have at least one dimension of more than about 150% of the biological site within the entrance thereto or cover 150% of the damaged tissue that is being repaired or replaced. In another embodiment, an individual implanted reticulated elastomeric matrix as described above will have at least one dimension of more than about 200% of the biological site within the entrance thereto or cover 200% of the damaged tissue that is being repaired or replaced. In another embodiment, an individual implanted reticulated elastomeric matrix as described above will have at least one dimension of more than about 300% of the biological site within the entrance thereto or cover 300% of the damaged tissue that is being repaired or replaced.

The reticulated elastomeric matrix useful according to the invention should have sufficient mechanical integrity reflected for example in tensile and compressive properties such that it can withstand normal manual or mechanical handling during its intended application and during post-processing steps that may be required or desired without tearing, breaking, crumbling, fragmenting or otherwise disintegrating, shedding pieces or particles, or otherwise losing its structural integrity. The tensile and compressive properties of the matrix material(s) should not be so high as to interfere with the fabrication or other processing of the reticulated elastomeric matrix. The tensile and compressive properties should be appropriate so that they can withstand the forces, loads, deformations and moments experienced by the implant when placed at the target orthopedic or spinal implant site. In one embodiment, the reticulated elastomeric matrix has sufficient structural integrity to be self-supporting and free-standing in vitro. However, in another embodiment, the elastomeric matrix can be furnished with structural supports such as ribs or struts.

In one embodiment, the reticulated polymeric matrix that is used to fabricate the implants of this invention has any suitable bulk density, also known as specific gravity, consistent with its other properties. For example, in one embodiment, the bulk density may be from about 0.005 to about 0.15 g/cc (from about 0.31 to about 9.4 lb/ft³), preferably from about 0.016 to about 0.136 g/cc (from about 1.0 to about 8.5 lb/ft³) and most preferably from about 0.032 to about 0.136 g/cc (from about 2.0 to about 8.5 lb/ft³).

The reticulated elastomeric matrix has sufficient tensile strength such that it can withstand normal manual or mechanical handling during its intended application and during post-processing steps that may be required or desired without tearing, breaking, crumbling, fragmenting or otherwise disintegrating, shedding pieces or particles, or otherwise losing its structural integrity. The tensile strength of the starting material(s) should not be so high as to interfere with the fabrication or other processing of elastomeric matrix. Thus, for example, in one embodiment, the reticulated polymeric matrix that is used to fabricate the implants of this invention may have a tensile strength of from about 700 to about 140,000 kg/m² (from about 1 to about 200 psi). In another embodiment, elastomeric matrix may have a tensile strength of from about 14,050 to about 70,300 kg/m² (from about 20 to about 100 psi). In another embodiment, elastomeric matrix may have a tensile strength of from about 1,400 to about 28,000 kg/m² (from about 2 to about 40 psi) at 20% ultimate tensile elongation strain. Sufficient ultimate tensile elongation is also desirable. For example, in another embodiment, reticulated elastomeric matrix has an ultimate tensile elongation of at least about 50% to at least about 400%. In yet another embodiment, reticulated elastomeric matrix has an ultimate tensile elongation of at least 70% to at least about 300%.

In one embodiment, reticulated elastomeric matrix that is used to fabricate the implants of this invention has a compressive strength of from about 700 to about 70,000 kg/m² (from about 1.0 to about 100 psi) at 50% compression strain. In another embodiment, reticulated elastomeric matrix has a compressive strength of from about 700 to about 140,000 kg/m² (from about 1 to about 200 psi) at 75% compression strain.

In another embodiment, reticulated elastomeric matrix that is used to fabricate the implants of this invention has a compression set, when compressed to 50% of its thickness at about 25° C., of not more than about 30%. In another embodiment, reticulated elastomeric matrix has a compression set of not more than about 20%. In another embodiment, reticulated elastomeric matrix has a compression set of not more than about 10%. In another embodiment, reticulated elastomeric matrix has a compression set of not more than about 5%. In one embodiment, the elastomeric matrix expands from the first, compact configuration to the second, working configuration over a short time, e.g., about 95% recovery in 90 seconds or less in one embodiment, or in 40 seconds or less in another embodiment, each from 75% compression strain held for up to 10 minutes. In another embodiment, the expansion from the first, compact configuration to the second, working configuration occurs over a short time, e.g., about 95% recovery in 180 seconds or less in one embodiment, in 90 seconds or less in another embodiment, in 60 seconds or less in another embodiment, each from 75% compression strain held for up to 30 minutes. In another embodiment, elastomeric matrix recovers in about 10 minutes to occupy at least about 97% of the volume occupied by its relaxed configuration, following 75% compression strain held for up to 30 minutes.

In another embodiment, reticulated elastomeric matrix that is used to fabricate the implants of this invention has a tear strength, of from about 0.18 to about 3.6 kg/linear cm (from about 1 to about 20 lbs/linear inch).

In another embodiment of the invention the reticulated elastomeric matrix that is used to fabricate the implant can be readily permeable to liquids, permitting flow of liquids, including blood, through the composite device of the invention. The water permeability of the reticulated elastomeric matrix is from about 30 to about 500 Darcy, preferably from about 50 to about 300 Darcy. In contrast, permeability of the unreticulated elastomeric matrix is below about 1 Darcy. In another embodiment, the permeability of the unretriculated elastomeric matrix is below Darcy.

In general, suitable biodurable reticulated elastomeric partially hydrophobic polymeric matrix that is used to fabricate the implant of this invention or for use as scaffold material for the implant in the practice of the present invention, in one embodiment sufficiently well characterized, comprise elastomers that have or can be formulated with the desirable mechanical properties described in the present specification and have a chemistry favorable to biodurability such that they provide a reasonable expectation of adequate biodurability.

Various biodurable reticulated hydrophobic polyurethane materials are suitable for this purpose. In one embodiment, structural materials for the inventive reticulated elastomers are synthetic polymers, especially, but not exclusively, elastomeric polymers that are resistant to biological degradation, for example, polycarbonate polyurethane-urea, polycarbonate polyurea-urethane, polycarbonate polyurethane, polycarbonate polysiloxane polyurethane, and polysiloxane polyurethane, and the like. Such elastomers are generally hydrophobic but, pursuant to the invention, may be treated to have surfaces that are less hydrophobic or somewhat hydrophilic. In another embodiment, such elastomers may be produced with surfaces that are less hydrophobic or somewhat hydrophilic.

The invention can employ, for implanting, a biodurable reticulatable elastomeric partially hydrophobic polymeric scaffold material or matrix for fabricating the implant or a material. More particularly, in one embodiment, the invention provides a biodurable elastomeric polyurethane scaffold material or matrix which is made by synthesizing the scaffold material or matrix preferably from a polycarbonate polyol component and an isocyanate component by polymerization, cross-linking and foaming, thereby forming pores, followed by reticulation of the porous material to provide a biodurable reticulated elastomeric product with inter-connected and/or inter-communicating pores and channels. The product is designated as a polycarbonate polyurethane, being a polymer comprising urethane groups formed from, e.g., the hydroxyl groups of the polycarbonate polyol component and the isocyanate groups of the isocyanate component. In another embodiment, the invention provides a biodurable elastomeric polyurethane scaffold material or matrix which is made by synthesizing the scaffold material or matrix preferably from a polycarbonate polyol component and an isocyanate component by polymerization, cross-linking and foaming, thereby forming pores, and using water as a blowing agent and/or foaming agent during the synthesis, followed by reticulation of the porous material to provide a biodurable reticulated elastomeric product with inter-connected and/or inter-communicating pores and channels. This product is designated as a polycarbonate polyurethane-urea or polycarbonate polyurea-urethane, being a polymer comprising urethane groups formed from, e.g., the hydroxyl groups of the polycarbonate polyol component and the isocyanate groups of the isocyanate component and also comprising urea groups formed from reaction of water with the isocyanate groups. In all of these embodiments, the process employs controlled chemistry to provide a reticulated elastomeric matrix or product with good biodurability characteristics. The matrix or product employing chemistry that avoids biologically undesirable or nocuous constituents therein.

In one embodiment, the starting material for synthesizing the biodurable reticulated elastomeric partially hydrophobic polymeric matrix contains at least one polyol component to provide the so-called soft segment. For the purposes of this application, the term “polyol component” includes molecules comprising, on the average, about 2 hydroxyl groups per molecule, i.e., a difunctional polyol or a diol, as well as those molecules comprising, on the average, greater than about 2 hydroxyl groups per molecule, i.e., a polyol or a multi-functional polyol. In one embodiment, this soft segment polyol is terminated with hydroxyl groups, either primary or secondary. Exemplary polyols can comprise, on the average, from about 2 to about 5 hydroxyl groups per molecule. In one embodiment, as one starting material, the process employs a difunctional polyol component in which the hydroxyl group functionality of the diol is about 2. In another embodiment, the soft segment is composed of a polyol component that is generally of a relatively low molecular weight, typically from about 500 to about 6,000 Daltons and preferably between 1000 to 2500 Daltons. Examples of suitable polyol components include but not limited to polycarbonate polyol, hydrocarbon polyol, polysiloxane polyol, poly(carbonate-co-hydrocarbon) polyol, poly(carbonate-co-siloxane) polyol, poly(hydrocarbon-co-siloxane) polyol, polysiloxane polyol and copolymers and mixtures thereof.

In one embodiment, the starting material for synthesizing the biodurable reticulated elastomeric partially hydrophobic polymeric matrix contains at least one isocyanate component and, optionally, at least one chain extender component to provide the so-called “hard segment”. In one embodiment, the starting material for synthesizing the biodurable reticulated elastomeric partially hydrophobic polymeric matrix contains at least one isocyanate component. For the purposes of this application, the term “isocyanate component” includes molecules comprising, on the average, about 2 isocyanate groups per molecule as well as those molecules comprising, on the average, greater than about 2 isocyanate groups per molecule. The isocyanate groups of the isocyanate component are reactive with reactive hydrogen groups of the other ingredients, e.g., with hydrogen bonded to oxygen in hydroxyl groups of the polyol component, with hydrogen bonded to nitrogen in amine groups, chain extender, cross-linker and/or water. In one embodiment, the average number of isocyanate groups per molecule in the isocyanate component is about 2. In another embodiment, the average number of isocyanate groups per molecule in the isocyanate component is greater than about 2.

The isocyanate index, a quantity well known to those in the art, is the mole ratio of the number of isocyanate groups in a formulation available for reaction to the number of groups in the formulation that are able to react with those isocyanate groups, e.g., the reactive groups of diol(s), polyol component(s), chain extender(s) and water, when present. In one embodiment, the isocyanate index is from about 0.9 to about 1.1. In another embodiment, the isocyanate index is from about 0.9 to about 1.02. In another embodiment, the isocyanate index is from about 0.98 to about 1.02. In another embodiment, the isocyanate index is from about 0.9 to about 1.0. In another embodiment, the isocyanate index is from about 0.9 to about 0.98.

In one embodiment, a small quantity of an optional ingredient, such as a multi-functional hydroxyl compound or other cross-linker having a functionality greater than 2, is present to allow crosslinking and/or to achieve a stable foam, i.e., a foam that does not collapse to become non-foamlike. Alternatively, or in addition, polyfunctional adducts of aliphatic and cycloaliphatic isocyanates can be used to impart cross-linking in combination with aromatic diisocyanates. Alternatively, or in addition, polyfunctional adducts of aliphatic and cycloaliphatic isocyanates can be used to impart cross-linking in combination with aliphatic diisocyanates. Alternatively, or in addition, polymeric aromatic diisocyanates can be used to impart cross-linking. The presence of these components and adducts with functionality higher than 2 in the hard segment component allows for cross-linking to occur. In distinction to the cross-linking described above which is termed chemical cross-linking, additional cross-linking arises out of hydrogen bonding in and between both the hard and soft phases of the matrix and is termed as physical cross-linking.

Exemplary diisocyanates include aliphatic diisocyanates, isocyanates comprising aromatic groups, the so-called “aromatic diisocyanates”, and mixtures thereof. Aliphatic diisocyanates include tetramethylene diisocyanate, cyclohexane-1,2-diisocyanate, cyclohexane-1,4-diisocyanate, hexamethylene diisocyanate, isophorone diisocyanate, methylene-bis-(p-cyclohexyl isocyanate) (“H12 MDI”), and mixtures thereof. Aromatic diisocyanates include p-phenylene diisocyanate, 4,4′-diphenylmethane diisocyanate (“4,4′-MDI”), 2,4′-diphenylmethane diisocyanate (“2,4′-MDI”), polymeric MDI, and mixtures thereof. Examples of optional chain extenders include diols, diamines, alkanol amines or a mixture thereof.

In one embodiment, the starting material for synthesizing the biodurable reticulated elastomeric partially hydrophobic polymeric matrix contains at least one blowing agent such as water. Other exemplary blowing agents include the physical blowing agents, e.g., volatile organic chemicals such as hydrocarbons, ethanol and acetone, and various fluorocarbons, hydrofluorocarbons, chlorofluorocarbons, and hydrochlorofluorocarbons. Additional exemplary blowing agents include the physical blowing agents such as gases as nitrogen, helium, etc., that can additionally act as nucleating agent and whose amount and the pressure under which they are introduced during matrix formation can be used to control the density of the biodurable, elastomeric and partially hydrophobic polymeric matrix. In one embodiment, the hard segments also contain a urea component formed during foaming reaction with water. In one embodiment, the reaction of water with an isocyanate group yields carbon dioxide, which serves as a blowing agent. The amount of blowing agent, e.g., water, is adjusted to obtain different densities of non-reticulated foams. A reduced amount of blowing agent such as water may reduce the number of urea linkages in the material.

In one embodiment, implantable device can be rendered radiopaque to facilitate in vivo imaging, for example, by adhering to, covalently bonding to and/or incorporating into the elastomeric matrix itself particles of a radio-opaque material. Radio-opaque materials include titanium, tantalum, tungsten, barium sulfate or other suitable material known to those skilled in the art. In addition to incorporating radiopaque agents such as tantalum into the implant material itself, a further embodiment of the invention encompasses the use of radiopaque metallic components to impart radiopacity to the implant. For example, thin filaments comprised of metals with shape memory properties such as platinum or nitinol can be embedded into the implant and may be in the form of a straight or curved wire, helical or coil-like structure, umbrella structure, or other structure generally known to those skilled in the art. Alternatively, a metallic frame around the implant may also be used to impart radiopacity. The metallic frame may be in the form of a tubular structure, a helical or coil-like structure, an umbrella structure, or other structure generally known to those skilled in the art. Attachment of radiopaque metallic components to the implant can be accomplished by means including but not limited to chemical bonding or adhesion, suturing, pressure fitting, compression fitting, and other physical methods.

In one embodiment, the starting material of the biodurable reticulated elastomeric partially hydrophobic polymeric matrix is a commercial polyurethane polymers are linear, not crosslinked, polymers, therefore, they are soluble, can be melted, readily analyzable and readily characterizable. In this embodiment, the starting polymer provides good biodurability characteristics. The reticulated elastomeric matrix is produced by taking a solution of the commercial polymer such as polyurethane and charging it into a mold that has been fabricated with surfaces defining a microstructural configuration for the final implant or scaffold, solidifying the polymeric material and removing the sacrificial mold by melting, dissolving or subliming-away the sacrificial mold. In one embodiment, the solvents can be lyophilized leaving at least a partially or fully reticulated material matrix. The matrix or product employing a foaming process that avoids biologically undesirable or nocuous constituents therein.

Of particular interest are thermoplastic elastomers such as polyurethanes whose chemistry is associated with good biodurability properties, for example. In one embodiment, such thermoplastic polyurethane elastomers include polycarbonate polyurethanes, polysiloxane polyurethanes, polyurethanes with so-called “mixed” soft segments, and mixtures thereof. Mixed soft segment polyurethanes are known to those skilled in the art and include, e.g., polycarbonate-polysiloxane polyurethanes. In another embodiment, the thermoplastic polyurethane elastomer comprises at least one diisocyanate in the isocyanate component, at least one chain extender and at least one diol, and may be formed from any combination of the diisocyanates, difunctional chain extenders and diols described in detail above. Some suitable thermoplastic polyurethanes for practicing the invention, in one embodiment suitably characterized as described herein, include: polyurethanes with mixed soft segments comprising polysiloxane together with a polycarbonate component.

In one embodiment, the weight average molecular weight of the thermoplastic elastomer is from about 30,000 to about 500,000 Daltons. In another embodiment, the weight average molecular weight of the thermoplastic elastomer is from about 50,000 to about 250,000 Daltons.

Some commercially-available thermoplastic elastomers suitable for use in practicing the present invention include the line of polycarbonate polyurethanes supplied under the trademark BIONATE® by The Polymer Technology Group Inc. (Berkeley, Calif.). For example, the very well-characterized grades of polycarbonate polyurethane polymer BIONATE® 80A, 55 and 90 are soluble in THF, DMF, DMAT, DMSO, or a mixture of two or more thereof, processable, reportedly have good mechanical properties, lack cytotoxicity, lack mutagenicity, lack carcinogenicity and are non-hemolytic. Another commercially-available elastomer suitable for use in practicing the present invention is the CHRONOFLEX® C line of biodurable medical grade polycarbonate aromatic polyurethane thermoplastic elastomers available from CardioTech International, Inc. (Woburn, Mass.).

Other possible embodiments of the materials used to fabricate the implants of this invention are described in co-pending, commonly assigned U.S. patent application Ser. No. 10/749,742, filed Dec. 30, 2003, titled “Reticulated Elastomeric Matrices, Their Manufacture and Use in Implantable Devices”, Ser. No. 10/848,624, filed May 17, 2004, titled “Reticulated Elastomeric Matrices, Their Manufacture and Use In Implantable Devices”, and Ser. No. 10/990,982, filed Jul. 27, 2004, titled “Endovascular Treatment Devices and Methods”, each of which is incorporated herein by reference in its entirely. NEEDS UPDATE FROM SENICH

The material for the implant or attachment or fixation member or retention member or device such as 40, 160, 174, 196 and 222 can be degradable or non-degradable materials or fiber-reinforced composites using degradable or non-degradable materials. The list of non-degradable materials for fixation member or retention member includes, but is not limited to, polymers such as polypropylene, polyethylene, polyethylene terepthalate (PET), Nylon 6, Nylon 6-6, polyimide, polyether imide, PEEK, or their mixtures and copolymers thereof. Additionally, the list of non-degradable materials for fixation member or retention member or attachment devices includes Teflon, fiber reinforced polymers, ceramics, etc., and metals such as, but not limited to, stainless steel, platinum, and nitinol. The list of degradable materials or degradable polymers for attachment device or fixation member or retention member include but not limited to, polymers such as polyglycolic acid (“PGA”), polylactic acid (“PLA”), poly D-lactide, Poly D-L lactide, polycaprolactic acid (“PCL”), poly-p-dioxanone (“PDO”), PGA/PLA copolymers, PGA/poly D-L Lacatide copolymers, PGA/PCL copolymers, PGA/PDO copolymers, PLA/PCL copolymers, PLA/PDO copolymers, PCL/PDO copolymers, or their mixtures and copolymers thereof, or combinations of any two or more of the foregoing.

The yield load, defined as force necessary for to start bending or deforming of the distal tips or extending point (such as 170, 190, 218 and 244) ranges from 5 Newtons (1.1 pound) to 70 Newtons (16 pounds) and preferably from 8 Newtons (1.8 pounds) to 50 Newtons (11.2 pounds). The break load is the maximum load for permanently deforming or breaking the anchor and ranges from 15 (3.4 pounds) Newtons to 250 Newtons (56.2 pounds) and preferably from 30 Newtons (6.7 pounds) to 100 Newtons (22.5 pounds). Although these ranges for yield loads and break loads are applicable to device or implant or fixation member or retention member made from polymer, preferably degradable polymers, they can apply to other materials of construction. However the yield loads and break loads are expected to be higher for metallic fixation member or retention member

It is within the scope of this invention that the elastomeric scaffold may optionally have a simple dip or spray polymer coating, the coating optionally comprising a pharmaceutically-active agent, such as a therapeutic agent or drug. In one embodiment the coating may be a solution and the polymer content in the coating solution is from about 1% to about 40% by weight. In another embodiment, the polymer content in the coating solution may be from about 1% to about 20% by weight. In another embodiment, the polymer content in the coating solution may be from about 1% to about 10% by weight.

In one embodiment of the invention, a biodurable reticulated elastomeric matrix has a coating comprising material selected to encourage cellular ingrowth and proliferation. The coating material can, for example, comprise a foamed coating of a biodegradable material, optionally, collagen, fibronectin, elastin, hyaluronic acid and mixtures thereof. Alternatively, the coating comprises a biodegradable polymer and an inorganic component.

In another embodiment, the reticulated biodurable elastomer is coated or impregnated with a material such as, for example, polyglycolic acid (“PGA”), polylactic acid (“PLA”), polycaprolactic acid (“PCL”), poly-p-dioxanone (“PDO”), PGA/PLA copolymers, PGA/PCL copolymers, PGA/PDO copolymers, PLA/PCL copolymers, PLA/PDO copolymers, PCL/PDO copolymers or combinations of any two or more of the foregoing.

The solvent or solvent blend for the coating solution is chosen with consideration given to, inter alia, the proper balancing the viscosity, deposition level of the polymer, wetting rate and evaporation rate of the solvent to properly coat solid phase as known to those in the art. In one embodiment, the solvent is chosen such the polymer is soluble in the solvent. In another embodiment, the solvent is substantially completely removed from the coating. In another embodiment, the solvent is non-toxic, non-carcinogenic and environmentally benign. Mixed solvent systems can be advantageous for controlling the viscosity and evaporation rates. In all cases, the solvent should not react with the coating polymer. Solvents include, but are not limited to, acetone, N-methylpyrrolidone (“NMP”), DMSO, toluene, methylene chloride, chloroform, 1,1,2-trichloroethane (“TCE”), various freons, dioxane, ethyl acetate, THF, DMF and DMAC.

In another embodiment, the film-forming coating polymer is a thermoplastic polymer that is melted, enters the pores of the elastomeric matrix and, upon cooling or solidifying, forms a coating on at least a portion of the solid material of the elastomeric matrix. In another embodiment, the processing temperature of the thermoplastic coating polymer in its melted form is above about 60° C. In another embodiment, the processing temperature of the thermoplastic coating polymer in its melted form is above about 90° C. In another embodiment, the processing temperature of the thermoplastic coating polymer in its melted form is above about 120° C.

In a further embodiment of the invention, described in more detail below, some or all of the pores of the elastomeric matrix are coated or filled with a cellular ingrowth promoter. In another embodiment, the promoter can be foamed. In another embodiment, the promoter can be present as a film. The promoter can be a biodegradable material to promote cellular invasion of the elastomeric matrix in vivo. Promoters include naturally occurring materials that can be enzymatically degraded in the human body or are hydrolytically unstable in the human body, such as fibrin, fibrinogen, collagen, elastin, hyaluronic acid and absorbable biocompatible polysaccharides, such as chitosan, starch, fatty acids (and esters thereof), glucoso-glycans and hyaluronic acid. In some embodiments, the pore surface of the elastomeric matrix is coated or impregnated, as described above, but substituting the promoter for the biocompatible polymer or adding the promoter to the biocompatible polymer, to encourage cellular ingrowth and proliferation.

In one embodiment, the coating or impregnating process is conducted so as to ensure that the product “composite elastomeric implantable device”, i.e., a reticulated elastomeric matrix and a coating, as used herein, retains sufficient resiliency after compression such that it can be delivery-device delivered, e.g., catheter, syringe or endoscope delivered. Some embodiments of such a composite elastomeric implantable device will now be described with reference to collagen, by way of non-limiting example, with the understanding that other materials may be employed in place of collagen, as described above.

Collagen may be infiltrated by forcing, e.g., with pressure, an aqueous collagen slurry, suspension or solution into the pores of an elastomeric matrix and lyophilized. The collagen may be Type I, II or III or mixtures thereof. In one embodiment, the collagen type comprises at least 90% collagen I. The concentration of collagen is from about 0.3% to about 2.0% by weight and the pH of the slurry, suspension or solution is adjusted to be from about 2.6 to about 5.0 at the time of lyophilization. Alternatively, collagen may be infiltrated by dipping an elastomeric matrix into a collagen slurry and lyophilized. The collagen can be cross-linked by cross-linking agent or addition of thermal energy under inert atmosphere or vacuum.

As compared with the uncoated reticulated elastomer, the composite elastomeric implantable device can have a void phase that is slightly reduced in volume. In one embodiment, the composite elastomeric implantable device retains good fluid permeability and sufficient porosity for ingrowth and proliferation of fibroblasts or other cells.

Optionally, the lyophilized collagen can be cross-linked to control the rate of in vivo enzymatic degradation of the collagen coating and to control the ability of the collagen coating to bond to the elastomeric matrix. Without being bound by any particular theory, it is thought that when the composite elastomeric implantable device is implanted, tissue-forming agents that have a high affinity to collagen, such as fibroblasts, will more readily invade the collagen-impregnated elastomeric matrix than the uncoated matrix. It is further thought, again without being bound by any particular theory, that as the collagen enzymatically degrades, new tissue invades and fills voids left by the degrading collagen while also infiltrating and filling other available spaces in the elastomeric matrix. Such a collagen coated or impregnated elastomeric matrix is thought, without being bound by any particular theory, to be additionally advantageous for the structural integrity provided by the reinforcing effect of the collagen within the pores of the elastomeric matrix which can impart greater rigidity and structural stability to various configurations of the elastomeric matrix.

The biodurable reticulated elastomeric matrix useful according to this invention can support cell types including cells secreting structural proteins and cells that produce proteins characterizing organ function. The ability of the elastomeric matrix to facilitate the co-existence of multiple cell types together and its ability to support protein secreting cells demonstrates the applicability of the elastomeric matrix in organ growth in vitro or in vivo and in organ reconstruction. In addition, the biodurable reticulated elastomeric matrix may also be used in the scale up of human cell lines for implantation to the body for many applications including implantation of fibroblasts, chondrocytes, osteoblasts, osteoclasts, osteocytes, synovial cells, bone marrow stromal cells, stem cells, fibrocartilage cells, endothelial cells, smooth muscle cells, adipocytes, cardiomyocytes, myocytes, keratinocytes, hepatocytes, leukocytes, macrophages, endocrine cells, genitourinary cells, lymphatic vessel cells, pancreatic islet cells, muscle cells, intestinal cells, kidney cells, blood vessel cells, thyroid cells, parathyroid cells, cells of the adrenal-hypothalamic pituitary axis, bile duct cells, ovarian or testicular cells, salivary secretory cells, renal cells, epithelial cells, nerve cells, stem cells, progenitor cells, myoblasts and intestinal cells.

New tissue can be obtained through implantation of cells seeded in elastomeric matrices (either prior to or concurrent to or subsequent to implantation). In this case, the elastomeric matrices may be configured either in a closed manner to protect the implanted cells from the body's immune system, or in an open manner so that the new cells can be incorporated into the body. Thus, in another embodiment, the cells may be incorporated, i.e., cultured and proliferated, onto the elastomeric matrix prior, concurrent or subsequent to implantation of the elastomeric matrix in the patient.

In one embodiment, the implantable device made from biodurable reticulated elastomeric matrix can be seeded with a type of cell and cultured before being inserted into the patient, optionally using a delivery-device, for the explicit purpose of tissue repair or tissue regeneration. It is necessary to perform the tissue or cell culture in a suitable culture medium with or without stimulus such as stress or orientation. The cells include fibroblasts, chondrocytes, osteoblasts, osteoclasts, osteocytes, synovial cells, bone marrow stromal cells, stem cells, fibrocartilage cells, endothelial cells and smooth muscle cells.

Surfaces on the biodurable reticulated elastomeric matrix possessing different pore morphology, size, shape and orientation may be cultured with different type of cells to develop cellular tissue engineering implantable devices that are specifically targeted towards orthopedic applications, especially in soft tissue attachment, repair, re-generation, augmentation and/or support encompassing spine, shoulder, knee, hand, joints, and in the growth of a prosthetic organ. In another embodiment, all the surfaces on the biodurable reticulated elastomeric matrix possessing similar pore morphology, size, shape and orientation may be so cultured.

In another embodiment, the film-forming polymer used to coat the reticulated elastomeric matrix can provide a vehicle for the delivery of and/or the controlled release of a pharmaceutically-active agent, for example, a drug, such as is described in the copending applications. In another embodiment, the pharmaceutically-active agent is admixed with, covalently bonded to and/or adsorbed in or on the coating of the elastomeric matrix to provide a pharmaceutical composition. In another embodiment, the components, polymers and/or blends used to form the foam comprise a pharmaceutically-active agent. To form these foams, the previously described components, polymers and/or blends are admixed with the pharmaceutically-active agent prior to forming the foam or the pharmaceutically-active agent is loaded into the foam after it is formed.

In one embodiment, the coating polymer and pharmaceutically-active agent have a common solvent. This can provide a coating that is a solution. In another embodiment, the pharmaceutically-active agent can be present as a solid dispersion in a solution of the coating polymer in a solvent.

A reticulated elastomeric matrix comprising a pharmaceutically-active agent may be formulated by mixing one or more pharmaceutically-active agent with the polymer used to make the foam, with the solvent or with the polymer-solvent mixture and foamed. Alternatively, a pharmaceutically-active agent can be coated onto the foam, in one embodiment, using a pharmaceutically-acceptable carrier. If melt-coating is employed, then, in another embodiment, the pharmaceutically-active agent withstands melt processing temperatures without substantial diminution of its efficacy.

Formulations comprising a pharmaceutically-active agent can be prepared by admixing, covalently bonding and/or adsorbing one or more pharmaceutically-active agents with the coating of the reticulated elastomeric matrix or by incorporating the pharmaceutically-active agent into additional hydrophobic or hydrophilic coatings. The pharmaceutically-active agent may be present as a liquid, a finely divided solid or another appropriate physical form. Typically, but optionally, the matrix can include one or more conventional additives, such as diluents, carriers, excipients, stabilizers and the like.

In another embodiment, a top coating can be applied to delay release of the pharmaceutically-active agent. In another embodiment, a top coating can be used as the matrix for the delivery of a second pharmaceutically-active agent. A layered coating, comprising respective layers of fast- and slow-hydrolyzing polymer, can be used to stage release of the pharmaceutically-active agent or to control release of different pharmaceutically-active agents placed in the different layers. Polymer blends may also be used to control the release rate of different pharmaceutically-active agents or to provide a desirable balance of coating characteristics (e.g., elasticity, toughness) and drug delivery characteristics (e.g., release profile). Polymers with differing solvent solubilities can be used to build-up different polymer layers that may be used to deliver different pharmaceutically-active agents or to control the release profile of a pharmaceutically-active agents.

The amount of pharmaceutically-active agent present depends upon the particular pharmaceutically-active agent employed and medical condition being treated. In one embodiment, the pharmaceutically-active agent is present in an effective amount. In another embodiment, the amount of pharmaceutically-active agent represents from about 0.01% to about 60% of the coating by weight. In another embodiment, the amount of pharmaceutically-active agent represents from about 0.01% to about 40% of the coating by weight. In another embodiment, the amount of pharmaceutically-active agent represents from about 0.1% to about 20% of the coating by weight.

Many different pharmaceutically-active agents can be used in conjunction with the reticulated elastomeric matrix. In general, pharmaceutically-active agents that may be administered via pharmaceutical compositions of this invention include, without limitation, any therapeutic or pharmaceutically-active agent (including but not limited to nucleic acids, proteins, lipids, and carbohydrates) that possesses desirable physiologic characteristics for application to the implant site or administration via a pharmaceutical compositions of the invention. Therapeutics include, without limitation, antiinfectives such as antibiotics and antiviral agents; chemotherapeutic agents (e.g., anticancer agents); anti-rejection agents; analgesics and analgesic combinations; anti-inflammatory agents; hormones such as steroids; growth factors (including but not limited to cytokines, chemokines, and interleukins) and other naturally derived or genetically engineered proteins, polysaccharides, glycoproteins and lipoproteins. These growth factors are described in The Cellular and Molecular Basis of Bone Formation and Repair by Vicki Rosen and R. Scott Thies, published by R. G. Landes Company, hereby incorporated herein by reference. Additional therapeutics include thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroids, non-steroidal anti-inflammatory agents, immunosuppressive agents, growth hormone antagonists, growth factors, dopamine agonists, radiotherapeutic agents, peptides, proteins, enzymes, extracellular matrix components, angiotensin-converting enzyme (ACE) inhibitors, free radical scavengers, chelators, antioxidants, anti polymerases, antiviral agents, photodynamic therapy agents and gene therapy agents.

Additionally, various proteins (including short chain peptides), growth agents, chemotatic agents, growth factor receptors or ceramic particles can be added to the foams during processing, adsorbed onto the surface or back-filled into the foams after the foams are made. For example, in one embodiment, the pores of the foam may be partially or completely filled with biocompatible resorbable synthetic polymers or biopolymers (such as collagen or elastin), biocompatible ceramic materials (such as hydroxyapatite), and combinations thereof, and may optionally contain materials that promote tissue growth through the device. Such tissue-growth materials include but are not limited to autograft, allograft or xenograft bone, bone marrow and morphogenic proteins. Biopolymers can also be used as conductive or chemotactic materials, or as delivery vehicles for growth factors. Examples include recombinant collagen, animal-derived collagen, elastin and hyaluronic acid. Pharmaceutically-active coatings or surface treatments could also be present on the surface of the materials. For example, bioactive peptide sequences (RGD's) could be attached to the surface to facilitate protein adsorption and subsequent cell tissue attachment. In a further embodiment of the invention, the pores of biodurable reticulated elastomeric matrix that are used to fabricate the implants of this invention are coated or filled with a cellular ingrowth promoter. In another embodiment, the promoter can be foamed. In another embodiment, the promoter can be present as a film. The promoter can be a biodegradable material to promote cellular invasion of pores biodurable reticulated elastomeric matrix that are used to fabricate the implants of this invention in vivo. Promoters include naturally occurring materials that can be enzymatically degraded in the human body or are hydrolytically unstable in the human body, such as fibrin, fibrinogen, collagen, elastin, hyaluronic acid and absorbable biocompatible polysaccharides, such as chitosan, starch, fatty acids (and esters thereof), glucoso-glycans and hyaluronic acid. In some embodiments, the pore surface of the biodurable reticulated elastomeric matrix that are used to fabricate the implants of this invention is coated or impregnated, as described in the previous section but substituting the promoter for the biocompatible polymer or adding the promoter to the biocompatible polymer, to encourage cellular ingrowth and proliferation.

Bioactive molecules include, without limitation, proteins, collagens (including types IV and XVIII), fibrillar collagens (including types I, II, III, V, XI), FACIT collagens (types IX, XII, XIV), other collagens (types VI, VII, XIII), short chain collagens (types VIII, X), elastin, entactin-1, fibrillin, fibronectin, fibrin, fibrinogen, fibroglycan, fibromodulin, fibulin, glypican, vitronectin, laminin, nidogen, matrilin, perlecan, heparin, heparan sulfate proteoglycans, decorin, filaggrin, keratin, syndecan, agrin, integrins, aggrecan, biglycan, bone sialoprotein, cartilage matrix protein, Cat-301 proteoglycan, CD44, cholinesterase, HB-GAM, hyaluronan, hyaluronan binding proteins, mucins, osteopontin, plasminogen, plasminogen activator inhibitors, restrictin, serglycin, tenascin, thrombospondin, tissue-type plasminogen activator, urokinase type plasminogen activator, versican, von Willebrand factor, dextran, arabinogalactan, chitosan, polyactide-glycolide, alginates, pullulan, gelatin and albumin.

Additional bioactive molecules include, without limitation, cell adhesion molecules and matricellular proteins, including those of the immunoglobulin (Ig; including monoclonal and polyclonal antibodies), cadherin, integrin, selectin, and H-CAM superfamilies. Examples include, without limitation, AMOG, CD2, CD4, CD8, C-CAM (CELL-CAM 105), cell surface galactosyltransferase, connexins, desmocollins, desmoglein, fasciclins, F11, GP Ib-IX complex, intercellular adhesion molecules, leukocyte common antigen protein tyrosine phosphate (LCA, CD45), LFA-1, LFA-3, mannose binding proteins (MBP), MTJC18, myelin associated glycoprotein (MAG), neural cell adhesion molecule (NCAM), neurofascin, neruoglian, neurotactin, netrin, PECAM-1, PH-20, semaphorin, TAG-1, VCAM-1, SPARC/osteonectin, CCN1 (CYR61), CCN2 (CTGF; Connective Tissue Growth Factor), CCN3 (NOV), CCN4 (WISP-1), CCN5 (WISP-2), CCN6 (WISP-3), occludin and claudin. Growth factors include, without limitation, BMP's (1-7), BMP-like Proteins (GFD-5, -7, -8), epidermal growth factor (EGF), erythropoietin (EPO), fibroblast growth factor (FGF), growth hormone (GH), growth hormone releasing factor (GHRF), granulocyte colony-stimulating factor (G-CSF), granulocyte-macrophage colony-stimulating factor (GM-CSF), insulin, insulin-like growth factors (IGF-I, IGF-II), insulin-like growth factor binding proteins (IGFBP), macrophage colony-stimulating factor (M-CSF), Multi-CSF (II-3), platelet-derived growth factor (PDGF), tumor growth factors (TGF-alpha, TGF-beta), tumor necrosis factor (TNF-alpha), vascular endothelial growth factors (VEGF's), angiopoietins, placenta growth factor (PIGF), interleukins, and receptor proteins or other molecules that are known to bind with the aforementioned factors. Short-chain peptides include, without limitation (designated by single letter amino acid code), RGD, EILDV, RGDS, RGES, RFDS, GRDGS, GRGS, GRGDTP and QPPRARI. One possible material for use in the present invention comprises a resiliently compressible composite polyurethane material comprising a hydrophilic foam coated on and throughout the pore surfaces of a hydrophobic foam scaffold. One suitable such material is the composite foam disclosed in co-pending, commonly assigned U.S. patent application Ser. No. 10/692,055, filed Oct. 22, 2003, Ser. No. 10/749,742, filed Dec. 30, 2003, Ser. No. 10/848,624, filed May 17, 2004, and Ser. No. 10/900,982, filed Jul. 27, 2004, each of which is incorporated herein by reference in its entirety. The hydrophobic foam provides support and resilient compressibility enabling the desired collapsing of the implant for delivery and reconstitution in situ.

The elastomeric matrix useful according to the invention may be molded into any of a wide variety of shapes and sizes during its formation or production. The shape may be a working configuration, such as any of the shapes and configurations described above, or the shape may be for bulk stock. Bulk stock items may subsequently be cut, trimmed, punched, milled, or otherwise shaped for end use. The sizing and shaping can be carried out by, for example, using a blade, a rotating knife, a serrated blade, a computer aided CNCV machine, a punch, a drill or a laser. In each of these embodiments, the processing temperature or temperatures of the cutting tools for shaping and sizing can be ambient temperature or an elevated temperature, e.g., greater than about 100° C. In another embodiment, the processing temperature(s) of the cutting tools for shaping and sizing can be greater than about 130° C. In one embodiment, the biodurable reticulated elastomeric matrix can be frozen and cut or shaped cryogenically. Finishing steps can include, in one embodiment, trimming of macrostructural surface protrusions, such as struts or the like, which can irritate biological tissues. In another embodiment, finishing steps can include heat annealing. Annealing can be carried out before or after final cutting and shaping. Annealing will be carried out temperatures in excess of 100° C. for from about 1 to about 6 hours.

Biodurable reticulated elastomeric matrices, or an implantable device system comprising such matrices, can be sterilized by any method known to the art including gamma irradiation, autoclaving, ethylene oxide sterilization, infrared irradiation and electron beam irradiation. In one embodiment, biodurable elastomers used to fabricate the elastomeric matrix tolerate such sterilization without loss of useful physical and mechanical properties. The use of gamma irradiation can potentially provide additional cross-linking to enhance the performance of the device.

In one embodiment, the sterilized products may be packaged in uncompressed state in sterile packages of paper, polymer or other suitable material. In embodiment, the elastomeric matrix remains uncompressed in such a package for typical commercial storage and distribution times, which will commonly exceed 3 months and may be up to 1 or 5 years from manufacture to use. In another embodiment, within such packages, the elastomeric matrix is compressed within a retaining member to facilitate its loading into a delivery-device, such as a catheter or endoscope, in a compressed configuration. In another embodiment, the elastomeric matrix comprises an elastomer with a compression set enabling it to expand to a substantial proportion of its pre-compressed volume, e.g., at 25° C., to at least 50% of its pre-compressed volume. In another embodiment, expansion occurs after the elastomeric matrix remains compressed in such a package for typical commercial storage and distribution times, which will commonly exceed 3 months and may be up to 1 or 5 years from manufacture to use. If desired, the reticulated elastomeric implants or implants can be rendered radiopaque to allow for visualization of the implants in situ by the clinician during and after the procedure, employing radioimaging. Any suitable radiopaque agent that can be covalently bound, adhered or otherwise attached to the reticulated polymeric implants may be employed including without limitation, tantalum, titanium and barium sulfate or other suitable material known to those skilled in the art. In addition to incorporating radiopaque agents such as tantalum into the implant material itself, a further embodiment of the invention encompasses the use of radiopaque metallic components to impart radiopacity to the implant. For example, thin filaments comprised of metals with or without shape memory properties such as platinum or nitinol can be embedded into the implant and may be in the form of a straight or curved wire, helical or coil-like structure, umbrella structure, or other structure generally known to those skilled in the art. Alternatively, a metallic frame around the implant may also be used to impart radiopacity. The metallic frame may be in the form of a tubular structure, a helical or coil-like structure, an umbrella structure, or other structure generally known to those skilled in the art. In one embodiment, the metallic implants incorporated in or surrounding the orthopedic or spinal implant for gripping or attachment or positioning or fastening of the implant at the target site can be used to impart radiopacity. Attachment of radiopaque metallic components to the implant can be accomplished by means including but not limited to chemical bonding or adhesion, suturing, pressure fitting, compression fitting, and other physical methods.

According to the invention the reticulated elastomeric matrix can be appropriately shaped to form a closure device to seal the access opening in the annulus resulting from a discetomy to reinforce and stabilize the disc annulus in case of herniated disc, also known as disc prolapse or a slipped or bulging disc. The implantable device is compressed and delivered into the annulus opening by a trocar, cannula, or catheter with assisted visualization through an endoscopic instrument such as a laproscope, arthroscope, or cystoscope, preferably the cannula used during the discectomy procedure. In another embodiment, the implantable device is not compressed and delivered into the annulus opening by a trocar, cannula, or catheter with assisted visualization through an endoscopic instrument such as a laproscope, arthroscope, or cystoscope, preferably the cannula used during the discectomy procedure. The device can be secured into the opening by at least the following two mechanisms: first, the outwardly resilient nature of the reticulated solid phase can provide a mechanical means for preventing migration; and, second, the reticulated solid phase can serve as a scaffold to support fibrocartilage growth into the interconnected void phase of the elastomeric matrix. Additional securing may be obtained by the use of anchors, sutures or biological glues and adhesives, as known to those in the art. The closure device can support fibrocartilage ingrowth into the elastomeric matrix of the implantable device. Once released at the site, the reticulated elastomeric matrix expands resiliently to about its original, relaxed size and shape subject, of course, to its compression set limitation and any desired flexing, draping or other conformation to the site anatomy that the implantable device may adopt.

In one embodiment, cellular entities such as fibroblasts and tissues can invade and grow into the reticulated elastomeric matrix. In due course, such ingrowth can extend into the interior pores and interstices of the inserted reticulated elastomeric matrix. Eventually, the elastomeric matrix can become substantially filled with proliferating cellular ingrowth that provides a mass that can occupy the site or the void spaces in it. The types of tissue ingrowth possible include, but are not limited to, fibrous tissues and endothelial tissues.

In another embodiment, the implantable device or device system causes cellular ingrowth and proliferation throughout the site, throughout the site boundary, or through some of the exposed surfaces, thereby sealing the site. Over time, this induced fibrous or fibrovascular entity resulting from tissue ingrowth can cause the implantable device to be incorporated into the conduit. Tissue ingrowth can lead to very effective resistance to migration of the implantable device over time. It may also prevent recanalization of the conduit. In another embodiment, over the course of time, for example, for 2 weeks to 3 months to 1 year, the implanted reticulated elastomeric matrix becomes completely filled and/or encapsulated by tissue, fibrous tissue, scar tissue or the like.

The properties of the reticulated elastomeric matrix can be engineered to match the application by, e.g., controlling the amount of cross-linking, amount of crystallinity, chemical composition, chemical type of the solvent or solvent blend (when a solvent is used in processing), annealing conditions, curing conditions, and degree of reticulation. Unlike biodegradable polymers, when used as a scaffold, the reticulated elastomeric matrix maintains its physical characteristics and performance in vivo over long periods of time. Thus, it does not initiate undesirable tissue response as is observed for biodegradable implants when they break down and degrade. The high void content and degree of reticulation of the reticulated elastomeric matrix allows tissue ingrowth and proliferation of cells within the matrix. In one embodiment, the ingrown tissue and/or proliferated cells occupy from about 51% to about 99% of the volume of interconnected void phase of the original implantable device, thereby providing functionality, such as load bearing capability, of the original tissue that is being repaired or replaced.

### EXAMPLES

### Example 1

### Fabrication of a Cross-Linked Reticulated Polyurethane Matrix

Aromatic isocyanates, RUBINATE 9258 (from Huntsman; comprising a mixture of 4,4′-MDI and 2,4′-MDI), were used as the isocyanate component. RUBINATE 9258 contains about 68% by weight 4,4′-MDI, about 32% by weight 2,4′-MDI and has an isocyanate functionality of about 2.33 and is a liquid at 25° C. A polyol-1,6-hexamethylene carbonate (PC 1733, Stahl Chemicals) i.e., a diol, with a molecular weight of about 1,000 Daltons, was used as the polyol component and is a solid at 25° C. Glycerol was the chain extender, and water was used as the blowing agent. The blowing catalyst were tertiary amine 33% triethylenediamine in dipropylene glycol (DABCO 33LV supplied by Air Products) and Niax-A1 (supplied by Air Products). A silicone-based surfactant was used (TEGOSTAB® BF 2370, supplied by Goldschmidt). The cell-opener was ORTEGOL® 501 (supplied by Goldschmidt). A viscosity depressant (Propylene carbonate supplied by Sigma-Aldrich) was also used. The proportions of the components that were used is given in the following table:

silicone-based surfactant was used (TEGOSTAB® BF 2370, supplied by Goldschmidt). The cell-opener was ORTEGOL® 501 (supplied by Goldschmidt). A viscosity depressant (Propylene carbonate supplied by Sigma-Aldrich) was also used. The proportions of the components that were used is given in the following table:

| Ingredient | Parts by Weight |
|---|---|
| Polyol Component - PC 1733, Stahl | 100 |
| Chemicals | |
| Glycerine | 4.92 |
| Viscosity Depressant - Propylene | 11.6 |
| carbonate | |
| Surfactant - TEGOSTAB ® BF 2370 | 4.40 |
| Cell Opener - ORTEGOL ® 501 | 4.0 |
| Isocyanate Component RUBINATE | 99.78 |
| 9258 | |
| Isocyanate Index | 1.00 |
| Distilled Water | 3.36 |
| Blowing Catalyst Dabco 33 LV | 1.0 |
| Blowing Catalyst Niax-A1 | 0.06 |

The polyol was liquefied at 70° C. in an air circulation oven, and was weighed into a polyethylene cup. Viscosity depressant (propylene carbonate) was added to the polyol and mixed with a drill mixer equipped with a mixing shaft at 3100 rpm for 15 seconds (mix-1). Surfactant (Tegostab BF-2370) was added to mix-1 and mixed for additional 15 seconds (mix-2). Cell opener (Ortogel 501) was added to mix-2 and mixed for 15 seconds (mix-3). Isocyanate (Rubinate 9258) was added to mix-3 and mixed for 60±10 seconds (system A).

Distilled water was mixed with both blowing catalyst (Dabco 33LV and Niax A1) and glycerine in a small plastic cup by using a tiny glass rod for 60 seconds (System B).

System B was poured into System A as quickly as possible without spilling and with vigorous mixing with a drill mixer for 10 seconds and poured into cardboard box of 9 in.×8 in.×5 in., which is covered inside with aluminum foil. The foaming profile was as follows: mixing time of 10 sec., cream time of 18 sec. and rise time of 75 sec.

Two minutes after beginning of foam mixing, the foam was placed in the oven at 100-105° C. for curing for 65 minutes. The foam is taken from the oven and cooled for 15 minutes at room temperature. The skin was cut with the band saw, and the foam was pressed by hand from all sides to open the cell windows. The foam was put back into an air-circulation oven for post-curing at 100°-105° C. for an additional 5 hours.

The average pore diameter of the foam, as observed by optical microscopy, as shown in the micrographs of FIGS. 15 and 16, was between 150 and 300 μm.

The subsequent foam testing was carried out in accordance with ASTM D3574. Density was measured with specimens measuring 50 mm×50 mm×25 mm. The density was calculated by dividing the weight of the sample by the volume of the specimen; a value of 2.75 lbs/ft³ was obtained.

Tensile tests were conducted on samples that were cut both parallel and perpendicular to the direction of foam rise. The dog-bone shaped tensile specimens were cut from blocks of foam each about 12.5 mm thick, about 25.4 mm wide and about 140 mm long. Tensile properties (strength and elongation at break) were measured using an INSTRON Universal Testing Instrument Model 1122 with a cross-head speed of 500 mm/min (19.6 inches/minute). The average tensile strength, measured from two orthogonal directions parallel and perpendicular with respect to foam rise, were 67.6 psi and 56.44 psi, respectively. The elongation to break was approximately 46%.

In the subsequent reticulation procedure, a block of foam was placed into a pressure chamber, the doors of the chamber were closed and an airtight seal was maintained. The pressure was reduced to remove substantially all of the air in the foam. A combustible ratio of hydrogen to oxygen gas was charged into the chamber for enough time to permeate all the samples. The gas in the chamber was then ignited by a spark plug. The ignition exploded the gasses within the foam cell structure. This explosion blew out many of the foam cell windows, thereby creating a reticulated elastomeric matrix structure.

### Example 2

### Fabrication of a Cross-Linked Reticulated Polyurethane Matrix

Aromatic isocyanates, RUBINATE 9258 (from Huntsman; comprising a mixture of 4,4′-MDI and 2,4′-MDI), were used as the isocyanate component. RUBINATE 9258 contains about 68% by weight 4,4′-MDI, about 32% by weight 2,4′-MDI and has an isocyanate functionality of about 2.33 and is a liquid at 25° C. A polyol-1,6-hexamethylene carbonate (Desmophen LS 2391, Bayer Polymers), i.e., a diol, with a molecular weight of about 2,000 Daltons, was used as the polyol component and is a solid at 25° C. Water was used as the blowing agent. The blowing catalyst was the tertiary amine 33% triethylenediamine in dipropylene glycol (DABCO 33LV supplied by Air Products). A silicone-based surfactant was used (TEGOSTAB® BF 2370, supplied by Goldschmidt). The cell-opener was ORTEGOL® 501 (supplied by Goldschmidt). A viscosity depressant (Propylene carbonate supplied by Sigma-Aldrich) was also used. The proportions of the components that were used is given the following table:

TABLE 2

| Ingredient | Parts by Weight |
|---|---|
| Polyol Component - Desmophen LS | 100 |
| 2391 | |
| Viscosity Depressant - Propylene | 5.76 |
| carbonate | |
| Surfactant - TEGOSTAB ® BF 2370 | 2.16 |
| Cell Opener - ORTEGOL ® 501 | 0.48 |
| Isocyanate Component RUBINATE | 53.8 |
| 9258 | |
| Isocyanate Index | 1.00 |
| Distilled Water | 2.82 |
| Blowing Catalyst | 0.44 |

The polyol Desmophen LS 2391 was liquefied at 70° C. in an air circulation oven, and 150 gms of it was weighed into a polyethylene cup. 8.7 g of viscosity depressant (propylene carbonate) was added to the polyol and mixed with a drill mixer equipped with a mixing shaft at 3100 rpm for 15 seconds (mix-1). 3.3 g of surfactant (Tegostab BF-2370) was added to mix-1 and mixed for additional 15 seconds (mix-2). 0.75 g of cell opener (Ortogel 501) was added to mix-2 and mixed for 15 seconds (mix-3). 80.9 g of isocyanate (Rubinate 9258) is added to mix-3 and mixed for 60±10 seconds (System A).

4.2 g of distilled water was mixed with 0.66 g of blowing catalyst (Dabco 33LV) in a small plastic cup by using a tiny glass rod for 60 seconds (System B).

System B was poured into System A as quickly as possible without spilling and with vigorous mixing with a drill mixer for 10 seconds and poured into cardboard box of 9 in.×8 in.×5 in., which was covered inside with aluminum foil. The foaming profile was as follows: mixing time of 10 sec., cream time of 18 sec. and rise time of 85 sec.

Two minutes after beginning of foam mixing, the foam was placed in the oven at 100-105° C. for curing for 60 minutes. The foam was taken from the oven and cooled for 15 minutes at room temperature. The skin was cut with the band saw, and the foam was pressed by hand from all sides to open the cell windows. The foam was put back in an air-circulation oven for postcuring at 100°-105° C. for additional 5 hours.

The average pore diameter of the foam, as observed by optical microscopy, as shown in FIGS. 17 and 18, was between 150 and 450 μm.

Subsequent foam testing was carried out in accordance with ASTM D3574. Density was measured with specimens measuring 50 mm×50 mm×25 mm. The density was calculated by dividing the weight of the sample by the volume of the specimen; a value of 2.5 lbs/ft³ was obtained.

Tensile tests were conducted on samples that were cut both parallel and perpendicular to the direction of foam rise. The dog-bone shaped tensile specimens were cut from blocks of foam each about 12.5 mm thick, about 25.4 mm wide and about 140 mm long. Tensile properties (strength and elongation at break) were measured using an INSTRON Universal Testing Instrument Model 1122 with a cross-head speed of 500 nun/min (19.6 inches/minute). The average tensile strength, measured from two orthogonal directions with respect to foam rise, was 24.64±2.35 psi. The elongation to break was approximately 215±12%.

Compressive strengths of the foam were measured with specimens measuring 50 mm×50 mm×25 mm. The tests were conducted using an INSTRON Universal Testing Instrument Model 1122 with a cross-head speed of 10 mm/min (0.4 inches/min). The compressive strength at 50% was about 12±3 psi. The compression set after subjecting the sample to 50% compression for 22 hours at 40° C. and releasing the stress was 2%.

Tear resistance strength of the foam was measured with specimens measuring approximately 152 mm×25 mm×12.7 mm. A 40 mm cut was made on one side of each specimen. The tear strength was measured using an INSTRON Universal Testing Instrument Model 1122 with a cross-head speed of 500 mm/min (19.6 inches/minute). The tear strength was determined to be about 2.9±0.1 lbs/inch.

The pore structure and its inter-connectivity is measured by Liquid Extrusion Porosimeter (manufactured by Porous Materials, Inc. (Ithaca, N.Y.). In this test, the pores of a 25.4 mm diameter sample is filled with a wetting fluid having a surface tension of 19 dynes/cm and loaded in a sample chamber with a 27 micron diameter pore membrane being placed under the sample. The pressure of air in the chamber space above the wetted sample is increased slowly so that the liquid is extruded from the pores of the sample. For low surface tension fluid, the contact angle is taken to be zero and the wetting liquid that spontaneously fills the pore of the test sample also spontaneously fill the pores of the membranes when the former is emptied under pressure with larger pores emptying out at lower pressures and smaller pores emptying out at higher pressure. The displaced liquid passes through the membrane and its volume measured. The differential pressure p required to displace liquid from a pore is related to its diameter D, surface tension of the liquid γ and the contact angle θ by the relation p=4γ cos θ/D. The gas pressure gives the pore diameter and the volume of the displaced liquid gives the pore volume or the intrusion volume accessible to the low surface tension liquid. Again measurement of liquid flow (water in this case) without the membrane under the sample and using similar pressure-flow methods yields liquid permeability. The liquid intrusion volume for the foam is 4 cc/gm and permeability of water through the foam is 1 lit/min/psi/sq cm.

In the subsequent reticulation procedure, a block of foam was placed into a pressure chamber, the doors of the chamber are closed, and an airtight seal was maintained. The pressure is reduced to remove substantially all of the air in the foam. A combustible ratio of hydrogen to oxygen gas was charged into the chamber for enough time to permeate all the samples. The gas in the chamber was then ignited by a spark plug. The ignition explodes the gasses within the foam cell structure. This explosion blew out many of the foam cell windows, thereby creating a reticulated elastomeric matrix structure.

Tensile tests were conducted on reticulated samples that were cut both parallel and perpendicular to the direction of foam rise. The dog-bone shaped tensile specimens were cut from blocks of foam each about 12.5 mm thick, about 25.4 mm wide and about 140 mm long. Tensile properties (strength and elongation at break) were measured using an INSTRON Universal Testing Instrument Model 1122 with a cross-head speed of 500 mm/min (19.6 inches/minute). The average tensile strength, measured from two orthogonal directions with respect to foam rise, was 23.5 psi. The elongation to break was approximately 194%.

Post reticulation compressive strengths of the foam were measured with specimens measuring 50 mm×50 mm×25 mm. The tests were conducted using an INSTRON Universal Testing Instrument Model 1122 with a cross-head speed of 10 mm/min (0.4 inches/min). The compressive strength at 50% was about 6.5 psi.

The pore structure and its inter-connectivity is measured by Liquid Extrusion Porosimeter. The liquid intrusion volume for the reticulated foam is 28 cc/gm and permeability of water through the foam is 413 lit/min/psi/sq cm. The results demonstrate the interconnected and continuous pore structure of the reticulated foam compared to the un-reticulated foam.

### Example 3

### Fabrication of a Crosslinked Polyurethane Matrix

The aromatic isocyanate RUBINATE 9258 (from Huntsman) was used as the isocyanate component. RUBINATE 9258, which is a liquid at 25° C., contains 4,4′-MDI and 2,4′-MDI and has an isocyanate functionality of about 2.33. A diol, poly(1,6-hexanecarbonate)diol (POLY-CD CD220 from Arch Chemicals) with a molecular weight of about 2,000 Daltons was used as the polyol component and was a solid at 25° C. Distilled water was used as the blowing agent. The blowing catalyst used was the tertiary amine triethylenediamine (33% in dipropylene glycol; DABCO 33LV from Air Products). A silicone-based surfactant was used (TEGOSTAB® BF 2370 from Goldschmidt). A cell-opener was used (ORTEGOL® 501 from Goldschmidt). The viscosity modifier propylene carbonate (from Sigma-Aldrich) was present to reduce the viscosity. The proportions of the components that were used are set forth in the following table:

TABLE 3

| Ingredient | Parts by Weight |
|---|---|
| Polyol Component | 100 |
| Viscosity Modifier | 5.80 |
| Surfactant | 0.66 |
| Cell Opener | 1.00 |
| Isocyanate Component | 47.25 |
| Isocyanate Index | 1.00 |
| Distilled Water | 2.38 |
| Blowing Catalyst | 0.53 |

The polyol component was liquefied at 70° C. in a circulating-air oven, and 100 g thereof was weighed out into a polyethylene cup. 5.8 g of viscosity modifier was added to the polyol component to reduce the viscosity, and the ingredients were mixed at 3100 rpm for 15 seconds with the mixing shaft of a drill mixer to form “Mix-1”. 0.66 g of surfactant was added to Mix-1, and the ingredients were mixed as described above for 15 seconds to form “Mix-2”. Thereafter, 1.00 g of cell opener was added to Mix-2, and the ingredients were mixed as described above for 15 seconds to form “Mix-3”. 47.25 g of isocyanate component were added to Mix-3, and the ingredients were mixed for 60±10 seconds to form “System A”.

2.38 g of distilled water was mixed with 0.53 g of blowing catalyst in a small plastic cup for 60 seconds with a glass rod to form “System B”.

System B was poured into System A as quickly as possible while avoiding spillage. The ingredients were mixed vigorously with the drill mixer as described above for 10 seconds and then poured into a 22.9 cm×20.3 cm×12.7 cm (9 in.×8 in.×5 in.) cardboard box with its inside surfaces covered by aluminum foil. The foaming profile was as follows: 10 seconds mixing time, 17 seconds cream time, and 85 seconds rise time.

Two minutes after the beginning of foaming, i.e., the time when Systems A and B were combined, the foam was placed into a circulating-air oven maintained at 100-105° C. for curing for from about 55 to about 60 minutes. Then, the foam was removed from the oven and cooled for 15 minutes at about 25° C. The skin was removed from each side using a band saw. Thereafter, hand pressure was applied to each side of the foam to open the cell windows. The foam was replaced into the circulating-air oven and postcured at 100-105° C. for an additional four hours.

The average pore diameter of the foam, as determined from optical microscopy observations, was greater than about 275 μm.

The following foam testing was carried out according to ASTM D3574: Bulk density was measured using specimens of dimensions 50 mm×50 mm×25 mm. The density was calculated by dividing the weight of the sample by the volume of the specimen. A density value of 2.81 lbs/ft³ (0.0450 g/cc) was obtained.

Tensile tests were conducted on samples that were cut either parallel to or perpendicular to the direction of foam rise. The dog-bone shaped tensile specimens were cut from blocks of foam. Each test specimen measured about 12.5 mm thick, about 25.4 mm wide, and about 140 mm long; the gage length of each specimen was 35 mm and the gage width of each specimen was 6.5 mm. Tensile properties (tensile strength and elongation at break) were measured using an INSTRON Universal Testing Instrument Model 1122 with a cross-head speed of 500 mm/min (19.6 inches/minute). The average tensile strength perpendicular to the direction of foam rise was determined as 29.3 psi (20,630 kg/m²). The elongation to break perpendicular to the direction of foam rise was determined to be 266%.

The measurement of the liquid flow through the material is measured in the following way using a iquid permeability apparatus or Liquid Permeaeter (Porous Materials, Inc., Ithaca, N.Y.). The foam sample was 8.5 mm in thickness and covered a hole 6.6 mm in diameter in the center of a metal plate that was placed at the bottom of the Liquid Permeaeter filled with water. Thereafter, the air pressure above the sample was increased slowly to extrude the liquid from the sample and the permeability of water through the foam was determined to be 0.11 L/min/psi/cm².

### Example 4

### Reticulation of a Cross-Linked Polyurethane Foam

Reticulation of the foam described in Example 3 was carried out by the following procedure: A block of foam measuring approximately 15.25 cm×15.25 cm×7.6 cm (6 in.×6 in.×3 in.) was placed into a pressure chamber, the doors of the chamber were closed, and an airtight seal to the surrounding atmosphere was maintained. The pressure within the chamber was reduced to below about 100 millitorr by evacuation for at least about two minutes to remove substantially all of the air in the foam. A mixture of hydrogen and oxygen gas, present at a ratio sufficient to support combustion, was charged into the chamber over a period of at least about three minutes. The gas in the chamber was then ignited by a spark plug. The ignition exploded the gas mixture within the foam. The explosion was believed to have at least partially removed many of the cell walls between adjoining pores, thereby forming a reticulated elastomeric matrix structure.

The average pore diameter of the reticulated elastomeric matrix, as determined from optical microscopy observations, was greater than about 275 μm. A scanning electron micrograph image of the reticulated elastomeric matrix of this example (not shown here) demonstrated, e.g., the communication and interconnectivity of pores therein.

The density of the reticulated foam was determined as described above in Example 3. A post-reticulation density value of 2.83 lbs/ft³ (0.0453 g/cc) was obtained.

Tensile tests were conducted on reticulated foam samples as described above in Example 3. The average post-reticulation tensile strength perpendicular to the direction of foam rise was determined as about 26.4 psi (18,560 kg/m²). The post-reticulation elongation to break perpendicular to the direction of foam rise was determined to be about 250%. The average post-reticulation tensile strength parallel to the direction of foam rise was determined as about 43.3 psi (30,470 kg/m²). The post-reticulation elongation to break parallel to the direction of foam rise was determined to be about 270%.

Compressive tests were conducted using specimens measuring 50 mm×50 mm×25 mm. The tests were conducted using an INSTRON Universal Testing Instrument Model 1122 with a cross-head speed of 10 mm/min (0.4 inches/minute). The post-reticulation compressive strengths at 50% compression, parallel to and perpendicular to the direction of foam rise, were determined to be 1.53 psi (1,080 kg/m²) and 0.95 psi (669 kg/m²), respectively. The post-reticulation compressive strengths at 75% compression, parallel to and perpendicular to the direction of foam rise, were determined to be 3.53 psi (2,485 kg/m²) and 2.02 psi (1,420 kg/m²), respectively. The post-reticulation compression set, determined after subjecting the reticulated sample to 50% compression for 22 hours at 25° C. then releasing the compressive stress, parallel to the direction of foam rise, was determined to be about 4.5%.

The resilient recovery of the reticulated foam was measured by subjecting 1 inch (25.4 mm) diameter and 0.75 inch (19 mm) long foam cylinders to 75% uniaxial compression in their length direction for 10 or 30 minutes and measuring the time required for recovery to 90% (“t-90%”) and 95% (“t-95%”) of their initial length. The percentage recovery of the initial length after 10 minutes (“r-10”) was also determined. Separate samples were cut and tested with their length direction parallel to and perpendicular to the foam rise direction. The results obtained from an average of two tests are shown in the following table:

TABLE 4

| Time | | | | |
|---|---|---|---|---|
| compressed | Test Sample | t-90% | t-95% | r-10 |
| (min) | Orientation | (sec) | (sec) | (%) |
| 10 | Parallel | 6 | 11 | 100 |
| 10 | Perpendicular | 6 | 23 | 100 |
| 30 | Parallel | 9 | 36 | 99 |
| 30 | Perpendicular | 11 | 52 | 99 |

In contrast, a comparable foam with little to no reticulation typically has t-90 values of greater than about 60-90 seconds after 10 minutes of compression.

The measurement of the liquid flow through the material was measured in the following way using a Liquid permeability apparatus or Liquid Permeaeter (Porous Materials, Inc., Ithaca, N.Y.). The foam samples were between 7.0 and 7.7 mm in thickness and covered a hole 8.2 mm in diameter in the center of a metal plate that was placed at the bottom of the Liquid Permeaeter filled with water. The water was allowed to extrude through the sample under gravity and the permeability of water through the foam was determined to be 180 L/min/psi/cm² in the direction of foam rise and 160 L/min/psi/cm² in the perpendicular to foam rise.

### Example 5

### Fabrication of a Cross-Linked Reticulated Polyurethane Matrix

A cross-linked Polyurethane Matrix was made using similar starting materials and following procedures similar to the one described in Example 3. Glycerol was used as an additional starting material. The proportions of the components that were used are set forth in the following table:

TABLE 5

| Ingredient | Parts by Weight |
|---|---|
| PolyCD ™ CD220(g) | 100 |
| Propylene carbonate (g) | 5.80 |
| Tegostab BF-2370 (g) | 1.50 |
| Ortegol 501 (g) | 1.00 |
| Rubinate 9258 (g) | 49.29 |
| Distiled water) (g) | 1.80 |
| Dabco 33 LV (g) | 0.50 |
| Glycerine (g) | 2.46 |

The reaction profile is as follows:

| | |
|---|---|
| Mixing time | 10 |
| Cream time | 27 |
| Rise time | 120 |

The average pore diameter of the foam, as determined from optical microscopy observations, was greater than about 225 μm.

The following foam testing was carried out according to ASTM D3574: Bulk density was measured using specimens of dimensions 50 mm×50 mm×25 mm. The density was calculated by dividing the weight of the sample by the volume of the specimen. A density value of 3.65 lbs/ft³ (0.060 g/cc) was obtained.

Tensile tests were conducted on samples that were cut perpendicular to the direction of foam rise. The dog-bone shaped tensile specimens were cut from blocks of foam. Each test specimen measured about 12.5 mm thick, about 25.4 mm wide, and about 140 mm long; the gage length of each specimen was 35 mm and the gage width of each specimen was 6.5 mm. Tensile properties (tensile strength and elongation at break) were measured using an INSTRON Universal Testing Instrument Model 1122 with a cross-head speed of 500 mm/min (19.6 inches/minute). The average tensile strength perpendicular to the direction of foam rise was determined as 37.8 psi (26,500 kg/m²). The elongation to break perpendicular to the direction of foam rise was determined to be 141%.

Reticulation of the foam described above was carried out by the following procedure: A block of foam measuring approximately 15.25 cm×15.25 cm×7.6 cm (6 in.×6 in.×3 in.) was placed into a pressure chamber, the doors of the chamber were closed, and an airtight seal to the surrounding atmosphere was maintained. The pressure within the chamber was reduced to below about 100 millitorr by evacuation for at least about two minutes to remove substantially all of the air in the foam. A mixture of hydrogen and oxygen gas, present at a ratio sufficient to support combustion, was charged into the chamber over a period of at least about three minutes. The gas in the chamber was then ignited by a spark plug. The ignition exploded the gas mixture within the foam. The explosion was believed to have at least partially removed many of the cell walls between adjoining pores, thereby forming a reticulated elastomeric matrix structure.

A scanning electron micrograph image of the reticulated elastomeric matrix of this example (not shown here) demonstrated, e.g., the communication and interconnectivity of pores therein.

The density of the reticulated foam was determined as described above and a value of 4.00 lbs/ft³ (0.0656 g/cc) was obtained.

Tensile tests were conducted on reticulated foam samples as described above and the average post-reticulation tensile strength perpendicular to the direction of foam rise was determined as about 35.3 psi (24,680 kg/m²). The post-reticulation elongation to break perpendicular to the direction of foam rise was determined to be about 125%.

Compressive tests were conducted using specimens measuring 50 mm×50 mm×25 mm. The tests were conducted using an INSTRON Universal Testing Instrument Model 1122 with a cross-head speed of 10 mm/min (0.4 inches/minute). The post-reticulation compressive strengths perpendicular to the direction of foam rise at 50% and 75% compression strains were determined to be 3.83 psi (2,680 kg/m²) and 9.33 psi (6,530 kg/m²), respectively.

### Example 6

### Testing in a Rabbit Model

An example of a device according to the invention, a cylindrical scaffold of reticulated polycarbonate prepared consistent with Examples 3 to 5, referred to as the “ARDX implant”, was used for annular repair in the rabbit model of degenerative disc disease. This model is considered a standard model to evaluate the vertebral disc. See, for example, H. S. An et al., “Biological Repair of Intervertebral Disc,” Spine, 2003 Aug. 1; 28 (15 Suppl.); D. G. Anderson et al., “Comparative Gene Expression Profiling of Normal and degenerative Discs: Analysis of a Rabbit Annular Laceration Mode,” Spine. 2002 Jun. 15; 27(12): 1291-96; and M. W. Kroeber et al., “New in Vivo Animal Model to Create Intervertebral Disc Degeneration and to Investigate the Effects of Therapeutic Strategy to Stimulate Disc Regeneration,” Spine, 2002 Dec. 1; 27(23): 2684-90. Four adult female New Zealand rabbits were utilized for the experiment. Under a general anesthetic via a posterior-lateral approach, the lumbar spine was exposed. The annulus of disc spaces from L1 to L5 were then incised in with a #15 scalpel laterally to induce the traumatic injury. Three of the annular defects were repaired with the ARDX implant, which was positioned into the spinal annular defect and secured with a non-resorbable suture. The fourth disc space was left un-repaired as a control. The animals were sacrificed at four weeks, and the spinal segments were processed for histology with H&E and SO stains. The findings at harvest showed excellent tolerance of the implants and grossly maintained disc space. The histology showed the preservation of the disc space and intact nucleus.

The ARDX implant was well integrated with good tissue in-growth, as is shown in the micrograph (No2L45 SO stain 100x) of FIG. 19 and the close-up view in FIG. 20, where the implant 130 abuts nucleus 132 adjacent to annulus 134. Annulus 134 is in turn adjacent to vertebral end plate 136. In the detail shown in FIG. 20 new tissue growth 138 can be seen. A strut or projection 140 from implant 130 can be seen. The early regeneration of matrix secretion and organized collagen fibers preserved the disc space and prevented degeneration when compared to control samples.

Overall the ARDX implant device promoted repair and regeneration of spinal annulus and disc in the rabbit model.

### Example 7

### Tissue Response of Implant Placed in Animal Spine

A mushroom shaped device with the mushroom head diameter being 12 mm and mushroom stem or body diameter being 8 mm was machined from reticulated elastomeric matrix having similar properties as those in Example 2 and prepared consistent with Example 2 but with the composition set forth in the following table:

TABLE 6

| Ingredients | Parts by Weight |
|---|---|
| Desmophen LS 2391 | 100 |
| Propilene carbonate | 5.86 |
| Distilled water | 2.83 |
| BF-2370 | 2.44 |
| Dabco 33 LV | 0.49 |
| Ortegol 501 | 0.52 |
| Rubinate9258 | 53.45 |
| Isocyanate index | 100 |

The young-adult Yucatan Mini-Swine was used for this experiment. Four 3-mm antero-lateral annulotomies were performed in each animal, and about 500 mg of nucleus pulposus were removed from each animal. The devices were implanted at the inter-vertebral discs (IVD), within the annular defect at three disc levels in the spinal column, and the devices were covered with a titanium mesh to prevent extrusion.

All animals survived with no complications until the study endpoint of 6 weeks, after which they were sacrificed and subjected to a limited necropsy consisting of an examination of the implantation sites or the implanted annulus fibrosus-intervertebral. Whole discs (L1-L2, L2-L3, L3-L4, L4-L5) were dissected free, and specimens corresponding to the implant or control area were isolated for processing.

As can be seen in the micrographs of FIGS. 72 and 73, a substantial amount of vascularized granulation tissue ingrowth (moderate) was seen in the harvested samples. However, a fibrous or well-demarcated fibrovascular encapsulation of the implant devices was not observed. The inflammatory response to the bare device matrix (DM) was minimal and in line with expected response to a foreign body placed in the IVD. The low-grade inflammation (I) was characterized by rare neutrophils, minimal number of macrophages, and minimal to mild number of giant cells. The material appeared to be intact and unaffected by the inflammatory cells. The device material's surface was smooth, and the giant cells had no material present in their cytoplasm. No degradation products were noted in any of the tissues. The device material (DM) was surrounded by neutrophils (n) and red blood cells (r) near the center of the intervertebral space. A mild inflammatory response was observed away from the center. There was no evidence of fibrous capsule formation.

The device demonstrated favorable response for annular repair following discectomy in an animal model paralleling human clinical usage. The implant was well integrated with good tissue in-growth

### Example 8

### Placement of Device with Associated Anchor in a Surgically-Created Annulus in a Sheep

A reticulated cross-linked biodurable elastomeric polycarbonate polyurethane urea-urethane matrix was made by the following procedure:

The aromatic isocyanate Mondur MRS-20 (from Bayer Corporation) was used as the isocyanate component. Mondur MRS-20 is a liquid at 25° C. Mondur MRS-20 contains 4,4′-diphenylmethane diisocyanate (MDI) and 2,4′-MDI and has an isocyanate functionality of about 2.2. A diol, poly(1,6-hexanecarbonate) diol (POLY-CD220 from Arch Chemicals) with a molecular weight of about 2,000 Daltons, was used as the polyol component and was a solid at 25° C. Distilled water was used as the blowing agent. The catalysts used were the amines triethylene diamine (33% by weight in dipropylene glycol); DABCO 33LV (from Air Products) and bis(2-dimethylaminoethyl)ether (23% by weight in dipropylene glycol); NIAX® A-133 (from GE Silicones). Silicone-based surfactants TEGOSTAB® BF 2370 and TEGOSTAB® B-8305 (from Goldschmidt) were used for cell stabilization. A cell-opener was used (ORTEGOL® 501 from Goldschmidt). The viscosity modifier propylene carbonate (from Sigma-Aldrich) was present to reduce the viscosity. Glycerine (99.7% USP Grade) and 1,4-butanediol (99.75% by weight purity, from Lyondell) were added to the mixture as, respectively, a cross-linking agent and a chain extender. The proportions of the ingredients that were used is given in the table below.

TABLE 7

| Ingredient | Parts by Weight |
|---|---|
| Polyol Component | 100 |
| Isocyanate Component | 52.96 |
| Isocyanate Index | 1.00 |
| Viscosity Modifier | 5.80 |
| Cell Opener | 2.00 |
| Distilled Water | 1.95 |
| B-8305 Surfactant | 0.70 |
| BF 2370 Surfactant | 0.70 |
| 33LV Catalyst | 0.45 |
| A-133 Catalyst | 0.12 |
| Glycerine | 2.00 |
| 1,4-Butanediol | 0.80 |

The isocyanate index, a quantity well known in the art, is the mole ratio of the number of isocyanate groups in a formulation available for reaction to the number of groups in the formulation that are able to react with those isocyanate groups, e.g., the reactive groups of diol(s), polyol component(s), chain extender(s), water and the like, when present. The isocyanate component of the formulation was placed into the component A metering system of an Edge Sweets Bench Top model urethane mixing apparatus and maintained at a temperature of about 20-25° C.

The polyol was liquefied at about 70° C. in an oven and combined with the viscosity modifier and cell opener in the aforementioned proportions to make a homogeneous mixture. This mixture was placed into the component B metering system of the Edge Sweets apparatus. This polyol component was maintained in the component B system at a temperature of about 65-70° C.

The remaining ingredients from Table 7 were mixed in the aforementioned proportions into a single homogeneous batch and placed into the component C metering system of the Edge Sweets apparatus. This component was maintained at a temperature of about 20-25° C. During foam formation, the ratio of the flow rates, in grams per minute, from the supplies for component A: component B: component C was about 8:16:1.

The above components were combined in a continuous manner in the 250 cc mixing chamber of the Edge Sweets apparatus that was fitted with a 10 mm diameter nozzle placed below the mixing chamber. Mixing was promoted by a high-shear pin-style mixer operating in the mixing chamber. The mixed components exited the nozzle into a rectangular cross-section release-paper coated mold. Thereafter, the foam rose to substantially fill the mold. The resulting mixture began creaming about 10 seconds after contacting the mold and was at full rise within 120 seconds. The top of the resulting foam was trimmed off and the foam was placed into a 100° C. curing oven for 5 hours.

Following curing, the sides and bottom of the foam block were trimmed off, and then the foam was placed into a reticulator device comprising a pressure chamber, the interior of which was isolated from the surrounding atmosphere. The pressure in the chamber was reduced to remove substantially all the air in the cured foam. A mixture of hydrogen and oxygen gas, present at a ratio sufficient to support combustion, was charged into the chamber. The pressure in the chamber was maintained above atmospheric pressure for a sufficient time to ensure gas penetration into the foam. The gas in the chamber was then ignited by a spark plug and the ignition exploded the gas mixture within the foam. To minimize contact with any combustion products and to cool the foam, the resulting combustion gases were removed from the chamber and replaced with about 25° C. nitrogen immediately after the explosion. Then, the above-described reticulation process was repeated one more time. Without being bound by any particular theory, the explosions were believed to have at least partially removed many of the cell walls or “windows” between adjoining cells in the foam, thereby creating open pores and leading to a reticulated elastomeric matrix structure.

The average cell diameter or other largest transverse dimension of the reticulated elastomeric matrix, as determined from optical microscopy observations, was about 525 μm. Scanning electron micrograph (SEM) images of the reticulated elastomeric matrix of this example demonstrated, e.g., the network of cells interconnected via the open pores therein. The average pore diameter or other largest transverse dimension of the reticulated elastomeric matrix, as determined from SEM observations, was about 205 μm.

The following tests were carried out on the thus-formed reticulated elastomeric matrix, obtained from reticulating the foam, using test methods based on ASTM Standard D3574. Bulk density was measured using reticulated elastomeric matrix specimens of dimensions 5.0 cm×5.0 cm×2.5 cm. The post-reticulation density was calculated by dividing the weight of the specimen by the volume of the specimen. A density value of 3.29 lbs/ft³ (0.053 g/cc) was obtained.

Tensile tests were conducted on reticulated elastomeric matrix specimens that were cut either parallel to or perpendicular to the foam-rise direction. The dog-bone shaped tensile specimens were cut from blocks of reticulated elastomeric matrix. Each test specimen measured about 1.25 cm thick, about 2.54 cm wide, and about 14 cm long. The gage length of each specimen was 3.5 cm and the gage width of each specimen was 6.5 mm. Tensile properties (tensile strength and elongation at break) were measured using an INSTRON Universal Testing Instrument Model 3342 with a cross-head speed of 50 cm/min (19.6 inches/minute). The average post-reticulation tensile strength perpendicular to the foam-rise direction was determined to be about 34.3 psi (24,115 kg/m²). The post-reticulation elongation to break perpendicular to the foam-rise direction was determined to be about 124%. The average post-reticulation tensile strength parallel to the foam-rise direction was determined to be about 61.4 psi (43,170 kg/m²). The post-reticulation elongation to break parallel to the foam-rise direction was determined to be about 122%.

Compressive tests were conducted using reticulated elastomeric matrix specimens measuring 5.0 cm×5.0 cm×2.5 cm. The tests were conducted using an INSTRON Universal Testing Instrument Model 1122 with a cross-head speed of 1 cm/min (0.4 inches/minute). The post-reticulation compressive strength at 50% compression, parallel to the foam-rise direction, was determined to be about 2.1 psi (1,475 kg/m²). The post-reticulation compression set, determined after subjecting the reticulated specimen to 50% compression for 22 hours at 25° C. then releasing the compressive stress, parallel to the foam-rise direction, was determined to be about 8.5%.

The resilient recovery of the reticulated elastomeric matrix was measured by subjecting rectangular parallelepiped specimens, each 1 inch (2.54 cm) high (in the foam-rise direction)×1.25 inches×1.25 inches (3.18 cm×3.18 cm), to a 50% uniaxial compression in the foam-rise direction and then, while maintaining that uniaxial compression, imparting a dynamic loading of ±5% strain at a frequency of 1 Hz for 5000 cycles or 100,000 cycles, also in the foam-rise direction. The time required for recovery to 67% (“t-67%”) and 90% (“t-90%”) of the specimens' initial height of 1 inch (2.54 cm) was measured and recorded. The results obtained are shown in the following table:

TABLE 8

| | Test Specimen | | |
|---|---|---|---|
| No. of Cycles at | Orientation | | |
| 50% | Relative to | | |
| Compression ± 5% | Foam-Rise | t-67% | t-90% |
| Strain at 1 Hz | Direction | (sec) | (sec) |
| 5,000 | Parallel | 0.7 | 46 |
| 100,000 | Parallel | 84 | 2370 |

Liquid permeability through the reticulated elastomeric matrix was measured in the foam-rise direction using a Model 101 A Automated Liquid Permeaeter liquid permeability apparatus (from Porous Materials, Inc., Ithaca, N.Y.). The cylindrical reticulated elastomeric matrix specimens tested were between 7.0-7.7 mm in diameter and 13-14 mm in length. A flat end of a specimen was placed in the center of a metal plate that was placed at the bottom of the Liquid Permeaeter apparatus. To measure liquid permeability, water was allowed to extrude under pressure from the specimen's end through the specimen along its axis. The permeability of water through the reticulated elastomeric matrix was determined to be 321 Darcy in the foam-rise direction.

Device shaped as in FIGS. 17 and 18 were machined from the matrix with the following dimensions: a proximal cylindrical section or portion: 0-5 mm; Height—10 mm, an expanded middle cylindrical section (Mushroom portion): ø—7 mm; Height—3 mm and distal cylindrical section (Top hat) portion: ø—5 mm; Height—4 mm.

A fixation element or retention member for fixation shaped as FIG. 20 (Arrowhead Gusset design) was injection molded from a biodegradable 95/5 (L-lactide/glycolide) copolymer with an inherent viscosity of 2.38. Molding was done using a 55 ton Arburg Injection Molder with a screw diameter of 15 mm and with a hopper that was outfitted with a nitrogen purge to keep the resin dry. The temperature profile in the molder from the feed zone to the compression zone was between 40° C. and 199° C. while the nozzle and mold temperatures were 199° C. and 40° C., respectively. The injection pressure was set to 25,000 psi (17.6×10⁶ kg/m²) and the hold pressure was set to 10,000 psi (7.0×10⁶ kg/m²). A maximum injection speed of 0.27 cu·in/second (4.4 cc/sec), a cooling time of 30 seconds, and an overall cycle time of 40 seconds were used for the molding process.

A fixation element or retention member for fixation was attached to the device by securing the top hat region of the fixation element (nose part) to the biodurable reticulated matrix from which the device is made. A braided polyester fiber similar in diameter to a size 4-0 suture was used.

Two female sheep weighing between 55 kg and 58 Kg were used for this experiment. A left retroperitoneal approach was used. Four consecutive lumbar discs were exposed. A 2.5 mm×5 mm cruciate cut was made on anterior-lateral annulus fibrosus and a partial discectomy was performed. Devices containing biodegradable fixation element were successfully inserted into the annular defect sites using an WD rongeur (KM 47-730): 2 mm×6 mm. The devices were very stable after implantation, as noted by pulling the suture that is attached to the fixation element.

After 4 weeks of implantation, the study sheep were euthanized according to the research facility's standard procedure and a complete post-mortem was performed. The five major organs (heart, liver, spleen, lungs and kidneys) and local lymph nodes were grossly normal. The lumbar spines were removed en bloc. The surrounding soft tissues were grossly dissected and the discs examined. The device in the defects created in the discs did not extrude and were flush with the external annulus wall, as shown in FIG. 74.

There was a peripheral tissue attachment (ingrowth) to the devices. The outside layer of ingrowth of tissue or tissue attachment held the device in place. The histology slides revealed that there was very dense fibrous tissue ingrowth into the voids of the matrix material as shown in the micrograph of FIG. 75.

### Example 9

### Test of the Migration Characteristics of Annular Closure Devices (ACDs) Following Implantation and Fatigue Testing in Cadaveric Intervertebral Discs

A reticulated cross-linked biodurable elastomeric polycarbonate urea-urethane matrix was made by the procedure described in Example 8, with the exception that the ingredients used and their proportions are given in the following table:

TABLE 9

| Ingredient | Parts by Weight |
|---|---|
| Polyol Component | 100 |
| Isocyanate Component | 52.37 |
| Isocyanate Index | 1.00 |
| Viscosity Modifier | 5.80 |
| Cell Opener | 2.00 |
| Distilled Water | 2.15 |
| B-8305 Surfactant | 0.70 |
| BF 2370 Surfactant | 0.72 |
| 33LV Catalyst | 0.55 |
| Glycerine | 2.00 |
| 1,4-Butanediol | 1.95 |

The average cell diameter or other largest transverse dimension of the reticulated elastomeric matrix, as determined from optical microscopy observations, was about 576 μm. Scanning electron micrograph (SEM) images of the reticulated elastomeric matrix of this example demonstrated, e.g., the network of cells interconnected via the open pores therein. The average pore diameter or other largest transverse dimension of the reticulated elastomeric matrix, as determined from SEM observations, was about 281 μm.

The following tests were carried out on the thus-formed reticulated elastomeric matrix, obtained from reticulating the foam, using test methods based on ASTM Standard D3574. Bulk density was measured using reticulated elastomeric matrix specimens of dimensions 5.0 cm×5.0 cm×2.5 cm. The post-reticulation density was calculated by dividing the weight of the specimen by the volume of the specimen. A density value of 3.23 lbs/ft³ (0.053 g/cc) was obtained.

Tensile tests were conducted on reticulated elastomeric matrix specimens that were cut either parallel to or perpendicular to the foam-rise direction. The dog-bone shaped tensile specimens were cut from blocks of reticulated elastomeric matrix. Each test specimen measured about 1.25 cm thick, about 2.54 cm wide, and about 14 cm long. The gage length of each specimen was 3.5 cm and the gage width of each specimen was 6.5 mm. Tensile properties (tensile strength and elongation at break) were measured using an INSTRON Universal Testing Instrument Model 3342 with a cross-head speed of 50 cm/min (19.6 inches/minute). The average post-reticulation tensile strength perpendicular to the foam-rise direction was determined to be about 40 psi (28,120 kg/m²). The post-reticulation elongation to break perpendicular to the foam-rise direction was determined to be about 135%. The average post-reticulation tensile strength parallel to the foam-rise direction was determined to be about 55 psi (38,665 kg/m²). The post-reticulation elongation to break parallel to the foam-rise direction was determined to be about 126%.

Compressive tests were conducted using reticulated elastomeric matrix specimens measuring 5.0 cm×5.0 cm×2.5 cm. The tests were conducted using an INSTRON Universal Testing Instrument Model 1122 with a cross-head speed of 1 cm/min (0.4 inches/minute). The post-reticulation compressive strength at 50% compression, parallel to the foam-rise direction, was determined to be about 2.0 psi (1,406 kg/m²). The post-reticulation compression set, determined after subjecting the reticulated specimen to 50% compression for 22 hours at 25° C. then releasing the compressive stress, parallel to the foam-rise direction, was determined to be about 7.5%.

The resilient recovery of the reticulated elastomeric matrix was measured by subjecting rectangular parallelepiped specimens, each 1 inch (2.54 cm) in height (in the foam-rise direction) and a cross-section of 1.25 inches×1.25 inches (3.18 cm×3.18 cm), to a 50% uniaxial compression in the foam-rise direction and then, while maintaining that uniaxial compression, imparting a dynamic loading of ±5% strain at a frequency of 1 Hz for 5000 cycles or 100,000 cycles, also in the foam-rise direction. The time required for recovery to 67% (“t-67%”) and 90% (“t-90%”) of the specimens' initial height of 1 inch (2.54 cm) was measured and recorded. The results obtained are shown in the following table:

TABLE 10

| | Test Specimen | | |
|---|---|---|---|
| No. of Cycles at | Orientation | | |
| 50% | Relative to | | |
| Compression ± 5% | Foam-Rise | t-67% | t-90% |
| Strain at 1 Hz | Direction | (sec) | (sec) |
| 5,000 | Parallel | — | 123 |
| 100,000 | Parallel | 50 | 3845 |

Liquid permeability through the reticulated elastomeric matrix was measured in the foam-rise direction using a Model 101 A Automated Liquid Permeaeter liquid permeability apparatus (from Porous Materials, Inc., Ithaca, N.Y.). The cylindrical reticulated elastomeric matrix specimens tested were between 7.0-7.7 mm in diameter and 13-14 mm in length. A flat end of a specimen was placed in the center of a metal plate that was placed at the bottom of the Liquid Permeaeter apparatus. To measure liquid permeability, water was allowed to extrude under pressure from the specimen's end through the specimen along its axis. The permeability of water through the reticulated elastomeric matrix was determined to be 215 Darcy in the foam-rise direction.

Devices shaped as in FIGS. 17 and 18 were machined from the matrix with the following dimensions: a proximal cylindrical section or portion: ø—5 mm; Height—10 mm, an expanded middle cylindrical section (Mushroom portion): ø—7 mm; Height—3 mm and distal cylindrical section (Top hat) portion: ø—5 mm; Height—4 mm.

A fixation element or retention member for fixation shaped as FIG. 20 (Arrowhead Gusset design) was injection molded from a biodegradable 95/5 (L-lactide/glycolide) copolymer with an inherent viscosity of 2.38. Molding was done using a 55 ton Arburg Injection Molder with a screw diameter of 15 mm and with a hopper that was outfitted with a nitrogen purge to keep the resin dry. The temperature profile in the molder from the feed zone to the compression zone was between 40 C and 199° C. while the nozzle and mold temperatures were 199° C. and 40° C., respectively. The injection pressure was set to 25,000 psi (17.6×10⁶ kg/m²) and the hold pressure was set to 10,000 psi (7.0×10⁶ kg/m²). A maximum injection speed of 0.27 cu·in/second (4.4 cc/sec), a cooling time of 30 seconds and an overall cycle time of 40 seconds were used for the molding process.

A fixation element or retention member for fixation was attached to the device by securing the top hat region of the fixation element (nose part) to the biodurable reticulated matrix from which the device is made. A braided polyester fiber similar in diameter to a size 4-0 suture was used.

Segments from human spinal specimens were utilized for this study. In all cases, the specimens were imaged with X-rays and screened for gross anatomical defects. Intact spine specimens were maintained in a freezer at −20° C. until approximately 24 hours prior to testing. Specimens were thawed to room temperature and all residual musculature was removed via careful dissection. Throughout preparation and testing, the specimens were kept moist with a wrapping of saline-soaked gauze.

Two-level spinal lumbar or lumbosacral FSUs were harvested. Care was taken to preserve all ligamentous attachments and maintain segmental integrity. For each FSU, the cephalad and caudad vertebrae were rigidly embedded in a urethane potting compound using Kirschner wires and metal screws as needed. The segments were potted so that the mid-plane of the intervertebral disc visually appeared horizontal. Care was taken to ensure that the central axis of the potted construct was located at the intersection of the mid sagittal plane and a plane parallel with the mid-frontal plane that was located posteriorly two-thirds of the A/P disc width. Sufficient space was left to access the disc for discectomy and to insert the annulus closure device.

A standard posterior laminectomy was performed, with a slightly larger exposure to permit insertion of a custom measuring device. Then a cruciate-cut annulotomy was performed along with a partial nuclectomy. The volume of nucleus removed was determined using a saline displacement method. The volume of nuclear material removed from the various specimens ranged between 0.3 ml and 1.0 ml. Both devices with its associated anchor and devices without any attached anchor were inserted unilaterally for testing.

All FSUs were tested using a standard stiffness protocol with custom constrained fixtures in an INSTRON 8521S servohydraulic load frame (Instron Corp. Canton Mass.). The potting surrounding the lower vertebra was rigidly held to an X-Y table while the potting surrounding the upper vertebra was contacted with a spherical bearing. Each FSU was tested in one of two mechanical test modes (compression with flexion or compression with contralateral lateral bending). In all cases, two sets of loading protocols were followed: 40 N to 400 N compression for 5,000 cycles and then 120 N to 1200 N compression for 5,000 cycles. The specimens were aligned so that the sagittal plane included one of the adjustment axes for the X-Y table. In this way, the load could be offset a prescribed amount. The point of load application was 6.25 mm from the central axis of the FSU so that the theoretical applied moment was approximately 2.5 Nm and 7.5 Nm, respectively. Each FSU was cycled at a constant frequency of 1 Hz. Loads and moments in three orthogonal directions were acquired at 50 Hz utilizing a six-axis load cell (AMTI, Watertown, Mass.) and Instron 8500+ electronics. At 1000 cycle intervals, a custom measurement gauge was used to assess migration of the ACD and a video camera was used to record the angulation of the FSU. The test was to be halted if the ACD completely protruded from the annulus. Testing was performed in air, with the FSU wrapped in saline-soaked gauze. The number of cycles, amount of FSU angulation, and device migration were recorded. The various test conditions regarding dynamic compression testing data of devices with and without anchors following implantation and fatigue testing in cadaveric intervertrebal discs are provided in the table below:

TABLE 11

| | | | | | Change in | |
|---|---|---|---|---|---|---|
| | | | | | endplate angle | |
| | | | | | (°) | |
| Device | Specimen | | Nuclectomy | Test | 2.5 Nm | 7.5 Nm |
| Type | Gender/Age | Level | Volume (mL) | Mode | moment | moment |
| Device | F/71 | L1/L2 | 0.4 | RLB | 1 | 1 |
| Device | F/71 | L3/L4 | 0.3 | FLE | 0 | 0 |
| Device | F/71 | L5/S1 | 1.0 | RLB | 0 | 0 |
| Device + Anchor | F/34 | L1/L2 | 0.9 | RLB | 1 | 1 |
| Device + Anchor | F/34 | L3/L4 | 0.5 | RLB | 1 | 1 |
| Device + Anchor | M/73 | L2/L3 | 0.5 | FLE | 0 | 0 |

L = lumbar;
S = sacral,
RLB = right lateral bending,
FLE = flexion

Devices without attached anchor (or anchorless device) were successfully inserted. At the end of the test, the custom measuring device generally showed little (less than 2 mm) relative movement between the annular wall and the device. After testing, these devices generally were either flush or slightly inset (less than 1 mm) to the annular wall,

Devices incorporating an anchor (or anchored device) were successfully inserted. At the end of the test, no specimens exhibited obvious signs (cracking sound, sudden expulsion of marrow, etc.) of compression fractures during testing. The custom measuring devices generally showed little (less than 1.5 mm) relative movement between the annular wall and the device. After testing, these devices generally were either flush or inset (less than 0.5 mm to 2.0 mm) to the annular wall.

In both cases of device alone (anchorless device) and device incorporating anchor (anchored device), no substantial migration (either extrusion away from the nucleus or intrusion towards the nucleus) of annular closure devices (ACDs) was observed following implantation and fatigue testing in cadaveric intervertebral discs.

### Example 10

### Prevention of Extrusion from Simulated Annulotomy for Device with and Without Anchors

A reticulated cross-linked biodurable elastomeric polycarbonate polyurethane urea matrix was made following procedures similar to the one described in Example 9.

Devices shaped similar to FIGS. 15 and 16 with a proximal cylindrical section and a distal cylindrical mushroom section with the proximal cylindrical section (body portion) having a diameter of 8 mm and the a distal cylindrical mushroom section (or the mushroom head) having diameters varying from 8 mm to 12 mm were machined from the reticulated elastomeric matrix. There were no slits in the device. The height of the mushroom head varied from 4 to 8 mm. The fixation element or retention members for fixation and shaped as arrows were injection molded from a commercial grade polycarbonate containing 10% short glass fibers (Grade RTP 301 made by RTP company). The fixation element was shaped as FIG. 19 (Arrowhead Base Design) had a 1.25 mm diameter longitudinal base member (or stem) making a 45° angle with both the 1 mm diameter angularly extending arms. The fixation element or the retention member was attached or incorporated into the mushroom shaped device by a braided polyester fiber similar in diameter to a size 4-0 suture.

The experiment was intended to understand the failure mode and extrusion pressure of different configurations of the device by an in vitro test method. The developed method measured both the hydrostatic pressure at failure and the distance that the device extruded from a 5 mm diameter circular defect as the hydrostatic pressure, similar to that experienced in a human spine, was applied. The equipment that the test method utilized was a dual piston chamber with water in the upper piston and a layer of PVC plastisol in the lower piston. As the upper piston was compressed with an external screw, the increasing hydrostatic pressure was transferred to the PVC plastisol in the lower piston. Hydrostatic pressure measurements were measured from the water in the upper piston with a standard fluid pressure gauge. A silicone window measuring 12 mm×10 mm, (with a 5 mm diameter round defect) and having hardness of A65 was attached to the lower piston and the hole would allow for extrusion of the PVC plastisol layer through this defect with the application of pressure. Plugging the hole with the device would prevent extrusion but the potential for the latter would increase as the pressure on the system increased.

Different device configurations, with and without the attached fixation element or retention members for fixation were inserted into the defect and the displacement of the devices measured as the pressure was increased in the system. Devices were wetted in water for 5 minutes before inserting them for testing. The device was deemed to be satisfactorily meeting the requirements if it did not extrude at 2.5 MPa (362 psi) extrusion pressure. Pressure to extrude 2 mm of the device was noted; the device retracted back if the extrusion was below 2 mm. Blow out pressure is the pressure at which the layer of PVC plastisol extruded out of the hole in the silicone window and could be measure only up to 600 psi, which was the maximum that the pressure gage could record. The table below shows that devices with various mushroom head dimensions, and both with or without incorporating fixation element or retention members for fixation, can withstand a pressure of 2.5 MPa and prevent extrusion of the device and the layer of PVC plastisol behind it. Also, it takes fairly high pressure to extrude 2 mm of the device and the device distinctly shows that it can regain its original shape even after achieving 2 m extrusion.

TABLE 12

| Mushroom | Mushroom | Anchor | | Anchor | |
|---|---|---|---|---|---|
| Head | Head | used or | Presure to | failed at | Blow Out |
| Diameter | Thck. | not | extrude 2 mm | 2.5 MPa | Pressure |
| mm | mm | Yes/No | psi | Yes/No | psi (MPa) |
| 10 | 4 | YS | 340 | NO | 600/4.14 |
| 10 | 4 | YS | 450 | NO | 600/4.14 |
| 10 | 8 | NO | 300 | NO | 600/4.14 |
| 10 | 8 | NO | 300 | NO | 600/4.14 |
| 8 | 5 | YS | 380 | NO | 400/2.76 |
| 8 | 5 | YS | 280 | NO | 600/4.14 |
| 10 | 8 | NO | 300 | NO | 600/4.14 |
| 10 | 8 | YS | 280 | NO | 600/4.14 |
| 12 | 4 | NO | 330 | NO | 390/2.69 |
| 12 | 4 | NO | 310 | NO | 600/4.14 |

### Example 11

### Testing of Biodegradable Anchors

Fixation elements or retention members for fixation shaped as FIG. 21 (Arrowhead Web design) were injection molded from a biodegradable 95/5 (L-lactide/glycrolide) copolymer with an inherent viscosity of 2.38 following the process described in Example 8. Similar anchors were also made from poly L-lactide polymer with an inherent viscosity of 2.42 and following similar injection molding conditions.

The anchors are tested for their in vitro degradation properties. The anchors are placed in bottles containing a phosphate buffer solution at a ph of 7.3. The bottles containing the anchors are placed in a water bath maintained at a temperature of 37° C. The anchors are taken out and tested after 10 days and 23 days and tested for mechanical integrity by using a push-out test.

A push-out test method was developed to determine the load necessary to push the anchors through a 5 mm diameter hole in order to simulate resistance of the anchor in the spine. The test was performed to gain an understanding of the load that the anchor can withstand before being pushed out of the annulotomy hole. The test was performed on an INSTRON Universal Testing Instrument Model 3342 with a cross-head speed of 12.5 cm/min (0.5 inches/minute. Special fixtures were designed to hold the anchors in the two jaws. The top of the anchor was mounted in a fixture in the movable upper jaw of the machine. The movable jaw was then lowered so that the stem of the anchor passes through the hole (5 mm diameter) in the lower fixture and the wing tips of the anchor (at the bottom of the side legs) just touch the flat surface of the lower fixture. The lower fixture was mounted in the fixed jaw of the INSTRON testing machine. The fixtures were made of Aluminum. Once the anchor was mounted in the jaws, the Anchor Push out Test was performed at the above-mentioned speed. The software plotted the load versus displacement curve and the yield load (that is where the tip of the anchors start to bend) and the flattening load (that is where the tip of the anchors are collapsed) were evaluated and recoded. The maximum load that the anchor could resist as the fixtures holding the anchors approached each other before overloading the load cell limit of and extension can be obtained from the plot.

The table below shows the test results for dry anchors and anchors tested after 10 and 24 days in the in vitro bath. The results indicate that the yield load and extension increases as the time in the in vitro bath increases whereas there is a decrease in the flattening load. It was observed that none of the anchors broke during the test.

TABLE 13

| | In vitro | Max. | Yield | Flattening Load for |
|---|---|---|---|---|
| | time | Force | Load | Anchor legs |
| Anchor | Days | N | N | N |
| PLA Web | 0 | 90.05 | 10.56 | 52.23 |
| PLA Web | 10 | 90.13 | 18.13 | 44.39 |
| PLA Web | 24 | 90.09 | 18.46 | 51.73 |
| 95/5 Web | 0 | 90.08 | 16.95 | 47.34 |
| 95/5 Web | 10 | 90.09 | 21.22 | 43.54 |
| 95/5 Web | 24 | 90.10 | 26.06 | 57.12 |

Both type of anchors were able to maintain their mechanical integrity with no significant loss in property in 24 days.

### Example 12

### Placement of Device with Associated Fixation Element in a Surgically-created Annulus in a Sheep

An experiment was conducted in a similar way to that presented in Example 8. The reticulated cross-linked biodurable elastomeric polycarbonate urea-urethane matrix was made by the same procedure as in Example 8.

Devices shaped in as FIGS. 15 and 16 and containing slits were machined from the matrix with the following dimensions: a proximal cylindrical section or portion: ø—5 mm; Height—10 mm, an expanded middle cylindrical section (Mushroom portion): ø—7 mm; Height—3 mm.

A fixation element or retention member for fixation shaped as FIG. 21 (Arrowhead Web design) was injection molded from a biodegradable 95/5 (L-lactide/glycolide) copolymer with an inherent viscosity of 2.38 using the same method described in Example 8.

A fixation element or retention member for fixation was attached to the device by securing the top hat region of the fixation element (nose part) to the biodurable reticulated matrix from which the device is made by using methods similar to Example 8.

Devices containing biodegradable fixation element were successfully inserted into the annular defect sites in sheep following methods similar to those presented in Example 8. The devices were very stable after implantation, as noted by pulling the suture that is attached to the fixation element.

### Example 13

### Pullout Testing of Gusseted and Web B2 Biodegradable Fixation Element

A fixation element or retention member for fixation shaped as FIG. 20 (Arrowhead Gusset design) and FIG. 21 (Arrowhead Web design) were injection molded from a biodegradable 95/5 (L-lactide/glycolide) copolymer with an inherent viscosity of 2.38 following the process described in Example 8.

A Pullout test method was performed to determine the load necessary to pull the fixation elements or retention member for fixations from the inner annular wall of the sheep cadaver lumbar spine using methods similar to those described in Example 8. The test was performed on an INSTRON Model 3342 with Load Cell of 100N; a crosshead speed of 100 mm/min (4.0 inches/minute), and the distance between the movable jaw or grip and the fixation element is 45 mm. A test fixture and cable ties were used to secure the sheep cadaver lumbar spine in place.

A standard annulotomy and disectomy on sheep lumbar discs were performed through anterior-lateral approach. The defect size was about 5-6 mm in width and 2.5 mm in height. Fixation elements with 2-0 braided polyester suture (DEKNATEL, Code# 113-D) was loaded into the inserter and the fixation elements were inserted into the disc cavities. The fixation elements were pulled back to firmly engaging them with the inner annular wall. The spine was secured to the test fixture and the fixation element was positioned in the annular site and approximately aligned to the center of the grip. The suture was pulled slightly and attached to the upper grip or jaw such that the suture was vertically straight and perpendicular to the table surface without any excess tension. The movable upper jaw or grip was closed over the suture. Once the suture was mounted in the jaws, the Pull Out Test was performed at the above-mentioned speed and at least 5 repeat runs were done for each of the two fixation element design (Arrowhead Gusset design and Arrowhead Web design). The software plotted the load versus displacement curve and average maximum load for failure of the fixation element. This pullout test data of fixation elements is set forth in the table below:

TABLE 14

| | FORCE at | |
|---|---|---|
| | Max.Load | EXTENSION |
| | (Newtons)/ | at Max.Load |
| Sample | (Pounds) | (mm/Inch) |
| Arrowhead Gusset | 18.3/4.1 | 5.1/0.2 |
| Design | | |
| Arrowhead Web | 47.4/10.7 | 9.5/0.4 |
| Design | | |

It has been demonstrated that by changing the geometry of the fixation element or retention member, the pullout force from the hole or defect in the annular wall can be engineered or changed and any design requirements to success can be met. The test provided an understanding of the load that the fixation element can withstand before being pulled out from the annulotomy hole such as that in a sheep.

In the second part of this experiment, fixation elements or retention members or fixation members shaped as FIG. 21 (Arrowhead Web design) were placed using methods similar to those described in Example 8 and into defects created in the annulus of sheep cadaver lumbar spine After placement, the implants were subjected to axial compression and c-lateral bending. The discs were pressurized by injecting saline in the disc cavity during testing. The pressurization started at 50 psi for the first 100 cycles and ramped or increased by 50 psi at each additional 100 cycles. The maximum pressure reached was about 250 psi. Two loading conditions of (1) 500 cycles/500N and (2) 500 cycles/1000N were tested to investigate whether the fixation elements or retention members were able to withstand these loading conditions. At a load of 500 cycles/500 N, and for 500 cycles/1000 N no extrusion was observed for fixation elements or retention members for the all the pressures that were tested.

This experiments show that fixation elements or retention members can withstand both static and dynamic loading to high degree of loading.

While illustrative embodiments of the invention have been described above, it is, of course, understood that many and various modifications will be apparent to those in the relevant art, or may become apparent as the art develops. Such modifications are contemplated as being within the spirit and scope of the invention or inventions disclosed in this specification.

We claim:

1. An implant for spinal annular repair, which comprises:
a base member and a retention component coupled to the base member and adapted for implantation and fixation into spinal annular tissue,
wherein the retention component is resistive to expulsion from the spinal annular tissue.

2. The implant of claim 1, wherein the base member has an implantation configuration and a fixation configuration.

3. The implant of claim 1, wherein the base member comprises an at least partially cylindrical member comprised of a biodurable, resilient, elastomeric reticulated matrix.

4. The implant of claim 3, wherein, when the elastomeric matrix is compressed from a relaxed configuration to a first, compact configuration for delivery via a delivery device, it expands to a second, working configuration, in vitro, of at least about 30% of the size of the relaxed configuration in at least one dimension.

5. The implant of claim 3, wherein the elastomeric matrix is hydrophobic.

6. The implant of claim 3, wherein the elastomeric matrix is selected from the group consisting of polycarbonate polyurethane-urea, polycarbonate polyurea-urethane, polycarbonate polyurethane, or polycarbonate-polysiloxane polyurethanes, polycarbonate-polysiloxane polyurethane-ureas, polysiloxane polyurethanes, polysiloxane polyurethane-ureas, polycarbonate-hydrocarbon polyurethanes, polycarbonate-hydrocarbon polyurethane-ureas, and mixtures of two or more thereof.

7. The implant of claim 6, wherein the elastomeric matrix comprises a polycarbonate polyurethane or polycarbonate polyurethane-urea.

8. The implant of claim 7, wherein the elastomeric matrix comprises polyurethane.

9. The implant of claim 3, wherein the elastomeric composition comprises a reticulated elastomeric matrix comprising a plurality of pores, the pores having an average diameter or other largest transverse dimension of at least about 20 μm.

10. The implant of claim 9, wherein the pores have an average diameter or other largest transverse dimension of from about 20 μm to about 150 μm.

11. The implant of claim 9, wherein the pores have an average diameter or other largest transverse dimension of from about 150 μm to about 250 μm.

12. The implant of claim 9, wherein the pores have an average diameter or other largest transverse dimension of from about 250 μm to about 500 μm.

13. The implant of claim 3, wherein the elastomeric matrix has a density of from about 2.0 to about 8.5 lbs/ft³.

14. The implant of claim 3, wherein the elastomeric matrix has a compressive strength at 50% compression of from about 1 to about 100 psi.

15. The implant of claim 3, wherein the elastomeric matrix has a tensile strength of from about 1 to about 200 psi and an ultimate tensile elongation in the range of from about 70 to about 300%.

16. The implant of claim 3, wherein the elastomeric composition has a compression set after 22 hours compression at about 25° C. to 50% of its thickness in one dimension of not more than about 30%.

17. The implant of claim 3, wherein the reticulated elastomeric matrix is configured to permit cellular and/or tissue ingrowth and proliferation into the elastomeric matrix.

18. The implant of claim 3, wherein the reticulated elastomeric matrix is endoporously coated with a coating material selected to encourage cellular and/or tissue ingrowth and proliferation.

19. The implant of claim 18, wherein the coating material comprises a coating of a biodegradable material, the biodegradable material comprising collagen, fibronectin, elastin, hyaluronic acid, or mixtures thereof.

20. The implant of claim 18, wherein the coating material comprises a coating of a non-biodegradable material.

21. The implant of claim 3, which comprises two or more reticulated elastomeric matrices having different properties.

22. The implant of claim 1, wherein the base member comprises a material selected from the group consisting of a biodurable material, a biodurable reticulated resilient elastomeric material, a resiliently compressible material, an elastomeric material, a reticulated material, and a material comprising two or more of the properties of the foregoing materials.

23. The implant of claim 1, wherein the retention component is integral to the base member.

24. The implant of claim 23, wherein the retention component comprises a biodurable reticulated elastomeric material.

25. The implant of claim 1, wherein the retention component comprises a separate fixation member.

26. The implant of claim 25, wherein the retention component comprises is a separate fixation member and can comprise a metal, alloy, polymer or other physiological acceptable material.

27. The implant of claim 26, wherein the fixation member comprises a biodegradable polymer.

28. The implant of claim 25, wherein the fixation member has an implantation configuration and a fixation configuration

29. The implant of claim 28, wherein the implantation configuration and the fixation configuration of the fixation member comprise respective configurations that are substantially similar.

30. The implant of claim 25, wherein the fixation member also had a relaxed configuration and the relaxed configuration, implantation configuration, and the fixation configuration of the fixation member comprise respective configurations that are substantially similar.

31. The implant of claim 25, wherein the fixation member comprises at least one fixation element for resistance to expulsion.

32. The implant of claim 31, wherein the at least one fixation element comprises at least two fixation members.

33. The implant of claim 31, wherein the base member comprises a distal portion and the at least one fixation element is disposed at least in part at the distal portion of the base member.

34. The implant of claim 25, wherein the fixation member is disposed substantially along a major dimension of the base member.

35. The implant of claim 28, wherein the resistance to expulsion in the fixation configuration is greater than the resistance to expulsion in the spinal annular tissue when in the implantation configuration.

36. The implant of claim 25, wherein the fixation member is at least partially contained in the base member.

37. The implant of claim 1, wherein the spinal annular tissue comprises a defect or aperature and the deployment of the implant is into the defect or aperature, and wherein, when the implant is in the defect or aperature, the base member of the implant substantially seals or obliterates the defect or aperature, to repair, reconstruct, or reinforce the defect or aperature.

38. The implant of claim 1, wherein at least a portion of the retention component has a resilient compressibility that allows the implantable device to be compressed from a first relaxed configuration to a second configuration during implantation and to expand to a third working configuration when in the fixation position.

39. The implant of claim 38, in which at least one portion of the retention component recovers from the second configuration to at least 30% of the size of the relaxed configuration.

40. The implant of claim 1, wherein at least the retention component has a substantially similar in shape and size during delivery and implantation and in the fixation position.

41. The implant of claim 1, wherein the retention component is at least partially contained in a base member.

42. The implant of claim 1, wherein the implant is adapted for use in repair and/or reconstruction and/or reinforcement of a spinal annulo-nuclear defect or aperature.

43. The implant of claim 42, wherein the annulo-nuclear defect comprises an interface between the nucleus and the defect or aperature, and the retention component has at least a portion for seating at the interface between the nucleus and the defect or aperature.

44. The implant of claim 42, wherein the annulo-nuclear defect or aperature comprises the nuclear space, and the retention component has at least a portion for seating at the nucleus.

45. The implant of claim 42, wherein the annulo-nuclear defect or aperature comprises the annulus, and the retention component has at least a portion for seating at the annulus.

46. The implant of claim 1, wherein the base member comprises reticulated matrices for encouraging cellular or tissue ingrowth.

47. The implant of claim 1, wherein the implant is adapted to mechanically stabilize and strengthen the annular portion of the spinal annular tissue defect.

48. The implant of claim 1, wherein the implant conforms to a surgical and/or pathologic present fissure, aperature, or tear of the spinal annular tissue.

49. The implant of claim 1, wherein the implant stabilizes the nuclear portion of the spinal annular tissue after discectomy.

50. The implant of claim 1, wherein the retention component has a bias structure, in which a first energy is stored when in the implantation configuration, and has a resistance to expulsion when in the fixation configuration; and the retention component has a second stored energy component when in the fixation configuration.

51. The implant of claim 1, wherein the retention component comprises a fixation member having a proximal end and a distal end, and the fixation member comprises at least one projection located in the vicinity of the distal end.

52. The implant of claim 51 wherein the at least one projection has a respective major axis having a directional component that is oriented towards the proximal end.

53. The implant of claim 1, wherein the retention component comprises a fixation member having one or more fixation elements that project into the spinal annular tissue when the retention member is in the fixation configuration.

54. The implant of claim 53 wherein the fixation elements are at least partially compressed when the fixation member is in the implantation position and the compression is at least partially released when the fixation member is in the fixation position.

55. The implant of claim 53 wherein the fixation elements are at least partially collapsed when the fixation member is in the implantation configuration and at least partially expanded when the fixation member is in the fixation configuration.

56. The implant of claim 53 wherein the one or more fixation elements do not project beyond the surface of the body when in the implantation position.

57. The implant of claim 1, wherein the retention component comprises a longitudinal member.

58. The implant of claim 1 which can be rotated in one direction to engage tissue and in another direction to disengage tissue.

59. A system for treating a spinal annular defect which comprises an implant of claim 1 and a delivery means.

60. The system of claim 59, wherein the delivery means is a cannula, trocar, catheter, laproscope, or endoscope.

61. A method for securing a medical apparatus, the apparatus comprising a retention member adapted for deployment into a spinal annular tissue defect, the retention member having a implanation position and a fixation position, and being resistive to expulsion in the fixation position, the method comprising the steps of
(a) positioning the apparatus with respect to the spinal annular tissue defect with a delivery device;
(b) deploying the apparatus; and
(c) at least partially fixating the retention member.

62. An implant for spinal annular repair, comprising a member comprising resilient elastomeric material adapted for retaining the implant in an annular defect or aperature, the annular defect or aperature having an annular defect or aperature wall, wherein the member has an implantation position and a fixation position, the member being adapted to being in a first state prior to being placed in the implantation position, and a second state when in the fixation position, and wherein the member forms a seal with the annular wall when in the second state.

63. The implant of claim 62, wherein the first state comprises a state of compression in at least one dimension, and the second state comprises a state of at least partial reexpansion.

64. The implant of claim 62, wherein the seal comprises a conformal or frictional seal.

65. The implant of claim 62, wherein the member of resilient elastomeric material comprises a reticulated material.

66. The implant of claim 65, wherein the member of resilient elastomeric material comprises a biodurable material.

67. The implant of claim 65, wherein the annular defect further comprises an annulo-nuclear opening, and wherein the member protrudes through the annular defect or aperature beyond the annulo-nuclear opening when in the fixation position, and the implant further comprising a portion coupled to the member adapted to protrude beyond the annulo-nuclear opening when the member is in the fixation position, the portion protruding beyond the annulo-nuclear opening being expanded further than the member when in the second reexpanded position.

68. The apparatus of claim 62, wherein the portion protruding beyond the annular nuclear opening has a cross-sectional shape that differs from the cross sectional shape of the member.

69. The apparatus of claim 62, further comprising a retention member coupled to the member and adapted for implantation and fixation into a spinal annular tissue.

70. The apparatus of claim 69, wherein the fixation is into the annular defect wall.

71. The apparatus of claim 62, further comprising a retention member coupled to the member and adapted for implantation and fixation into a spinal annular tissue.

72. The apparatus of claim 71, wherein the fixation is into an annular tissue at the annular nuclear opening.
